(19) United States
(12) Patent Application Publication
(10) Pub. No.: US 2017/0071930 A1
(43) Pub. Date: Mar. 16, 2017

(54) PHARMACEUTICAL COMPOSITIONS OF CETP INHIBITORS

(71) Applicant: DR. REDDY'S LABORATORIES LTD., Andhra Pradesh (IN)

(72) Inventors: Bijay Kumar Padhi, Orissa (IN); Rajesh Gandhi, Madhya Pradesh (IN); Swati Arun Guhagarkar, Maharashtra (IN); Rajeev Singh Raghuvanshi, Haryana (IN)

(21) Appl. No.: 14/443,525

(22) PCT Filed: Nov. 19, 2013
(86) PCT No.: PCT/IB2013/002909

§ 371 Date: May 18, 2015

(30) Foreign Application Priority Data
Nov. 19, 2012 (IN) 4811/CHE/2012

(51) Int. Cl. A61K 31/4709; A61K 9/20; A61K 9/48

(52) U.S. Cl. CPC A61K 31/4709; A61K 9/4808; A61K 9/4866; A61K 9/485; A61K 9/4858; A61K 9/2077; A61K 9/2009; A61K 9/2013; A61K 9/2018; A61K 9/2054

(57) ABSTRACT

The present application discloses a pharmaceutical composition comprising a class of CETP inhibitors with improved oral bioavailability. The application further discloses compositions comprising a class of CETP inhibitor and at least one solubility improving material and optionally one or more wetting agents.

### FIELD OF THE APPLICATION

This invention is directed to pharmaceutical compositions containing cholesteryl ester transfer protein (CETP) inhibitors. This invention is further directed to the use of such compositions to elevate certain plasma lipid levels, including high density lipoprotein (HDL)-cholesterol and to lower certain other plasma lipid levels, such as low density lipoprotein (LDL)-cholesterol and triglycerides. Thus, this invention is also directed to treat diseases which are affected by low levels of HDL cholesterol and/or high levels of LDL-cholesterol and triglycerides, such as atherosclerosis and cardiovascular diseases.

### BACKGROUND

Hyperlipidemia or an elevation in serum lipids is associated with an increase incidence of cardiovascular disease and atherosclerosis. Primary hyperlipidemia is a term used to describe a defect in lipoprotein metabolism. The lipoproteins commonly affected are low density lipoprotein (LDL) cholesterol, which transports mainly cholesterol, and very low density lipoprotein-cholesterol (VLDL-cholesterol), which transports mainly triglycerides (TG). Most subjects with hyperlipidemia have a defect in LDL metabolism, characterized by raised cholesterol, LDL-C levels, with or without raised triglyceride levels; such subjects are termed hypercholesterolemic (Fredrickson Type II). Familial hypercholesterolemia (FH) is caused by any one of a number of genetically-determined defects in the LDL receptor, which is important for the entry of cholesterol into cells. The condition is characterized by a reduced number of functional LDL receptors, and is therefore associated with raised serum LDL-C levels due to an increase in LDL.

It is reasonably known in the art that the likelihood of cardiovascular disease can be decreased, if the serum lipids, and in particular LDL-C, can be reduced. It is further known that the progression of atherosclerosis can be retarded or the regression of atherosclerosis can be induced if serum lipids can be lowered. In such cases, individuals diagnosed with hyperlipidemia or hypercholesteremia should consider lipid-lowering therapy to retard the progression or induce the regression of atherosclerosis for purposes of reducing their risk of cardiovascular disease, and in particular coronary artery disease.

Cholesteryl ester-transfer protein (CETP) is an important player in metabolism of lipoproteins, such as, for example, a high density lipoprotein (HDL). CETP is a 70 kDa plasma glycoprotein that is physically associated with HDL particles. It facilitates the transport of cholesteryl ester from HDL to apolipoprotein B-containing lipoproteins. This transfer is accompanied by transfer of triglycerides in the opposite direction. Thus, a decrease in CETP activity can result in an increase in the level of HDL cholesterol and a decrease in the level of very low density lipoprotein (VLDL) and low density lipoprotein (LDL). CETP can therefore simultaneously affect the concentrations of pro-atherogenic (for example, LDL) and anti-atherogenic (for example, HDL) lipoproteins.

Several CETP inhibitors are currently in various clinical phases of development for treating various aforementioned disorders. In spite of having various advantages, CETP inhibitors are proven to be difficult to formulate for oral administration. CETP inhibitors are of a highly lipophilic nature and have extremely low solubility in water. Due to their poor solubility, bioavailability of conventional oral compositions is very poor. The lipophilic nature of CETP inhibitors not only leads to low solubility but also tends to poor wettability, further reducing their tendency to be absorbed from the gastrointestinal tract. In addition to the low solubility, CETP inhibitors also tend to have significant, “food effect”, where a significant difference in rate and amount of drug absorption is observed when the drug is administered with or without a meal. This “food effect”, often complicates the dosing regimen and may require high dosing to achieve the desired therapeutic effect, resulting in potentially unwanted side effects.

Several attempts have been made to improve the solubility of CETP inhibitors, but have generally ended up with limited success. At the outset, most methods aimed at enhancing aqueous concentration and bioavailability of low-solubility drugs only offer moderate improvements. References describing improving the dissolution of poorly soluble drugs include: U.S. Pat. Nos. 5,474,989, 5,456,923, 5,985,326, 6,638,522, 6,730,679, 6,350,786, 6,548,555, 7,037,528, 7,078,057, 7,034,013, 7,008,640, 7,081,255, and 8,030,359.

In view of the foregoing, there remains a long felt need for developing compositions containing CETP inhibitors with improved bioavailability and minimal or less food effect.

### SUMMARY

In one aspect, the present application relates to a pharmaceutical composition comprising:

a) a CETP inhibitor having formula (I) or (Ia′) or (II) or (III),

b) at least one solubility improving material,

c) optionally one or more wetting agents, and

d) at least one pharmaceutically acceptable excipient.

In another aspect, the present application provides a composition in which the CETP inhibitor of formula (I), (Ia′), (II) or (III) is combined with at least one solubility improving material in a sufficient amount so that the composition provides maximum drug availability for absorption.

In yet another aspect, the present application provides a composition comprising solid amorphous dispersion of CETP inhibitor of formula (I), (Ia′), (II) or (III) and at least one solubility improving material.

In one embodiment, the present application provides a composition comprising a CETP inhibitor of formula (I), (Ia′), (II) or (III) and at least one solubility improving material, wherein said composition
releases not more than 50% at a period of 30 minutes or
releases not more than 75% at a period of 60 minutes or
releases not less than 90% at a period of 360 minutes in 900 ml of simplified simulated intestinal fluid having a pH of 6.5, when tested in a USP Type 2 apparatus at 25 rpm and 37° C.

In another embodiment, the present application provides a composition comprising a CETP inhibitor of formula (I), (Ia′), (II) or (III) and at least one solubility improving material, wherein said composition when administered to a mammal provides the area under the curve (AUC₀₋₄₈) profile in fed to fast state in a ratio of about 1 to 3.

In yet another embodiment, the present application provides a composition comprising a CETP inhibitor of formula (I), (Ia′), (II) or (III) and at least one solubility improving material, wherein said composition when administered to a mammal provides the maximum plasma profile (Cₘₐₓ) in fed to fast state in a ratio of about 1 to 3.

In another aspect, the present application provides a method of administering to a patient a pharmaceutical composition as described herein.

### DESCRIPTION OF DRAWINGS

FIG. 1 shows the comparative XRD data of Example 6, placebo and drug.

FIG. 2 shows the comparative XRD data of Example 12, placebo and drug.

FIG. 3 shows the comparative pharmacokinetic data of Example 6, in fed and fasted state.

FIG. 4 shows the comparative pharmacokinetic data of Example 11, in fed and fasted state.

FIG. 5 shows the comparative pharmacokinetic data of Example 12, in fed and fasted state.

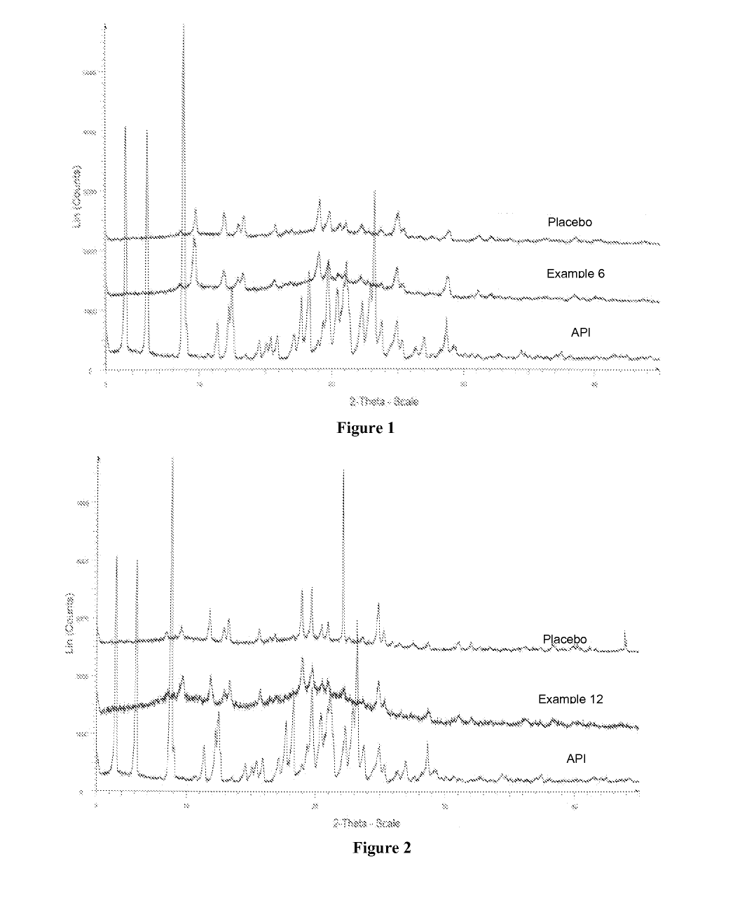

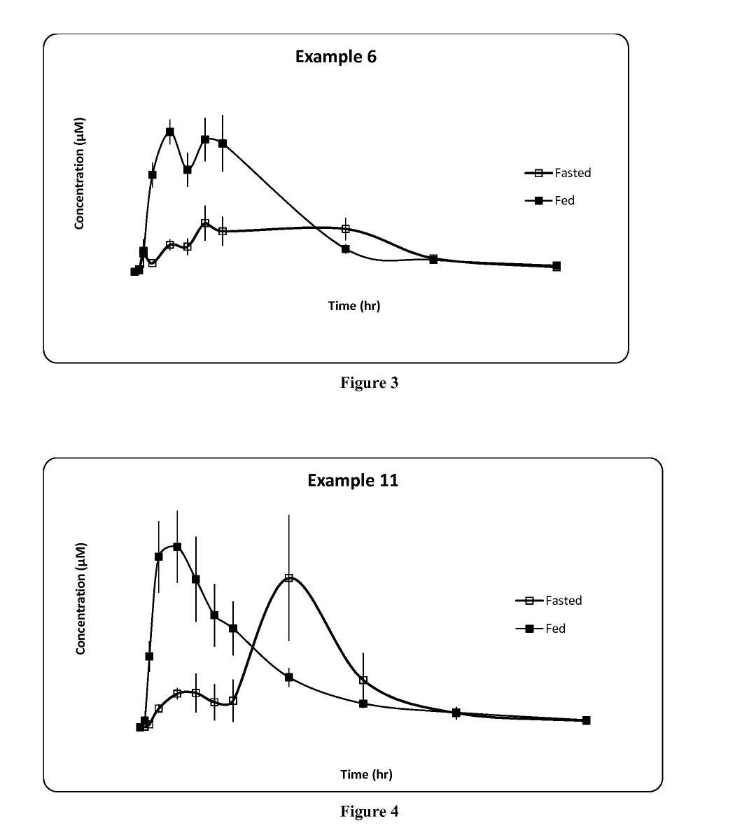

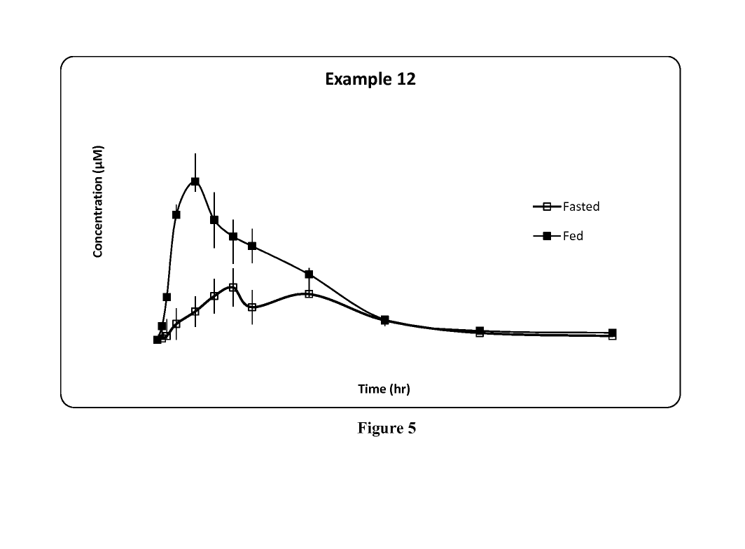

### DETAILED DESCRIPTION

The present application will be described in more detail below.

While the specification concludes with the claims particularly pointing and distinctly claiming the invention, it is believed that the present invention will be better understood from the following description. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having,” “including,” and “comprised of” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Generally, such additives may not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute. Such terms will be defined by the circumstances and the terms that they modify as those terms are understood by those of skill in the art. This includes, at very least, the degree of expected experimental error, technique error and instrument error for a given technique used to measure a value.

The definitions of the groups and other variables mentioned in formula (I) and (Ia′) are as defined in US2006/0178514 and are described in detail below.

Definitions of the groups and other variables mentioned in formula (I) have the meaning as defined below:

The terms “halogen” or “halo” includes fluorine, chlorine, bromine, or iodine.

The term “alkyl” group is used to refer to both linear and branched alkyl groups. Exemplary alkyl groups include, but are not limited to, methyl, ethyl, propyl, butyl, pentyl, hexyl, heptyl, octyl, nonyl, or decyl, and the like. Unless otherwise specified, an alkyl group has from 1 to 12 carbon atoms. Also unless otherwise specified, all structural isomers of a given structure, for example, all enantiomers and all diasteriomers, are included within this definition. For example, unless otherwise specified, the term propyl is meant to include n-propyl and iso-propyl, while the term butyl is meant to include n-butyl, iso-butyl, t-butyl, sec-butyl, and so forth.

The term “aryl” refers to an optionally substituted monocylic or polycyclic aromatic ring system of 6 to 14 carbon atoms. Exemplary groups include phenyl, naphthyl, 1,2,3,4-tetrahydronaphthalene, indane, fluorene, and the like. Unless otherwise specified, an aryl group typically has from 6 to 14 carbon atoms.

“Aralkyl” refers to an aryl substituted alkyl group, wherein the aryl group and the alkyl group are defined herein. Typically, the aryl group can have from 6 to 14 carbon atoms, and the alkyl group can have up to 10 carbon atoms. Exemplary aralkyl groups include, but are not limited to, benzyl, phenylethyl, phenylpropyl, phenylbutyl, propyl-2-phenylethyl and the like.

The term “haloalkyl” refers to a group containing at least one halogen and an alkyl portion as define above, that is, a haloalkyl is a substituted alkyl group that is substituted with one or more halogens. Unless otherwise specified, all structural isomers of a given structure, for example, all enantiomers and all diasteriomers, are included within this definition. Exemplary haloalkyl groups include fluoromethyl, chloromethyl, fluoroethyl, chloroethyl, trifluoromethyl, and the like. Unless otherwise specified, a haloalkyl group has from 1 to 12 carbon atoms.

A “cycloalkyl” group refers to a cyclic alkyl group which can be mono or polycyclic. Exemplary cycloalkyl groups include cyclopropyl, cyclobutyl, cyclopentyl, cyclohexyl, cycloheptyl, cyclooctyl, cyclononyl, and cyclodecyl. Unless otherwise specified, a cycloalkyl group has from 3 to 12 carbon atoms.

An “alkoxy” group refers to an —O(alkyl) group, where alkyl is as defined herein. Therefore, unless otherwise specified, all isomers of a given structure are included within a definition. Exemplary alkyl groups include methoxy, ethoxy, n-propoxy, iso-propoxy, n-butoxy, iso-butoxy, t-butoxy, and the like. Unless otherwise specified, an alkoxy group has from 1 to 12 carbon atoms. Unless otherwise specified, all structural isomers of a given structure, for example, all enantiomers and all diasteriomers, are included within this definition. For example, unless otherwise specified, the term propoxy is meant to include n-propoxy and iso-propoxy.

An “aryloxy” group refers to an —O(aryl) group, where aryl is as defined herein. Thus, the aryl portion of an aryloxy group can be substituted or unsubstituted. Exemplary aryloxy groups include, but are not limited to, phenoxy, naphthyl, and the like. Unless otherwise specified, an aryloxy group typically has from 6 to 14 carbon atoms.

“Haloalkoxy” refers to an alkoxy group with a halo substituent, where alkoxy and halo groups are as defined above. Exemplary haloalkoxy groups include fluoromethoxy, chloromethoxy, trifluoromethoxy, trichloroethoxy, fluoroethoxy, chloroethoxy, trifloroethoxy, perfluoroethoxy (—OCF₂CF₃), trifluoro-t-butoxy, hexafluoro-t-butoxy, perfluoro-t-butoxy (—OC(CF₃)₃), and the like. Unless otherwise specified, an haloalkoxy group typically has from 1 to 12 carbon atoms.

“Alkylthio” refers to an —S(alkyl) group, where alkyl group is as defined above. Exemplary alkyl groups include methylthio, ethylthio, propylthio, butylthio, iso-propylthio, iso-butylthio, and the like. Unless otherwise specified, an alkylthio group typically has from 1 to 12 carbon atoms.

“Heteroaryl” is an aromatic monocyclic or polycyclic ring system of 4 to 10 carbon atoms, having at least one heteroatom or heterogroup selected from —O—, >N—, —S—, >NH or NR, and the like, wherein R is a substituted or unsubstituted alkyl, aryl, or acyl, as defined herein. In this aspect, >NH or NR are considered to be included when the heteroatom or heterogroup can be >N—. Exemplary heteroaryl groups include as pyrazinyl, isothiazolyl, oxazolyl, pyrazolyl, pyrrolyl, triazolyl, tetrazolyl, oxatriazolyl, oxadiazolyl, pyridazinyl, thienopyrimidyl, furanyl, indolyl, isoindolyl, benzo[1,3]dioxolyl, 1,3-benzoxathiole, quinazolinyl, isoquinolinyl, quinolinyl, pyridyl, 1,2,3,4-tetrahydro-isoquinolinyl, 1,2,3,4-tetrahydro-quinolinyl pyridyl, thiophenyl, and the like. Unless otherwise specified, a heteroaryl group typically has from 4 to 10 carbon atoms. Moreover, the heteroaryl group can be bonded to the heterocyclic core structure at a ring carbon atom, or, if applicable for a N-substituted heteroaryl such as pyrrole, can be bonded to the heterocyclic core structure through the heteroatom that is formally deprotonated to form a direct heteroatom-pyrimidine ring bond.

“Heterocyclyl” is a non-aromatic, saturated or unsaturated, monocyclic or polycyclic ring system of 3 to 10 member having at least one heteroatom or heterogroup selected from —O—, >N—, —S—, >NR, >SO₂, >CO₃ and the like, wherein R is hydrogen or a substituted or an unsubstituted alkyl, aryl, or acyl, as defined herein. Exemplary heterocyclyl groups include aziridinyl, imidazolidinyl, 2,5-dihydro-[1,2,4]oxadiazolenyl, oxazolidinyl, isooxazolidinyl, pyrrolidinyl, piperdinyl, piperazinyl, morpholinyl, thiomorpholinyl, thiazolidinyl, 1,3-dioxolanyl, 1,4-dioxanyl, 2,5-dihydro-1H-imidazolyl, and the like. Unless otherwise specified, a heterocyclyl group typically has from 2 to 10 carbon atoms. A heterocyclyl group can be bonded through a heteroatom that is formally deprotonated or a heterocyclyl group can be bonded through a carbon atom of the heterocyclyl group.

“Heterocycloalkyl” refers to the saturated subset of a heterocyclyl, that is, a non-aromatic, saturated monocyclic or polycyclic ring system of 3 to 10 members having at least one heteroatom or heterogroup selected from —O—, >N—, —S—, >NR, >SO₂, >CO₃ and the like, wherein R is hydrogen or a substituted or an unsubstituted alkyl, aryl, or acyl, as defined herein. Exemplary heterocycloalkyl groups include aziridinyl, piperdinyl, piperazinyl, morpholinyl, thiazolidinyl, 1,3-dioxolanyl, 1,4-dioxanyl, and the like. Unless otherwise specified, a heterocycloalkyl group typically has from 2 to 10 carbon atoms, or in another aspect, from 2 to 6 carbon atoms. A heterocycloalkyl group can be bonded through a heteroatom that is formally deprotonated or a heterocycloalkyl group can be bonded through a carbon atom of the heterocycloalkyl group.

A “heteroaryloxy” group refers to an aryloxy-type analog of a heteroaryl group. Thus, a heteroaryloxy group is intended to describe a heteroaryl group as defined herein, that is bonded to an oxygen atom, to form a formal [O-heteroaryl] moiety. Unless otherwise specified, a heteroaryloxy group typically comprises from 4 to 10 carbon atoms.

A “cyclic” moiety, including a monocyclic moiety or a bicyclic moiety, unless otherwise specified, is intended to be inclusive of all the cyclic groups disclosed herein, for example, a heteroaryl group, a heterocyclyl group, a heterocycloalkyl group, and/or a heteroaryloxy group.

An “alkoxycarbonyl” group refers to a —C(O)O(alkyl) group, wherein the alkyl portion of the alkoxycarbonyl group is defined as herein. Examples of alkoxycarbonyl groups include, but are not limited to, methoxycarbonyl, ethoxycarbonyl, t-butoxycarbonyl and the like.

An “alkenyl” group is an aliphatic hydrocarbon group comprising an alkene functionality, regardless of the regiochemistry of the alkene functionality within the aliphatic hydrocarbon group. Unless otherwise specified, an alkenyl group typically has from 2 to 12 carbon atoms, and in another aspect, is a C₂-C₁₀ alkenyl group. Exemplary alkenyl groups include ethenyl, propenyl, butenyl, and the like, including all regiochemistries, thus, “butenyl” includes 1-butenyl, 2-butenyl, and 3-butenyl.

An “alkynyl” group is an aliphatic hydrocarbon group comprising an alkyne functionality, regardless of the regiochemistry of the alkyne functionality within the aliphatic hydrocarbon group. Unless otherwise specified, an alkynyl group typically has from 2 to 12 carbon atoms, and in another aspect, is a C₂-C₁₀ alkynyl group. Exemplary alkynyl groups include ethynyl, propynyl, butynyl, and the like, including all regiochemistries. Thus, “butynyl” includes 1-butynyl, 2-butynyl, and 3-butynyl.

An “alkoxyalkyl” group is an alkoxy-substituted alkyl group, wherein an alkoxy group and an alkyl group are defined herein. Unless otherwise specified, an alkoxyalkyl group typically has from 2 to 20 carbon atoms. In one aspect, an alkoxyalkyl group can be a (C₁-C₁₀) alkoxy group bonded to a (C₁-C₁₀) alkyl group, where alkoxy and alkyl groups are as defined here, including all stereochemistries and all regiochemistries. Exemplary alkoxyalkyl groups include methoxymethyl, methoxyethyl, methoxypropyl, ethoxymethyl, ethoxyethyl, methoxyisopropyl, ethoxyisobutyl, and the like.

An “aminoalkyl” group, as used herein, refers to an amino-substituted alkyl group, wherein an alkyl is defined herein. Unless otherwise specified, an aminoalkyl group can typically have from 1 to 12 carbon atoms, therefore, a typical aminoalkyl group can be an amino (C₁-C₁₂) alkyl, including all regiochemistries. Exemplary aminoalkyl groups include, but are not limited to, aminomethyl, aminoethyl, aminopropyl, and the like.

A “cycloalkyl-substituted alkyl” group, also termed a “cycloalkylalkyl” group, refers to an alkyl group that is substituted with a cycloalkyl substituent, wherein alkyl and cycloalkyl are defined herein. Thus, the cycloalkyl group portion can be a mono or polycyclic alkyl group. Unless otherwise specified, a cycloalkylalkyl group can have up to 20 carbon atoms, regardless of how the carbon atoms are distributed between the alkyl portion and the cycloalkyl portion of the group, and including all possible stereochemistries and all regiochemistries. For example, in one aspect, a cycloalkyl-substituted alkyl can comprise a (C₃-C₁₀) cycloalkyl bonded to a C₁-C₁₀ alkyl group, wherein the cycloalkyl portion can be mono or polycyclic. Exemplary cycloalkylalkyl groups include, but are not limited to, cyclopropylmethyl, cyclopropylethyl, cyclopropylpropyl, cyclobutylmethyl, cyclobutylethyl, cyclobutylpropyl, cyclopentylmethyl, cyclopentylethyl, cyclopentylpropyl, cyclohexylmethyl, cyclohexylethyl, cyclohexylpropyl, cycloheptylmethyl, cycloheptylethyl, cyclooctylmethyl, cyclooctylethyl, cyclooctylpropyl, and the like.

A “cycloalkoxy” group, also referred to as a “cycloalkyloxy” group, refers herein to an —O(cycloalkyl) substituent, that is, an alkoxide-type moiety comprising a cycloalkyl group, wherein a cycloalkyl is defined herein. Thus, the cycloalkyl group portion can be a mono or polycyclic alkyl group, and unless otherwise specified, a cycloalkylalkyl group can have up to 20 carbon atoms. In one aspect, a cycloalkoxy group can be a (C₃-C₁₀) cycloalkyl-O— group. Exemplary cycloalkoxy groups include cyclopropoxy, cyclobutoxy, cyclopentoxy, cyclohexoxy, and the like.

An “acyl” group refers to a (C₁-C₁₀) alkyl-CO— group, wherein the (C₁-C₁₀) alkyl group is used in this structure to refer to the alkyl-linker moiety bonded both to the CO group, and to another chemical group. Examples of acyl groups include, but are not limited to, methylcarbonyl, ethylcarbonyl, propylcarbonyl, isopropylcarbonyl, and the like.

An “alkenylene” group refers to a (C₂-C₁₀) hydrocarbon linker comprising at least one C═C double bond within the C₂-C₁₀ chain. Examples of alkenylene groups include, but are not limited to, —CH═CH—, —CH₂—CH═CH, —CH₂—CH═CH—CH₂—, —CH₂—CH═CH—CH═CH—, and the like. Thus, unless otherwise specified, an alkenylene group has from 2 to 10 carbon atoms.

A “haloalkoxyalkyl” group refers to a haloalkyl-O—(C₁-C₁₀)alkyl group, that is, a haloalkoxy-substituted alkyl group, wherein haloalkoxy and alkyl are defined herein. Unless otherwise specified, a cycloalkylalkyl group can have up to 20 carbon atoms, regardless of how the carbon atoms are distributed between the haloalkoxy portion and the alkyl portion of the group, and including all possible stereochemistries and all regiochemistries. In one aspect, for example, a haloalkoxyalkyl is haloalkyl-O—(C₁-C₁₀)alkyl, where group can be (C₁-C₁₀) haloalkyl group bonded to a (C₁-C₁₀) alkyl moiety. Exemplary haloalkoxyalkyl groups include trifluoromethoxymethyl, chloromethoxyethyl, flouroethoxyethyl, chloroethoxyethyl, trilfluoromethoxypropyl, hexafluoroethoxyethyl and the like.

A “monoalkylamino” group refers to an amino group that is substituted with a single alkyl group, that is, a mono(C₁-C₂₀)alkylamino group. Unless otherwise specified, a monoalkylamino group can have up to 20 carbon atoms. In one aspect, a monoalkylamino group can be a (C₁-C₁₀)alkyl-substituted amino group. Exemplary monoalkylamino groups include methylamino, ethylamino, propylamino, isopropylamino, and the like.

A “dialkylamino” group refers to an amino group that is substituted with two, independently-selected, alkyl groups, that is, a di (C₁-C₁₀) alkylamino group. Unless otherwise specified, a dialkylamino group can have up to 20 carbon atoms. Exemplary dialkylamino groups include dimethylamino, diethylamino, and the like.

Definitions of the groups and other variables mentioned in formula (II) and (III) have the meaning as defined below:

As used herein, the expression ‘alkyl’ group refers to linear or branched alkyl group with 1 to 10 carbon atoms. Exemplary alkyl group includes, but is not limited to, methyl, ethyl, n-propyl, iso-propyl, n-butyl, iso-butyl, t-butyl, n-pentyl, iso-pentyl, hexyl, heptyl, octyl and the like.

As used herein, the expression ‘alkoxy’ group refers to an —O (alkyl) group, wherein alkyl group is as defined above. Exemplary alkoxy groups include methoxy, ethoxy, n-propoxy, iso-propoxy, n-butoxy, iso-butoxy, t-butoxy, and the like. Unless otherwise specified, an alkoxy group has from 1 to 10 carbon atoms.

As used herein, the expression ‘alkoxyalkyl’ means at least one alkoxy group is substituted on an alkyl group. Both alkoxy and alkyl have the meaning as defined above. Representative examples of alkoxyalkyl groups include, but are not limited to, ethoxymethyl, methoxyethyl, isopropoxyethyl, 2-methoxybut-1-yl, 3,3-dimethoxyprop-1-yl, and the like. Unless otherwise specified, an alkoxyalkyl group typically has from 1 to 10 carbon atoms.

As used herein, the expression ‘acyl’ group refers to alkyl-CO— group, wherein alkyl group is as defined above. Acyl group refers to an alkyl-linker moiety bonded both to the CO group, and to another chemical group. Examples of acyl groups include, but are not limited to, acetyl, propionyl and the like. Acyl group includes formyl group too.

As used herein, the expression ‘aryl’ means substituted or unsubstituted phenyl or naphthyl. Specific examples of substituted phenyl or naphthyl include o-, p-, m-tolyl, 1,2-, 1,3-, 1,4-xylyl, 1-methylnaphthyl, 2-methylnaphthyl, etc. “Substituted phenyl” or “substituted naphthyl” also include any of the possible substituents as further defined herein or one known in the art. Derived expression, “arylsulfonyl,” is to be construed accordingly.

As used herein, the expression ‘Cycloalkyl’ group refers to a cyclic alkyl group which may be mono, bicyclic, polycyclic, or a fused/bridged ring system. Exemplary cycloalkyl groups include, but are not limited to, cyclopropyl, cyclobutyl, cyclopentyl, cyclohexyl, cycloheptyl, cyclooctyl, and the like. Unless otherwise specified, a cycloalkyl group typically has from 3 to about 10 carbon atoms. Typical bridged cycloalkyls include, but are not limited to adamantyl, noradamantyl, bicyclo[1.1.0]butanyl, norbornyl(bicyclo[2.2.1]heptanyl), norbornenyl (bicyclo[2.2.1]heptanyl), norbornadienyl(bicyclo[2.2.1]heptadienyl), bicyclo[2.2.1]heptanyl, bicyclo[3.2.1]octanyl, bicyclo[3.2.1]octadienyl, bicyclo[2.2.2]octanyl, bicyclo[2.2.2]octenyl, bicyclo[2.2.2]octadienyl, bicyclo[5.2.0]nonanyl, bicyclo[4.3.2]undecanyl, tricyclo[5.3.1.1]dodecanyl and the like.

As used herein, the expression ‘halogen or halo’ represents fluorine, chlorine, bromine, or iodine.

As used herein, the expression ‘haloalkyl’ means at least one halogen atom is substituted on an alkyl group. Both halogen and alkyl have the meaning as defined above. Representative examples of haloalkyl groups include, but are not limited to, fluoromethyl, chloromethyl, fluoroethyl, chloroethyl, difluoromethyl, trifluoromethyl, dichloroethyl, trichloroethyl and the like. Unless otherwise specified, a haloalkyl group typically has from 1 to 10 carbon atoms.

As used herein, the expression ‘haloalkoxy’ means at least one halogen atom is substituted on an alkoxy group, wherein alkoxy and halogen groups are as defined above. Exemplary haloalkoxy groups include, but not limited to, fluoromethoxy, chloromethoxy, trifluoromethoxy, trichloroethoxy, fluoroethoxy, chloroethoxy, trifluoroethoxy, perfluoroethoxy (—OCF₂CF₃), trifluoro-t-butoxy, hexafluoro-t-butoxy, perfluoro-t-butoxy (—OC(CF₃)₃), and the like. Unless otherwise specified, a haloalkoxy group typically has from 1 to 10 carbon atoms.

As used herein, the expression ‘heterocycle’ or ‘heterocyclyl’ or ‘heterocyclic’ is a saturated monocyclic or polycyclic ring system of 3 to 10 members having at least one heteroatom or heterogroup selected from —O—, —N—, —S—, —SO₂, or —CO. Exemplary heterocyclyl groups include, but not limited to, azetidinyl, oxazolidinyl, oxazolidinonyl, isoxazolidinyl, imidazolidin-2-onyl, pyrrolidinyl, pyrrolidin-2-onyl, piperidinyl, piperazinyl, morpholinyl, thiomorpholinyl, thiomorpholine-1,1-dioxide, thiazolidinyl, 1,3-dioxolanyl, 1,4-dioxanyl, and the like. Unless otherwise specified, a heterocyclyl group typically has from 3 to about 10 carbon atoms.

As used herein, the expression ‘heteroaryl’ is an unsaturated, aromatic or non-aromatic, monocyclic or polycyclic ring system of 3 to 10 members having at least one heteroatom or heterogroup selected from —O—, —N—, —S—, —SO₂, or —CO. Exemplary heteroaryl groups include, but not limited to, oxazolyl, isoxazolyl, thiazolyl, pyridinyl, pyrrolyl, pyrimidinyl, thiazinyl, pyrazinyl, pyrazolyl, tetrazolyl, imidazothiazolyl, indolizidinyl, indolyl, quinolinyl, quinoxalinyl, benzoxazolyl, benzoisoxazolyl, benzothiazolyl, benzodioxolyl, benzotriazolyl, indazolyl, quinoxalinyl, imidazolyl, pyrazolopyridinyl, and the like. Unless otherwise specified, a heteroaryl group typically has from 3 to about 10 carbon atoms.

As used herein, the expression ‘5-7 membered heterocyclic or heteroaryl group’ represents a heterocyclic or heteroaryl group as defined above having 5-7 ring atoms. Exemplary 5-7 membered heterocyclic or heteroaryl groups include, but not limited to, pyrazolyl, imidazolyl, isoxazolyl, oxazolyl, tetrazolyl, morpholinyl, oxazolidinonyl, and the like.

As used herein, the expression ‘OH’ represents a hydroxy group.

As used herein, the expression ‘CN’ represents a cyano group.

### Cholesterol Ester Transfer Protein (CETP) Inhibitor:

The CETP inhibitors that are essentially aqueous insoluble, highly hydrophobic, and are characterized by a set of physical properties. Several characteristic properties of this class of compounds are
(i) hydrophobic CETP inhibitors have extremely low aqueous solubility. Extremely low aqueous solubility is meant that the minimum aqueous solubility at physiologically relevant pH (pH of 1 to 8) is less than about 10 μg/ml, less than about 2 μg/ml, or less than about 1 μg/ml.
(ii) essentially insoluble, hydrophobic CETP inhibitors are that they are extremely hydrophobic. Extremely hydrophobic is meant that the Clog P value of the drug, has a value of at least 4.0, a value of at least 5.0, or a value of at least 6.0.
(iii) a very high dose-to-solubility ratio. By “very high dose-to-solubility ratio” is meant that the dose-to-solubility ratio has a value of at least 1000 ml, preferably value of at least 5,000 ml, at least 8,000 ml or a value of at least 10,000 ml.
(iv) have very low absolute bioavailability. The absolute bioavailability of drugs in this subclass when dosed orally in their undispersed state is less than about 10% and more often less than about 5%.

Wherever CETP inhibitors are not limited by a particular structural class, the present application is not limited by any particular structure or group of CETP inhibitors. Rather, the application has general applicability to CETP inhibitors as a class, the class tending to be composed of compounds having low solubility.

In one aspect, the present application relates to a pharmaceutical composition comprising:
a) a CETP inhibitor having formula (I) or (Ia′) or (II) or (III),
b) at least one solubility improving material,
c) optionally one or more wetting agents, and
d) at least one pharmaceutically acceptable excipient.

In one embodiment of the above aspect, the present application relates to a pharmaceutical composition comprising:
a) a CETP inhibitor having formula (I),
b) at least one solubility improving material,
c) optionally one or more wetting agents, and
d) at least one pharmaceutically acceptable excipient; wherein formula (I) is defined as follows,

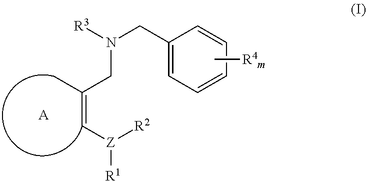

or a stereoisomer thereof or a pharmaceutically acceptable salt thereof, wherein:
A is a substituted or an unsubstituted quinoline moiety having the formula:

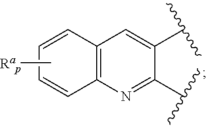

wherein R^{a}, in each occurrence, is selected independently from: 1) a halogen; a hydroxyl, or a cyano; 2) an alkyl or an alkoxy, any of which having up to 12 carbon atoms; or 3) CO₂R⁶; and p is an integer from 0 to 3, inclusive;
R¹ and R² are selected independently from: 1) hydrogen; 2) a substituted or an unsubstituted alkyl, cycloalkyl, haloalkyl, aryl, heterocyclyl, heteroaryl, any of which having up to 12 carbon atoms, wherein any heterocyclyl or heteroaryl comprises at least one heteroatom or heterogroup selected independently from O, N, S, NR¹⁰, SO₂, or CO; 3) CO₂R⁶, COR⁸, SO₂R⁸, SO₂NR⁶R⁷, or CONR⁶R⁷; or 4) (CHR^{x})ₙR⁵ or (CH₂)ₙR^{d}CO₂R^{e}, wherein n, in each occurrence, is 1, 2, or 3; R^{x}, in each occurrence, is selected independently from an alkyl or an alkoxy, either of which having up to 12 carbon atoms, or hydrogen; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, NR¹⁰, SO₂, or CO; and R^{e}, in each occurrence, is selected independently from an alkyl or a cycloalkyl, either of which having up to 12 carbon atoms, or hydrogen;
or R¹ and R² together with the diradical Z to which they are attached_form a substituted or an unsubstituted monocyclic or bicyclic moiety comprising up to 12 carbon atoms, and optionally comprising 1, 2, or 3 heteroatoms or heterogroups in addition to Z, selected independently from O, N, S, NR¹⁰, SO₂, or CO;
R³ is selected from: 1) hydrogen or cyano; 2) a substituted alkyl having up to 12 carbon atoms; 3) a substituted or an unsubstituted aryl, or a substituted or an unsubstituted 5-, 6-, or 7-membered heterocyclyl or heteroaryl, any of which having up to 12 carbon atoms, comprising 1, 2, or 3 heteroatoms or heterogroups selected independently from O, N, S, NR¹⁰, SO₂, or CO; or 4) CO₂R⁶, COR⁸, SO₂R⁸, SO₂NR⁶R⁷, CONR⁶R⁷, C(S)NR⁶R⁷, C(S)NC(O)OR⁸, or C(S)SR⁸; or 5) a substituted or an unsubstituted group selected from 4,5-dihydro-oxazolyl, tetrazolyl, isoxazolyl, pyridyl, pyrimidinyl, oxadiazolyl, thiazolyl, or oxazolyl; wherein any optional substituent is selected independently from: a) an alkyl or haloalkyl, any of which having up to 12 carbon atoms; or b) CO₂R⁹, wherein R⁹ is an alkyl having up to 12 carbon atoms;
wherein when R³ is an aryl, a heterocyclyl, or a heteroaryl, R³ is optionally substituted with up to three substituents selected independently from a halogen, a hydroxyl, a cyano, an alkoxy having up to 12 carbon atoms, or R¹¹;
R⁴, in each occurrence, is selected independently from: 1) halogen, cyano, or hydroxy; 2) an alkyl, a cycloalkyl, a cycloalkoxy, an alkoxy, a haloalkyl, or a haloalkoxy, any of which having up to 12 carbon atoms; 3) a substituted or an unsubstituted aryl, aralkyl, aryloxy, heteroaryl, or heteroaryloxy, any of which having up to 12 carbon atoms, wherein any heteroaryl or heteroaryloxy comprises at least one heteroatom or heterogroup selected independently from O, N, S, or NR¹⁰; or 4) CO₂R⁶, COR⁸, SO₂R⁸, SO₂NR⁶R⁷, CONR⁶R⁷, or (CH₂)_{q}NR⁶R⁷, wherein q is an integer from 0 to 5, inclusive;
   m is an integer from 0 to 3, inclusive;
or R⁴ₙ, is a fused cyclic moiety comprising from 3 to 5 additional ring carbon atoms, inclusive, and optionally comprising at least one heteroatom or heterogroup selected independently from O, N, S, NR¹⁰, SO₂, or CO;
R⁵, in each occurrence, is selected independently from: 1) an alkoxy, a haloalkoxy, or a cycloalkyl, any of which having up to 12 carbon atoms; 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO; 3) hydroxyl, NR⁶R⁷, CO₂R⁶, COR⁸, or SO₂R⁸; or 4) a substituted or an unsubstituted heterocycloalkyl comprising from 3 to 7 ring carbon atoms, and from 1 to 3 heteroatoms or heterogroups, inclusive, selected independently from O, N, S, NR¹⁰, SO₂, or CO;
R⁶ and R⁷, in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
or R⁶ and R⁷ together with the nitrogen atom to which they are attached form a substituted or an unsubstituted cyclic moiety having from 3 to 7 ring carbon atoms, and optionally comprising 1, 2, or 3 heteroatoms in addition to the nitrogen atom to which R⁶ and R⁷ are bonded, selected independently from O, N, S, or NR¹⁰;
R⁸, in each occurrence, is selected independently from: 1) an alkyl, a cycloalkyl, or a haloalkyl, any of which having up to 12 carbon atoms; or 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
   Z is N or CH; or the ZR¹ moiety is S, CO, or SO₂; or the ZR¹R² moiety is —C≡CR²;
R¹¹ is selected independently from:
1) an alkyl, a haloalkyl, a cycloalkyl, or an alkoxycarbonyl, any of which having up to 12 carbon atoms;
2) a substituted or an unsubstituted heteroaryl or heterocyclyl, any of which having up to 12 carbon atoms, comprises at least one heteroatom or heterogroup selected independently from O, N, S, NR¹⁰, SO₂, or CO, wherein any substituted heteroaryl or heterocyclyl is substituted with up to three substituents selected independently from an alkyl having up to 12 carbon atoms or a hydroxyl; or
3) —CO—Z²—R¹³, —CO—R¹², —CO—Z²—(CH₂)ᵣ—CO—Z²—R¹³, —NR¹⁵R¹⁶, —Z²—CO—(CH₂)ᵣ—Z²—R¹³, —Z²—CO—(CH₂)ᵣ—CO—Z²—R¹³, —O—(CH₂)ᵣ—CO—Z²—R¹³, —O—(CH₂)ᵣ—R¹⁴, —O—R¹²—(CH₂)ᵣ—R¹³, —O—R¹⁴—CO—O—R¹³, —O—(CH₂)ᵣ—R¹², —O—(CH₂)ᵣ—NR′R″, —O—(CH₂)ᵣ—CO₂—(CH₂)ᵣ—R¹³, —O—(CH₂)ᵣ—SR⁸, —O—(CH₂)ᵣ—CO₂—R¹³, —O—(CH₂)ᵣ—CONR′R″, —O—(CH₂)ᵣ—CONH—(CH₂)ᵣ—OR¹³, —O—(CH₂)ᵣ—SO₂R⁸, —O—(CH₂)ᵣ—R¹³, —O—(CH₂)ᵣ—OR¹³, —O—(CH₂)ᵣ—O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″, —(CH₂)ᵣ—O—CO—R⁸, —(CH₂)ᵣ—R¹², —(CH₂)ᵣ—R¹³, —(CH₂)ᵣ—CO—Z²—R¹³, —(CH₂)—Z²—R¹³, or -alkenylene-CO₂—(CH₂)ᵣ—R¹³;
   r, in each occurrence, is independently 1, 2, or 3;
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl, any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
   Z², in each occurrence, is selected independently from NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹², —CO₂—(CH₂)ᵣ—CO—Z²—R¹³, —CO₂—(CH₂)ᵣ—OR¹³, —CO—(CH₂)ᵣ—O—(CH₂)ᵣ—O—(CH₂)ᵣ—R¹³, —CO—(CH₂)ᵣ—O(CH₂)ᵣ—OR¹³, or —CO—NH—(CH₂)ᵣ—OR¹³;
or R¹⁵ and R¹⁶ together with the nitrogen atom to which they are attached form a substituted or an unsubstituted cyclic moiety comprising up to 12 carbon atoms, optionally comprising at least one additional heteroatom or heterogroup selected independently from O, N, S, NR¹⁰, SO₂, or CO; wherein any substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰; or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂R⁸, —SO₂NR′R″, or —NR′R″;
wherein the —(CH₂)ᵣ— linking moiety, in any occurrence, is optionally substituted with at least one group selected independently from hydroxyl, amino, or an alkyl having up to 3 carbon atoms;
when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl, any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
when R¹ and R² together with the diradical Z to which they are attached_form a monocyclic or a bicyclic moiety, the cyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl or a heteroaryloxy, any of which having up to 12 carbon atoms, wherein any heteroaryl or heteroaryloxy comprises at least one heteroatom or heterogroup selected independently from O, N, S, or NR¹⁰; or 3) CO₂R⁶, COR⁸, SO₂R⁸, SO₂NR⁶R⁷, or CONR⁶R⁷;
R⁴, R⁶, R⁷, and R⁸ are optionally substituted with at least one substituent, and when substituted, the substituents are selected independently from: 1) halogen, hydroxy, cyano, or NR⁶R⁷; or 2) an alkyl or an alkoxy, any of which having up to 12 carbon atoms;

and R⁵ is optionally substituted with at least one substituent, and when substituted, the substituents are selected independently from: 1) halogen, hydroxy, cyano, or NR₆R₇; or 2) an alkyl having up to 12 carbon atoms.

In one embodiment of the above aspect, the present application relates to a pharmaceutical composition comprising
a) a CETP inhibitor having formula (Ia′),
b) at least one solubility improving material,
c) optionally one or more wetting agents, and
d) at least one pharmaceutically acceptable excipient; wherein formula (Ia′) is defined as follows

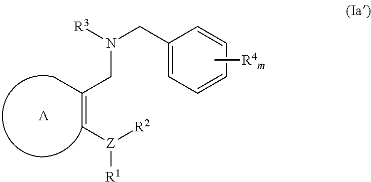

or a stereoisomer thereof or a pharmaceutically acceptable salt thereof, wherein A-ZR¹R² is:

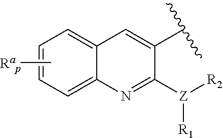

wherein R^{a}, in each occurrence, is selected independently from: 1) a hydrogen, a halogen, a cyano, or a hydroxyl; 2) an alkyl, a haloalkyl, a cycloalkyl, a (cycloalkyl)alkyl, an alkoxy, a cycloalkoxy, a haloalkoxy, an aryl, an aralkyl, a heteroaryl or a heterocyclyl, any of which having up to 12 carbon atoms, wherein any heteroaryl or heterocyclyl, comprises at least one heteroatom or heterogroup selected independently from O, N, S, NR¹⁰, SO₂, or CO; 3) CO₂R⁶, COR⁸, NR⁶R⁷ or SO₂R⁸;
   p is an integer from 0 to 3, inclusive;
Z is N or CH; or the ZR¹ moiety is S, SO, CO, or SO₂; or the ZR¹R² moiety is C≡CR² or —C(O)Z³R^{f}, wherein R^{f} is an alkyl, a cycloalkyl, or a (cycloalkyl)alkyl, any of which having up to 12 carbon atoms, or hydrogen; and Z³ is O or NR^{k}, wherein R^{k} is an alkyl, a cycloalkyl, or a (cycloalkyl)alkyl, any of which having up to 12 carbon atoms, or hydrogen;
R¹ and R² are selected independently from: 1) hydrogen; 2) an alkyl having up to 6 carbon atoms; 3) a cycloalkyl having up to 6 carbon atoms; 4) COR⁸; or 5) (CH₂)ₙR⁵ or (CH₂)ₙR^{d}CO₂R^{e}; wherein n, in each occurrence, is 1 or 2; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, NR¹⁰, SO₂, or CO; and R^{e}, in each occurrence, is selected independently from an alkyl or a cycloalkyl, either of which having up to 12 carbon atoms, or hydrogen;
or R¹ and R² together form a substituted or an unsubstituted monocyclic or bicyclic moiety comprising up to 12 carbon atoms, and optionally comprising 1 or 2 heteroatoms or heterogroups selected independently from O, N, or NR¹⁰; wherein any optional substituent on the cyclic moiety selected from: 1) a cycloalkyl having up to 6 carbon atoms; or 2) an alkyl having up to 2 carbon atoms;
R³ is selected from: 1) cyano; 2) a substituted or an unsubstituted alkyl having up to 12 carbon atoms; 3) a substituted or an unsubstituted aryl, or a substituted or an unsubstituted 5-, 6-, or 7-membered heterocyclyl or heteroaryl, comprising 1, 2, or 3 heteroatoms or heterogroups selected independently from O, N, S, NR¹⁰, SO₂, or CO; any of which having up to 12 carbon atoms; or 4) CO₂R⁶, COR⁸, SO₂R⁸, SO₂NR⁶R⁷, CONR⁶R⁷, C(S)NR⁶R⁷, C(═NH)OR⁸, C(S)NHC(O)OR⁸, or C(S)SR⁸; wherein when R³ is an alkyl, an aryl, a heterocyclyl, or a heteroaryl, R³ is optionally substituted with up to three substituents selected independently from R¹¹;
R⁴, in each occurrence, is selected independently from: 1) halogen, hydroxy or cyano; or 2) an alkyl, an alkoxy, a haloalkyl, or a haloalkoxy any of which having up to 4 carbon atoms; and m is an integer from 1-3, inclusive;
R⁵, in each occurrence, is selected independently from: 1) a substituted or an unsubstituted cycloalkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁶ and R², in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁸, in each occurrence, is selected independently from: 1) an alkyl, a cycloalkyl, or a haloalkyl, any of which having up to 12 carbon atoms; or 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
   R¹¹ is selected independently from:
1) a halogen, a hydroxyl or a cyano;
2) an alkyl, a haloalkyl, an alkoxy, a cycloalkyl, or an alkoxycarbonyl, any of which having up to 12 carbon atoms;
3) a substituted or an unsubstituted heteroaryl or heterocyclyl, any of which having up to 12 carbon atoms, comprises at least one heteroatom or heterogroup selected independently from O, N, S, NR¹⁰, SO₂, or CO, wherein any substituted heteroaryl or heterocyclyl is substituted with up to three substituents selected independently from an alkyl having up to 12 carbon atoms or a hydroxyl; or
4) —CO—Z²—R¹³, —CO—R¹², —CO—Z²—(CH₂)ᵣ—CO—Z²—R¹³, —NR¹⁵R¹⁶,

—Z²—CO—(CH₂)ᵣ—Z²—R¹³, —Z²—CO—(CH₂)ᵣ—CO—Z²—R¹³, —O—(CH₂)ᵣ—CO—Z²—R¹³,

—O—(CH₂)ᵣ—R¹⁴, —O—R¹²—(CH₂)ᵣ—R¹³, —O—R¹⁴—CO—O—R¹³, —O—(CH₂)ᵣ—R¹²,

—O—(CH₂)ᵣ—NR′R″, —O—(CH₂)ᵣ—CO₂—(CH₂)ᵣ—R¹³, —O—(CH₂)ᵣ—SR⁸, —O—(CH₂)ᵣ—CO₂—R¹³,

—O—(CH₂)ᵣ—O—(CH₂)ᵣ—OR¹³,

—O—(CH₂)ᵣ—CONR′R″, —O—(CH₂)ᵣ—CONH—(CH₂)ᵣ—OR¹³, —O—(CH₂)ᵣ—SO₂R⁸,

—O—(CH₂)ᵣ—R¹³, —O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″,

—(CH₂)ᵣ—O—CO—R⁸, —(CH₂)ᵣ—R¹², —(CH₂)ᵣ—R¹³, —(CH₂)ᵣ—NH—(CH₂)ᵣ—OR¹³,

—(CH₂)ᵣ—CO—Z²—R¹³, —(CH₂)ᵣ—Z²—R¹³, —(CH₂)ᵣ—NH—CO—Z²—R¹³, or -alkenylene-CO₂—(CH₂)ᵣ—R¹³;
r, in each occurrence, is independently 1, 2, or 3;
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl, any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
   Z², in each occurrence, is selected independently from NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹², —CO₂—(CH₂)ᵣ—CO—Z²—R¹³, —CO₂—(CH₂)ᵣ—OR¹³, —CO—(CH₂)—O—(CH₂)—O—(CH₂)ᵣ—R¹³, —CO—(CH₂)ᵣ—O(CH₂)ᵣ—OR¹³, or —CO—NH—(CH₂)ᵣ—OR¹³;
or R¹⁵ and R¹⁶ together form a substituted or an unsubstituted cyclic moiety comprising up to 12 carbon atoms, optionally comprising at least one additional heteroatom or heterogroup selected independently from O, N, S, NR¹⁰, SO₂, or CO; wherein any substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰; or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂R⁸, —SO₂NR′R″, or —NR′R″; and
wherein the —(CH₂)ᵣ— linking moiety, in any occurrence, is optionally substituted with at least one group selected independently from hydroxyl, amino, or an alkyl having up to 3 carbon atoms;
wherein when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl, any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
wherein when R¹ and R² together form a monocyclic or a bicyclic moiety, the monocyclic or bicyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl or a heteroaryloxy, any of which having up to 12 carbon atoms, wherein any heteroaryl or heteroaryloxy comprises at least one heteroatom or heterogroup selected independently from O, N, S, or NR¹⁰; or 3) CO₂R⁶, (CH₂)_{q}COR⁸, SO₂R⁸, SO₂NR⁶R⁷, or CONR⁶R⁷; or 4) (CH₂)_{q}CO₂(CH₂)_{q}CH₃, wherein q is selected independently from an integer from 0 to 3, inclusive; and
R⁴, R⁶, R⁷, and R⁸ are optionally substituted with at least one substituent, and when substituted, the substituents are selected independently from: 1) halogen, hydroxy, cyano, or NR⁶R⁷; or 2) an alkyl or an alkoxy, any of which having up to 12 carbon atoms; and
R⁵ is optionally substituted with at least one substituent selected independently from: 1) halogen, hydroxy, cyano, or NR⁶R⁷; or 2) an alkyl or an alkoxy, any of which having up to 12 carbon atoms; or 3) (CH₂)ₜOR^{j} or (CH₂)ₜCOOR^{j} wherein t is an integer from 1 to 3, inclusive, and R is hydrogen or alkyl having up to 12 carbon atoms.

In another aspect, the present application relates to a pharmaceutical composition comprising
a) a CETP inhibitor having formula (II),
b) at least one solubility improving material,
c) optionally one or more wetting agents, and
d) at least one pharmaceutically acceptable excipient; wherein formula (II) is defined as follows

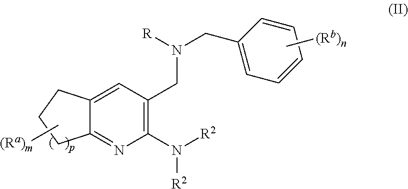

or a stereoisomer thereof or a pharmaceutically acceptable salt thereof; wherein, R represents

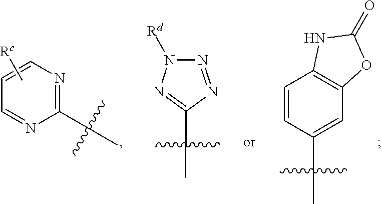

R¹ and R² are independently selected from hydrogen, acyl, haloalkyl, —(CHR^{e})_{q}R³, an optionally substituted group selected from alkyl or cycloalkyl, wherein optional substituent, in each occurrence, is independently selected from halogen, cyano, hydroxyl, an alkyl, a haloalkyl or an alkoxy;
R³ is a group selected from alkoxy, haloalkoxy, cycloalkyl, aryl, heterocyclyl or heteroaryl, wherein R³ is optionally substituted with a group selected from halogen, cyano, hydroxyl, alkyl, haloalkyl or alkoxy;
R^{a}, in each occurrence, is independently selected from halogen, cyano, hydroxy, alkyl, haloalkyl or alkoxy;
R^{b}, in each occurrence, is independently selected from halogen, alkyl, haloalkyl, hydroxy, alkoxy or haloalkoxy;
R^{c} is independently selected from hydrogen, cyano, halogen, —C(═O)—R^{f}, —CONR^{g}R^{h}, —C(═O)—CH≡CH—NRⁱR^{j}, an optionally substituted group selected from cycloalkyl, aryl, heteroaryl or heterocyclyl ring, wherein the optional substituent, in each occurrence, is selected independently from hydrogen, halogen, cyano, hydroxyl, alkyl, haloalkyl, alkoxy, alkoxyalkyl or haloalkoxy;
R^{d} is hydrogen or alkyl;
R^{e}, in each occurrence, is independently selected from hydrogen, alkyl or alkoxy;
R^{f} is hydrogen or alkyl;
R^{g} and R^{h} independently represent hydrogen or alkyl;
Rⁱ and R^{j} independently represent hydrogen or alkyl;
m is 0, 1 or 2;
n is 0, 1, 2 or 3;
p is 1 or 2; and
q is 0, 1, 2, 3, 4 or 5.

In yet another aspect, the present application relates to a pharmaceutical composition comprising
a) a CETP inhibitor having formula (III),
b) at least one solubility improving material,
c) optionally one or more wetting agents, and
d) at least one pharmaceutically acceptable excipient; wherein formula (III) is defined as follows

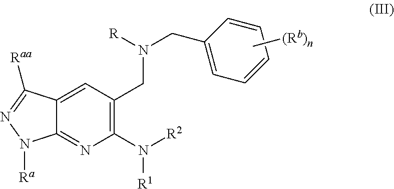

or a stereoisomer thereof or a pharmaceutically acceptable salt thereof, wherein, R represents hydrogen or

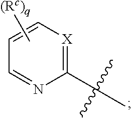

X represents CH or N;
R¹ and R² are independently of each other selected from hydrogen, acyl, alkyl or —(CH₂)ₚ-cycloalkyl;
R^{a} and R^{aa} are independently of each other selected from hydrogen or alkyl;
R^{b}, in each occurrence, is independently selected from halogen, alkyl, haloalkyl, hydroxy, alkoxy or haloalkoxy;
R^{c}, in each occurrence, is independently selected from hydrogen, cyano, halogen, alkyl, alkoxy, haloalkoxy, —COOR^{d}, —C(═O)—R^{e}, —CONR^{g}R^{h}, —C(═O)—CH═CH—NRⁱR^{j}, —NHCOR^{t}, an optionally substituted group selected from cycloalkyl, aryl, heteroaryl or heterocycle ring, wherein the optional substituent, in each occurrence, is selected independently from hydrogen, halogen, cyano, hydroxyl, alkyl, haloalkyl, alkoxy, alkoxyalkyl or haloalkoxy;
R^{d}, R^{e}, R^{g}, R^{h}, Rⁱ and R^{j}, in each occurrence, independently of each other represents hydrogen or alkyl;
R^{t} is selected from hydrogen, alkyl or cycloalkyl;
n is 0, 1, 2 or 3;
p is 0, 1, or 2; and
q is 1 or 2.

In another embodiment, the application provides pharmaceutical compositions comprising one or more specific compounds of formulae (I), (Ia′), (II) or (III) and is enumerated as follows:

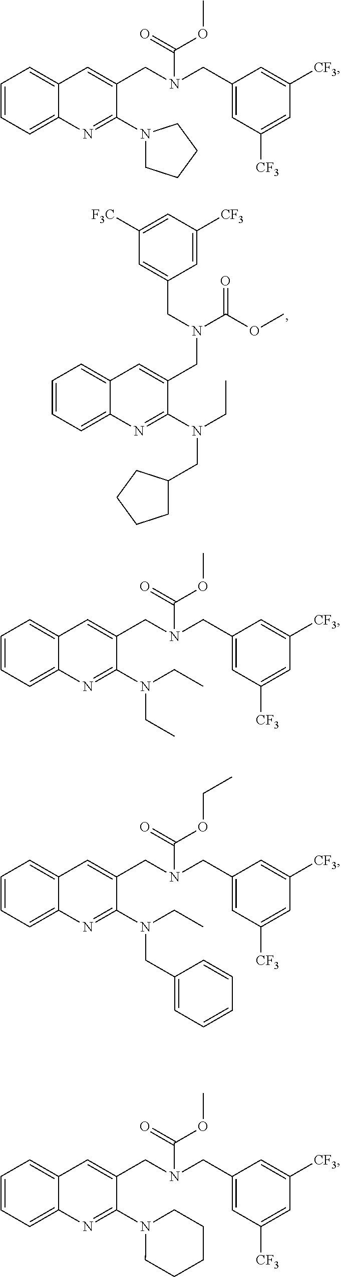

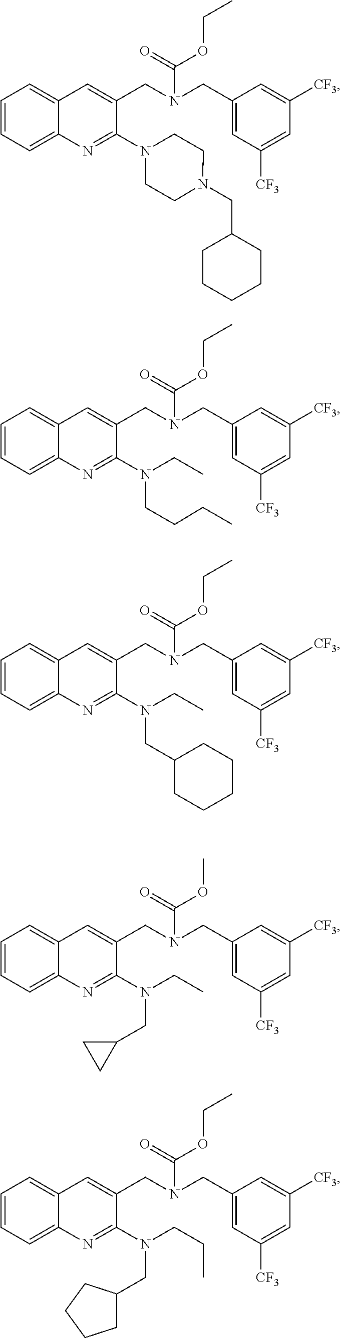

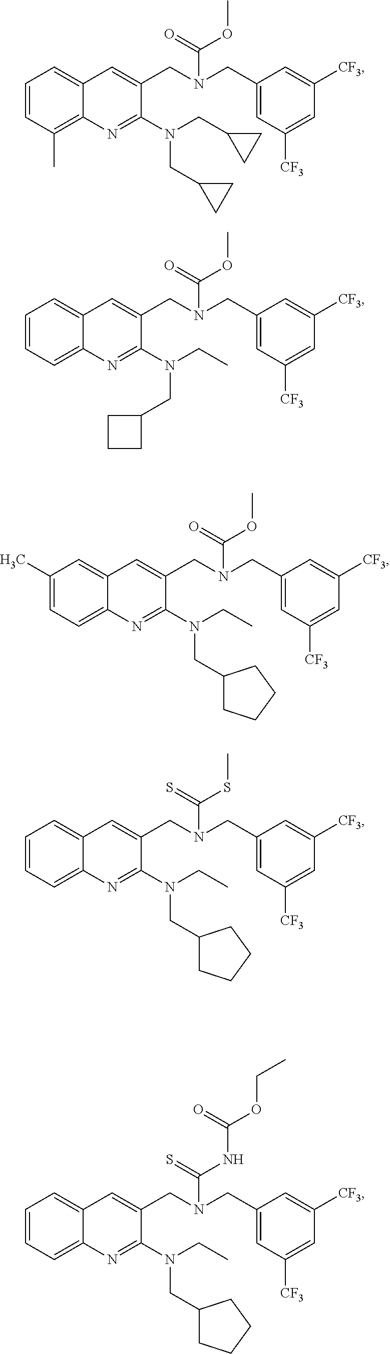

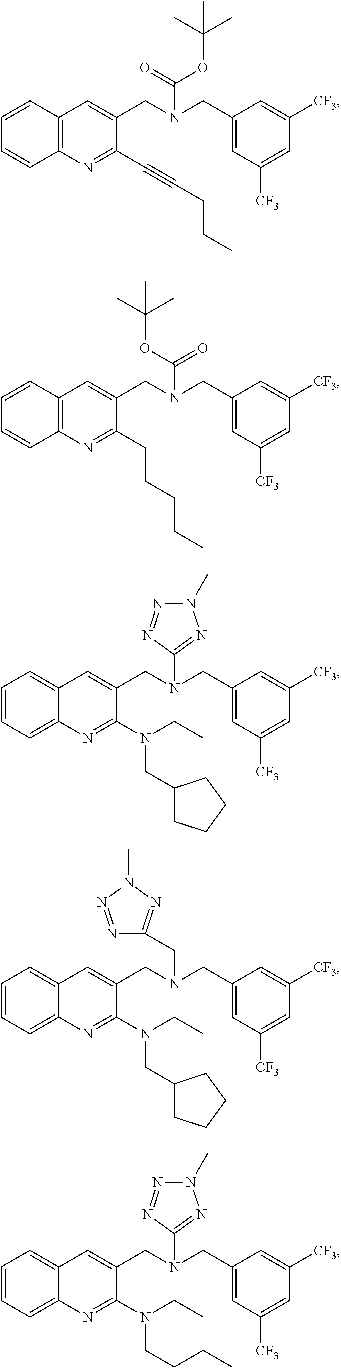

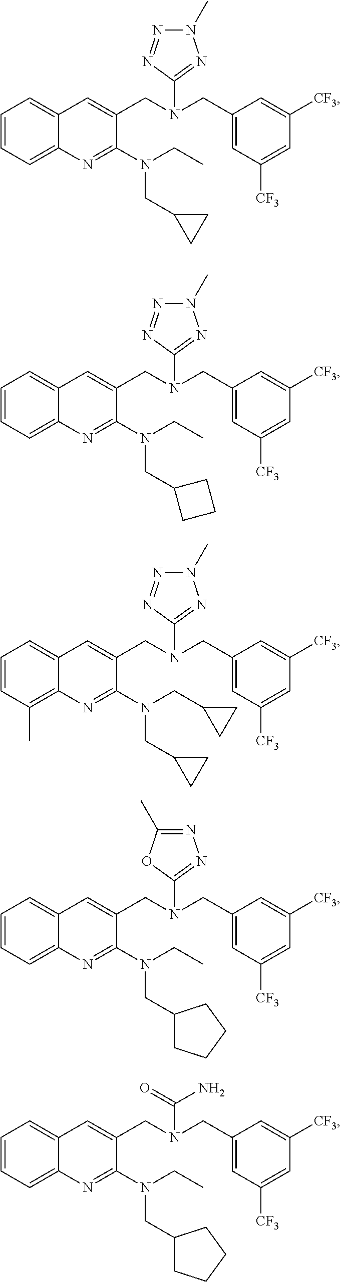

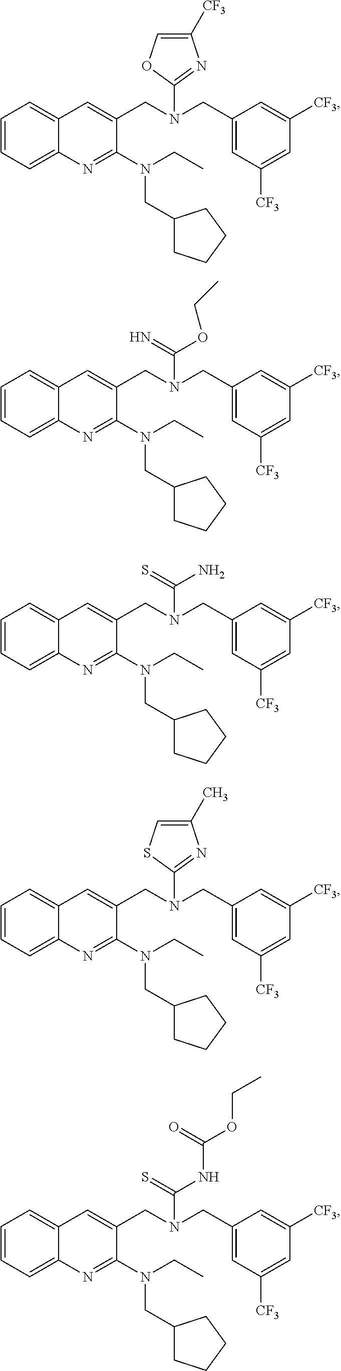

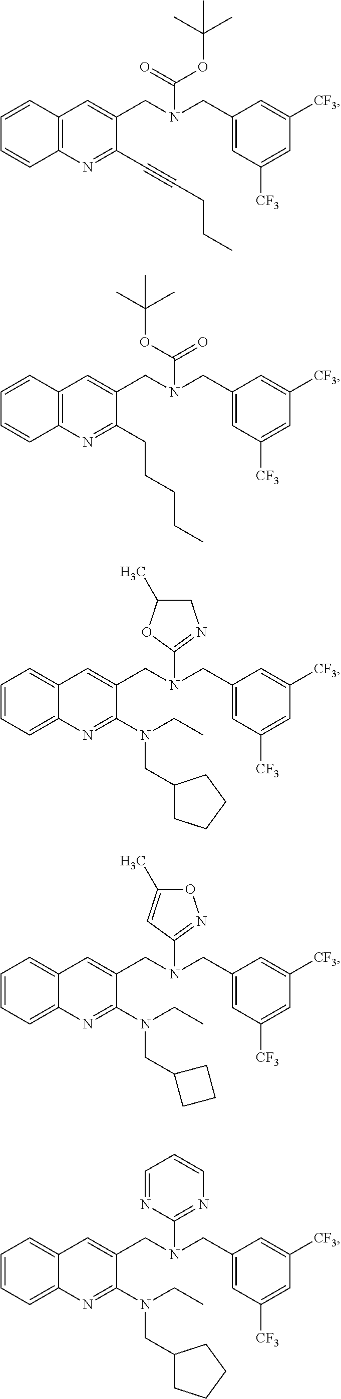 [SMILES: CCCC#Cc1nc2ccccc2cc1CN(Cc1cc(C(F)(F)F)cc(C(F)(F)F)c1)C(=O)OC(C)(C)C ; CCCCCc1nc2ccccc2cc1CN(Cc1cc(C(F)(F)F)cc(C(F)(F)F)c1)C(=O)OC(C)(C)C ; CCN(CC1CCCC1)c1nc2ccccc2cc1CN(Cc1cc(C(F)(F)F)cc(C(F)(F)F)c1)C1=NCC(C)O1 ; CCN(CC1CCC1)c1nc2ccccc2cc1CN(Cc1cc(C(F)(F)F)cc(C(F)(F)F)c1)c1cc(C)on1 ; CCN(CC1CCCC1)c1nc2ccccc2cc1CN(Cc1cc(C(F)(F)F)cc(C(F)(F)F)c1)c1ncccn1]

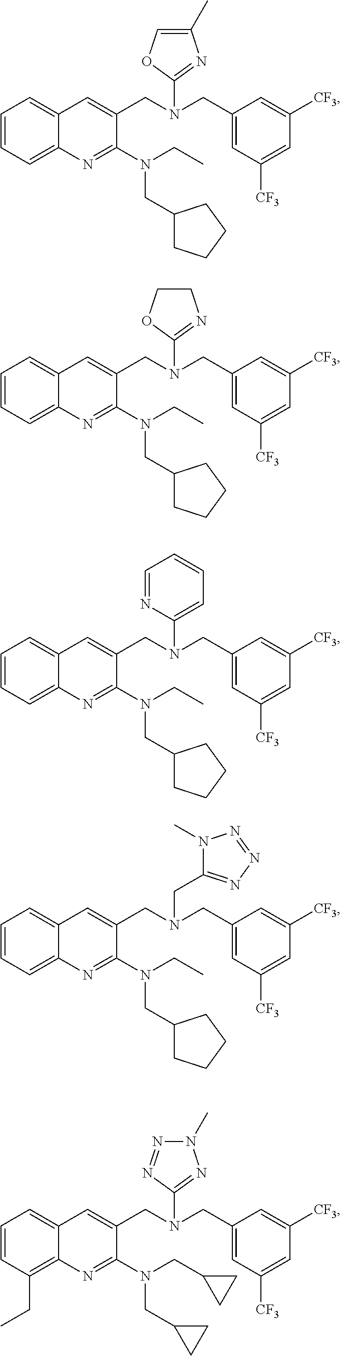

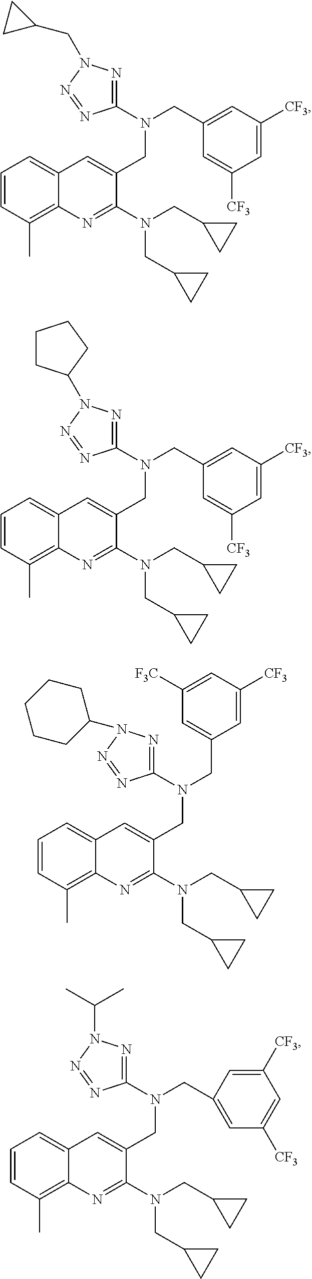

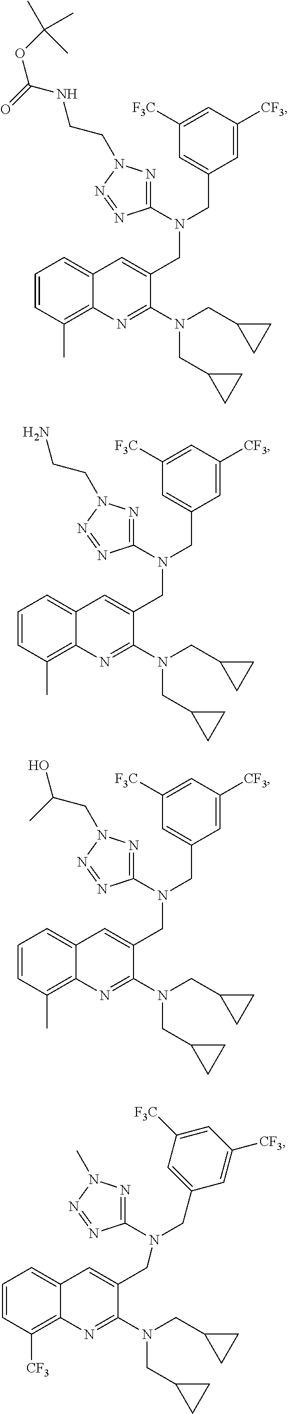

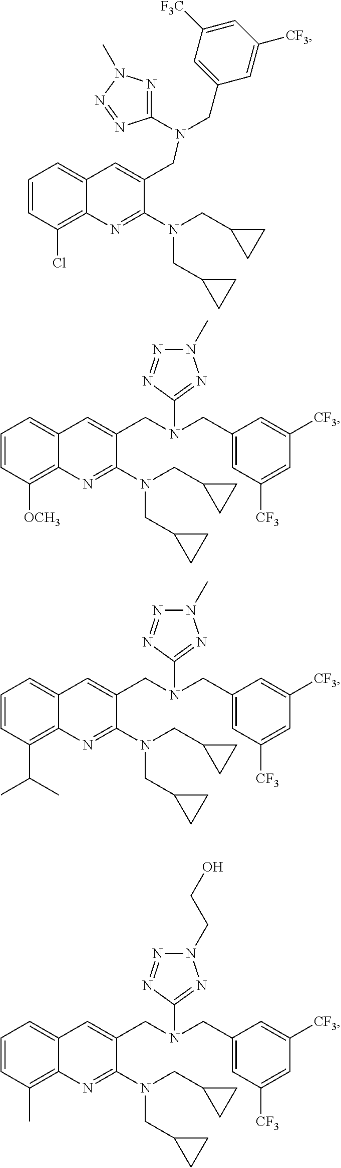

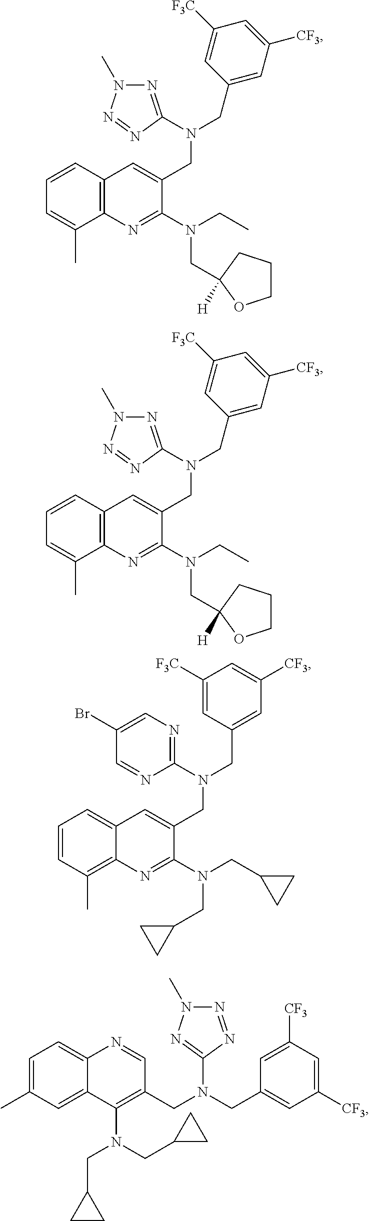

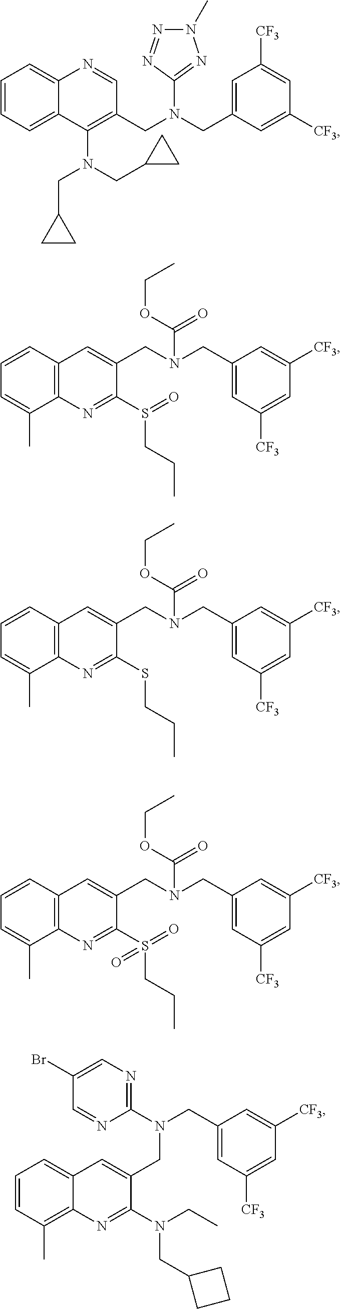

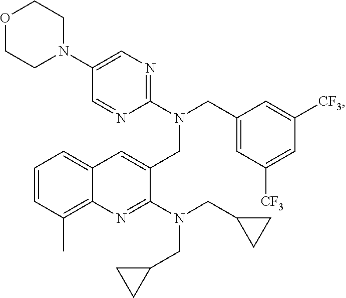 [SMILES: Cc1cccc2cc(CN(Cc3cc(C(F)(F)F)cc(C(F)(F)F)c3)c3ncc(N4CCOCC4)cn3)c(N(CC3CC3)CC3CC3)nc12]

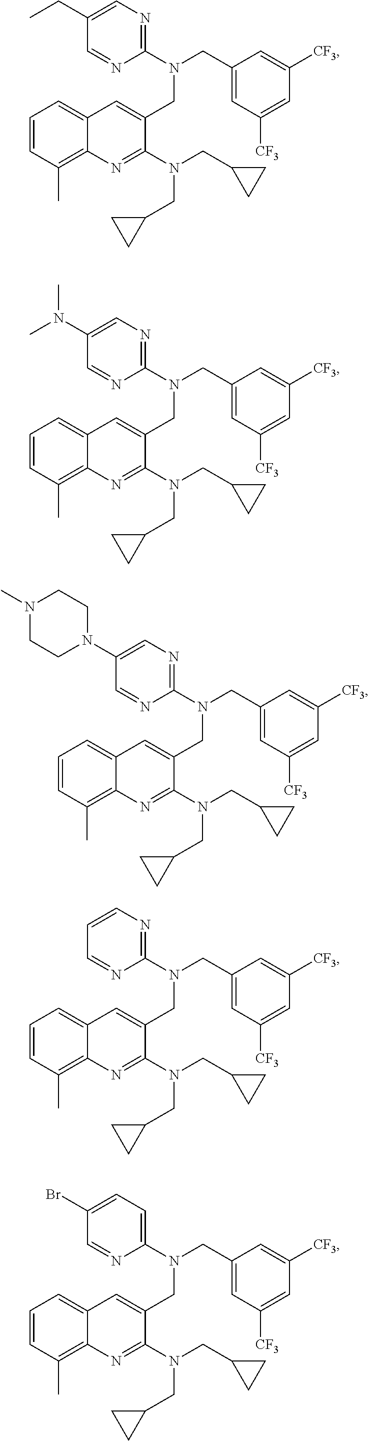

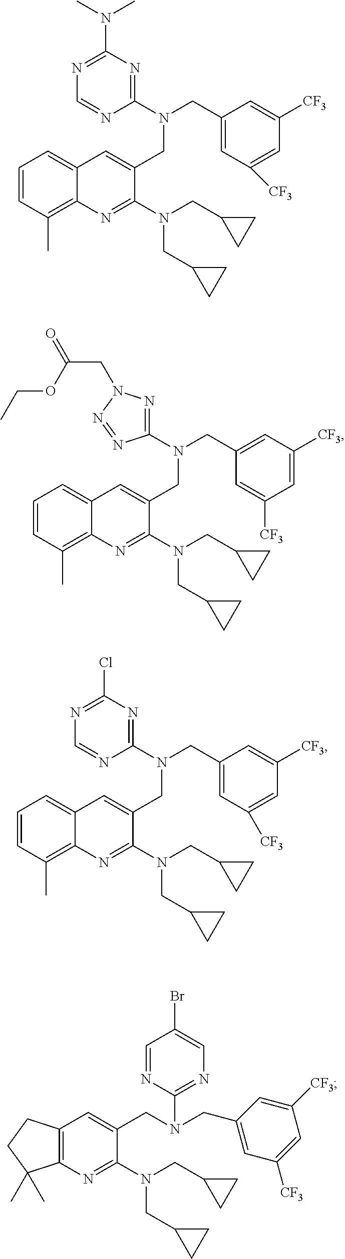 [SMILES: Cc1cccc2cc(CN(Cc3cc(C(F)(F)F)cc(C(F)(F)F)c3)c3ncnc(N(C)C)n3)c(N(CC3CC3)CC3CC3)nc12 ; CCOC(=O)Cn1nnc(N(Cc2cc(C(F)(F)F)cc(C(F)(F)F)c2)Cc2cc3cccc(C)c3nc2N(CC2CC2)CC2CC2)n1 ; Cc1cccc2cc(CN(Cc3cc(C(F)(F)F)cc(C(F)(F)F)c3)c3ncnc(Cl)n3)c(N(CC3CC3)CC3CC3)nc12 ; CC1(C)CCc2cc(CN(Cc3cc(C(F)(F)F)cc(C(F)(F)F)c3)c3ncc(Br)cn3)c(N(CC3CC3)CC3CC3)nc21]

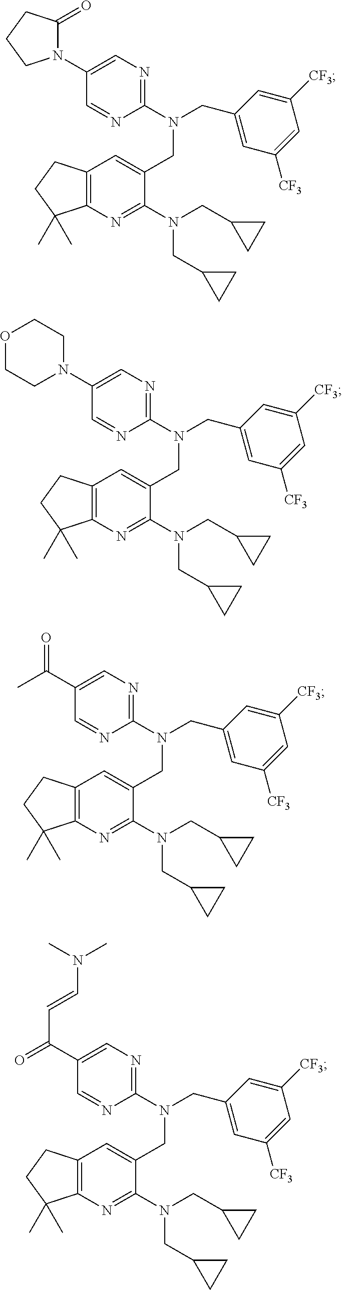 [SMILES: CC1(C)CCc2cc(CN(Cc3cc(C(F)(F)F)cc(C(F)(F)F)c3)c3ncc(N4CCCC4=O)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; CC(=O)c1cnc(N(Cc2cc(C(F)(F)F)cc(C(F)(F)F)c2)Cc2cc3c(nc2N(CC2CC2)CC2CC2)C(C)(C)CC3)nc1 ; CN(C)/C=C/C(=O)c1cnc(N(Cc2cc(C(F)(F)F)cc(C(F)(F)F)c2)Cc2cc3c(nc2N(CC2CC2)CC2CC2)C(C)(C)CC3)nc1]

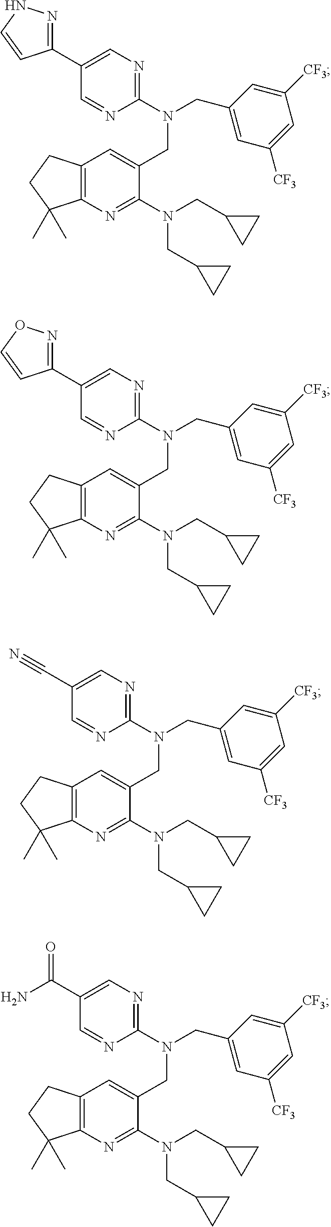 [SMILES: CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(-c4ccon4)cn3)c(N(CC3CC3)CC3CC3)nc21 ; CC1(C)CCc2cc(CN(Cc3cc(C(F)(F)F)cc(C(F)(F)F)c3)c3ncc(C#N)cn3)c(N(CC3CC3)CC3CC3)nc21 ; CC1(C)CCc2cc(CN(Cc3cc(C(F)(F)F)cc(C(F)(F)F)c3)c3ncc(C(N)=O)cn3)c(N(CC3CC3)CC3CC3)nc21]

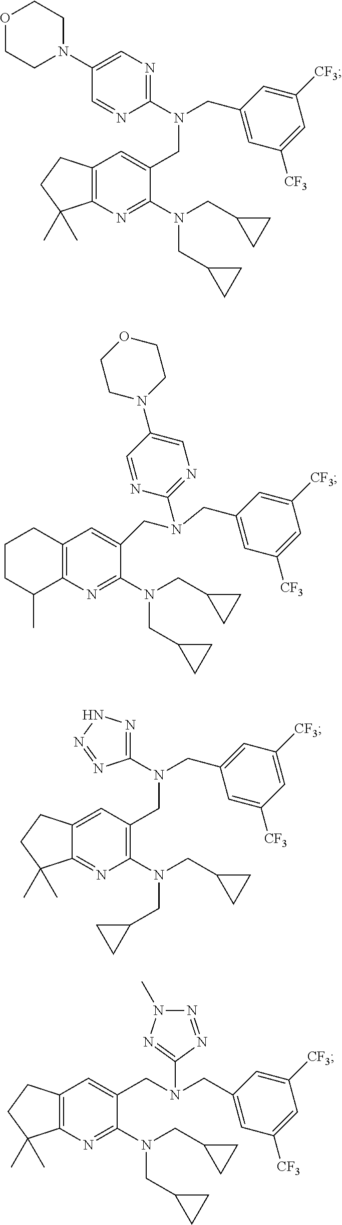

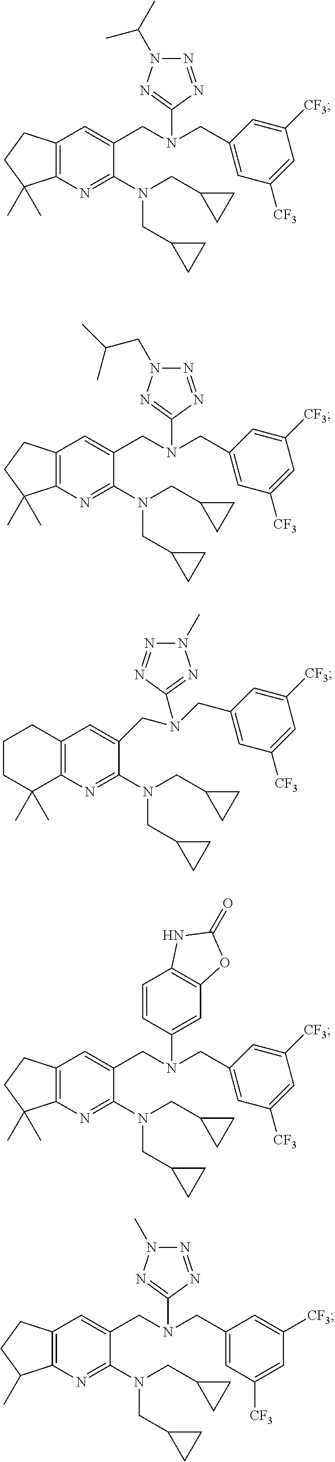

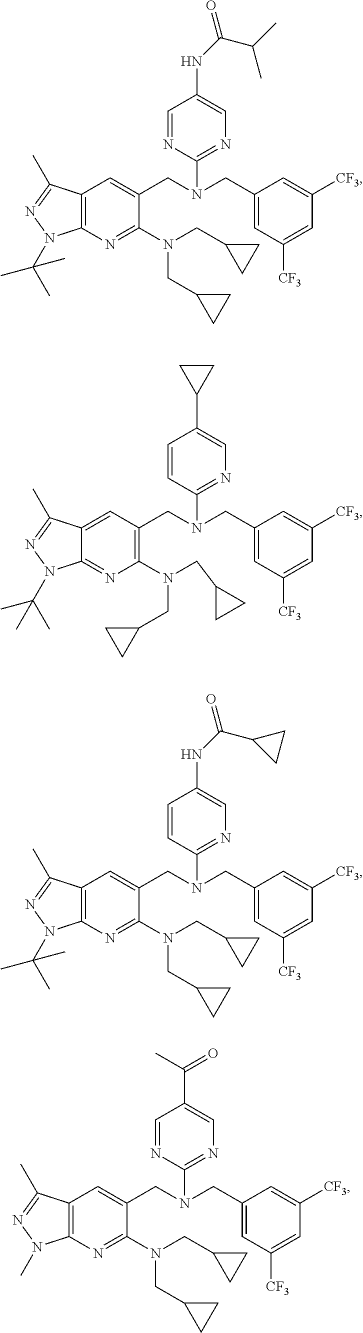

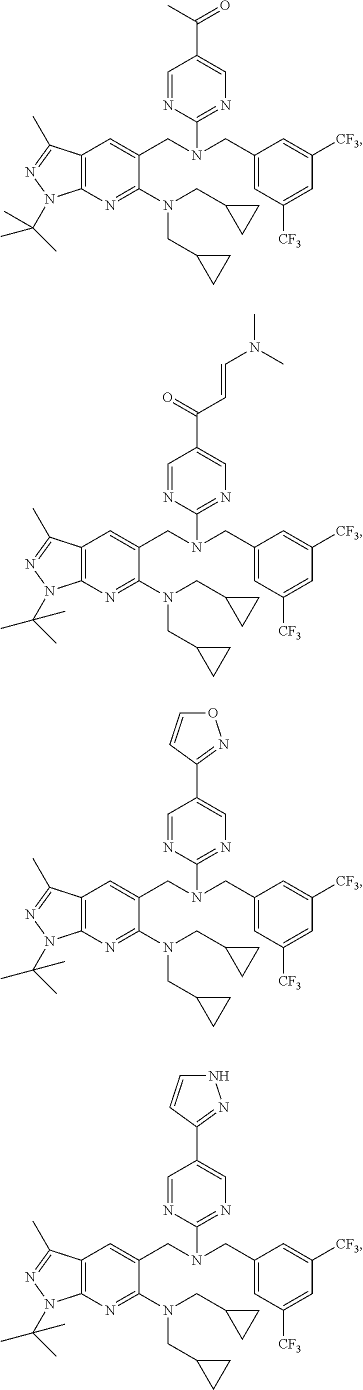

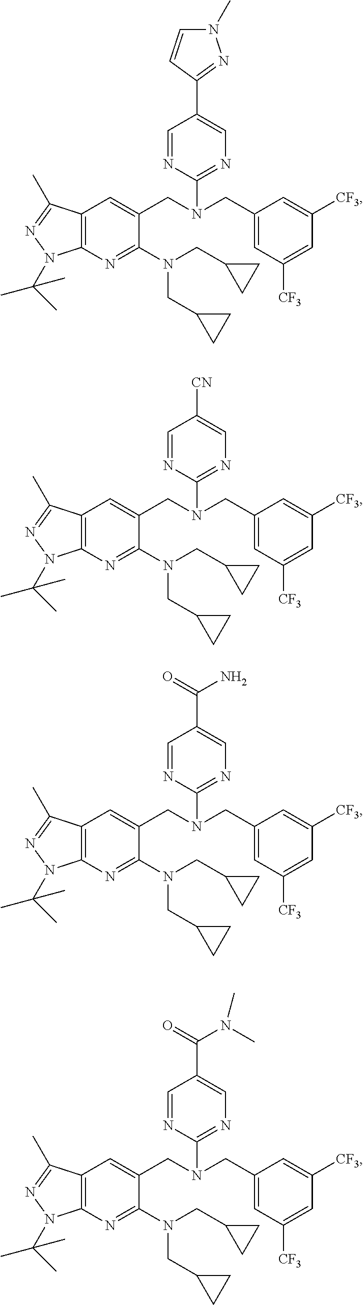

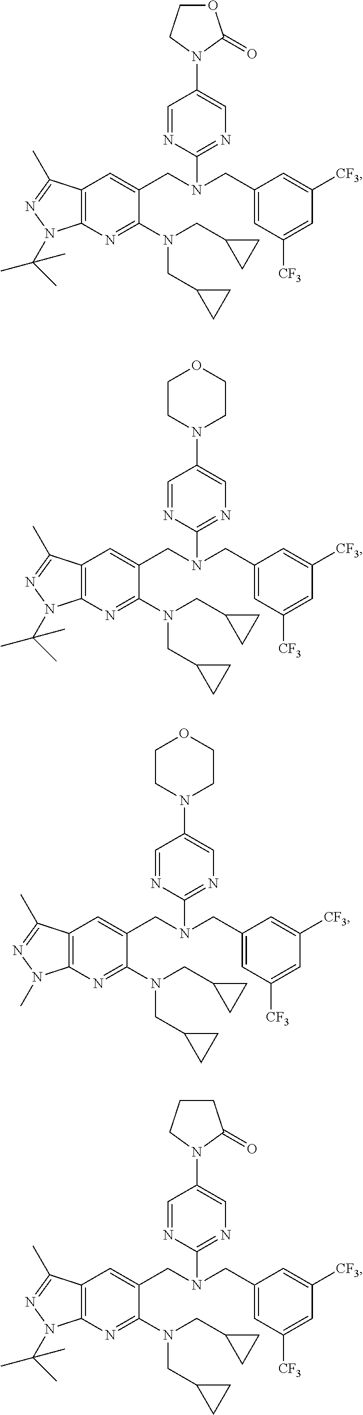

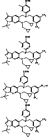

or a stereoisomer thereof or a pharmaceutically acceptable salt thereof.

In one embodiment, the application provides pharmaceutical compositions comprising one or more specific compounds of formula (I) and are enumerated as follows:

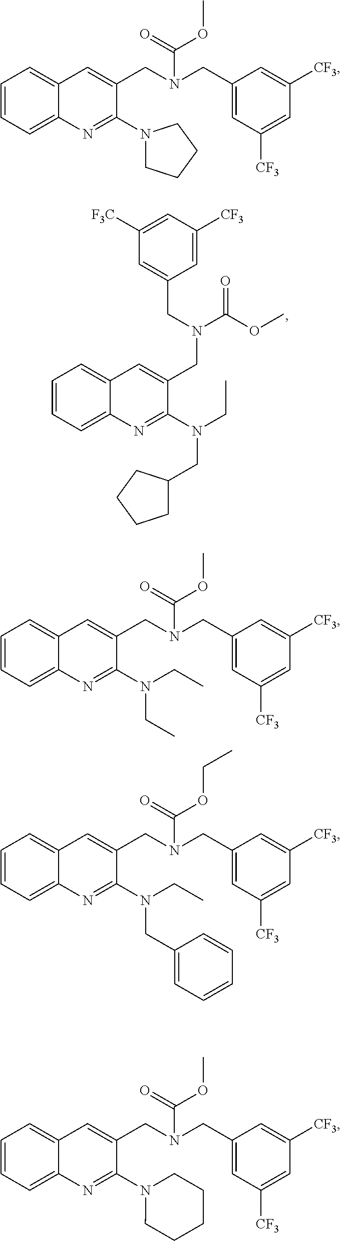

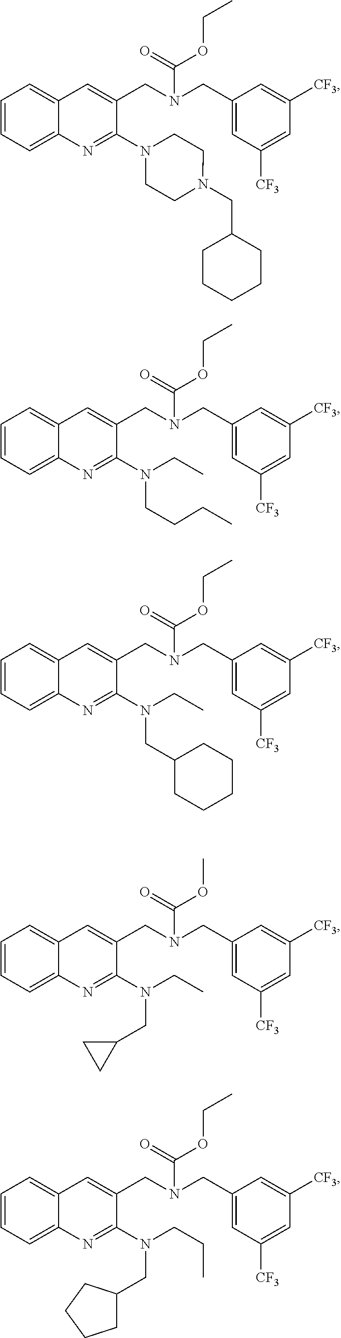

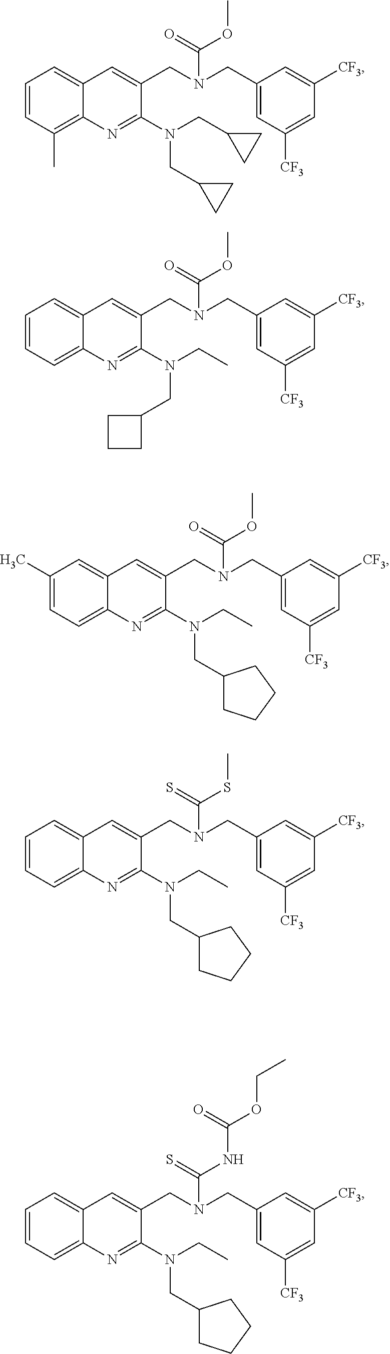

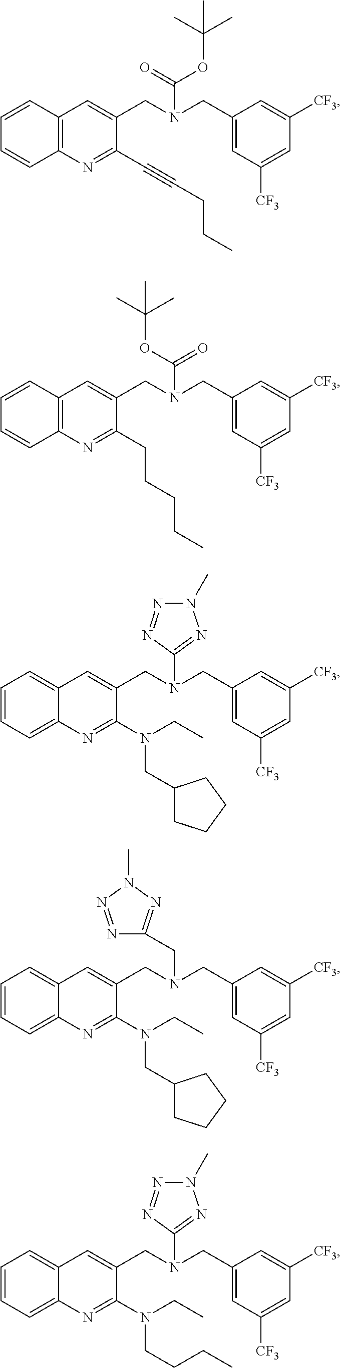

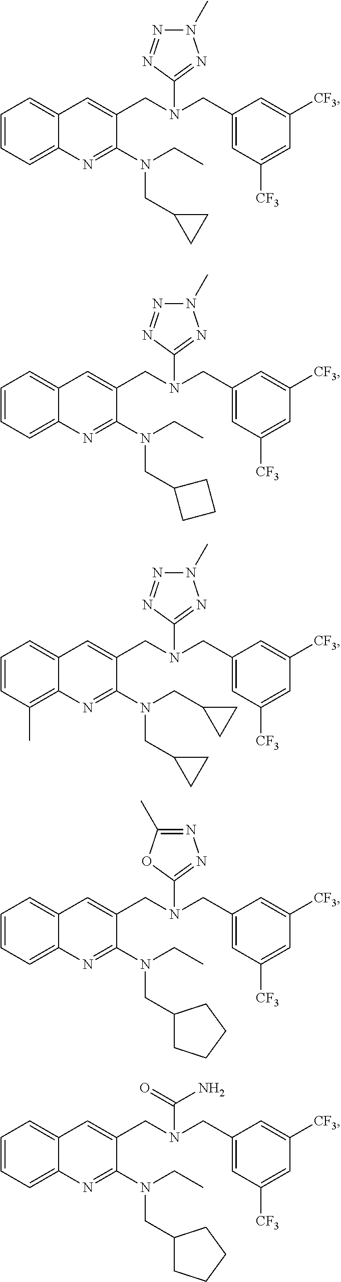

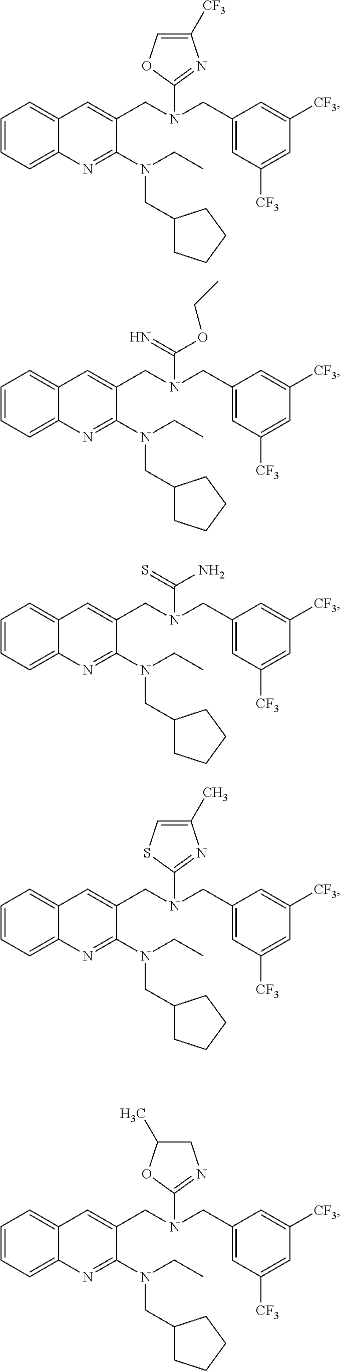

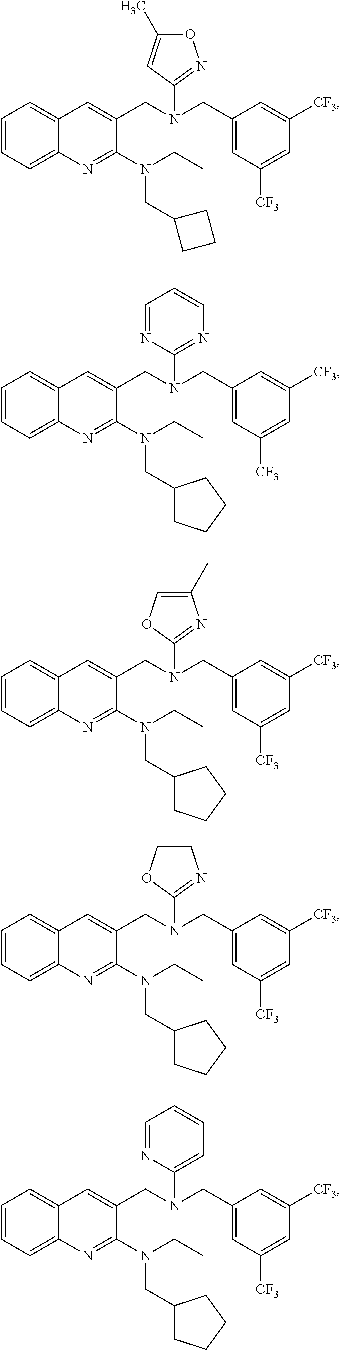

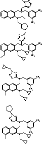

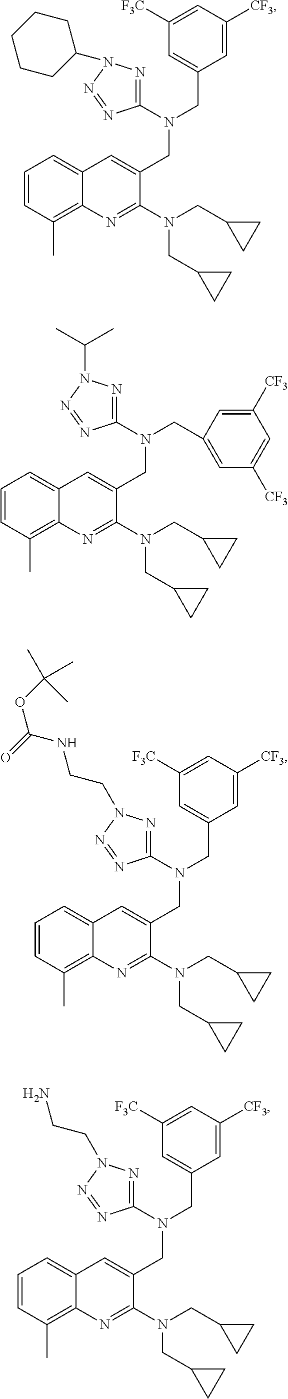 [SMILES: Cc1cccc2cc(CN(Cc3cc(C(F)(F)F)cc(C(F)(F)F)c3)c3nnn(C4CCCCC4)n3)c(N(CC3CC3)CC3CC3)nc12 ; Cc1cccc2cc(CN(Cc3cc(C(F)(F)F)cc(C(F)(F)F)c3)c3nnn(C(C)C)n3)c(N(CC3CC3)CC3CC3)nc12 ; Cc1cccc2cc(CN(Cc3cc(C(F)(F)F)cc(C(F)(F)F)c3)c3nnn(CCNC(=O)OC(C)(C)C)n3)c(N(CC3CC3)CC3CC3)nc12 ; Cc1cccc2cc(CN(Cc3cc(C(F)(F)F)cc(C(F)(F)F)c3)c3nnn(CCN)n3)c(N(CC3CC3)CC3CC3)nc12]

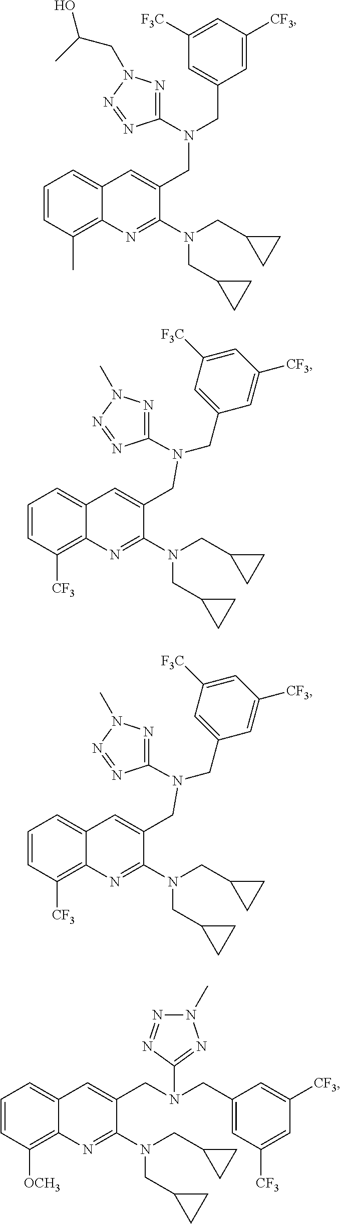

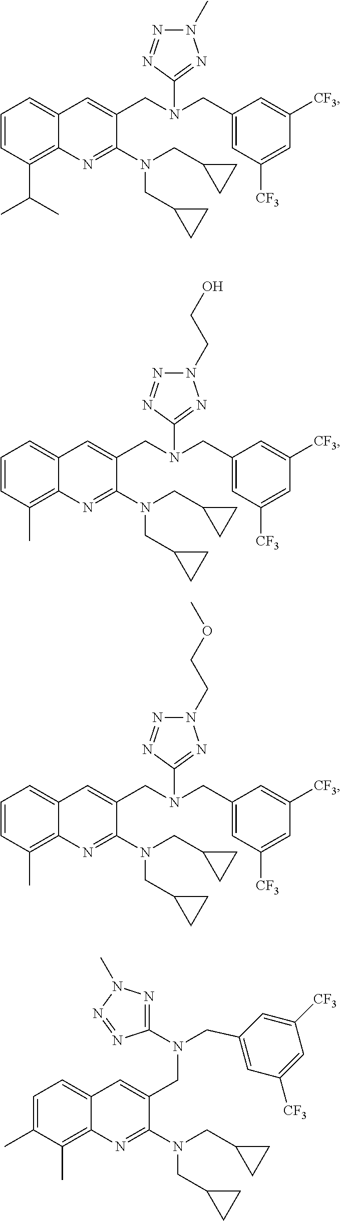

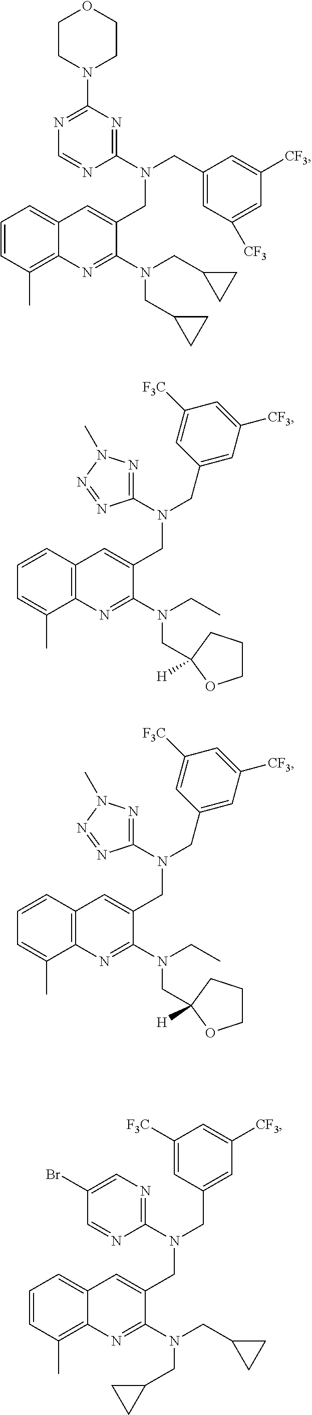 [SMILES: Cc1cccc2cc(CN(Cc3cc(C(F)(F)F)cc(C(F)(F)F)c3)c3ncnc(N4CCOCC4)n3)c(N(CC3CC3)CC3CC3)nc12 ; CCN(C[C@H]1CCCO1)c1nc2c(C)cccc2cc1CN(Cc1cc(C(F)(F)F)cc(C(F)(F)F)c1)c1nnn(C)n1 ; CCN(C[C@@H]1CCCO1)c1nc2c(C)cccc2cc1CN(Cc1cc(C(F)(F)F)cc(C(F)(F)F)c1)c1nnn(C)n1 ; Cc1cccc2cc(CN(Cc3cc(C(F)(F)F)cc(C(F)(F)F)c3)c3ncc(Br)cn3)c(N(CC3CC3)CC3CC3)nc12]

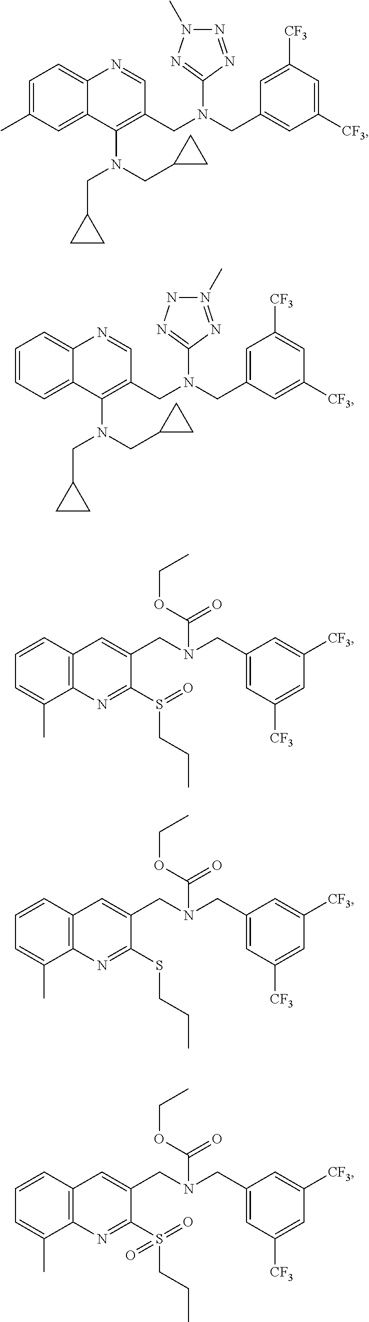

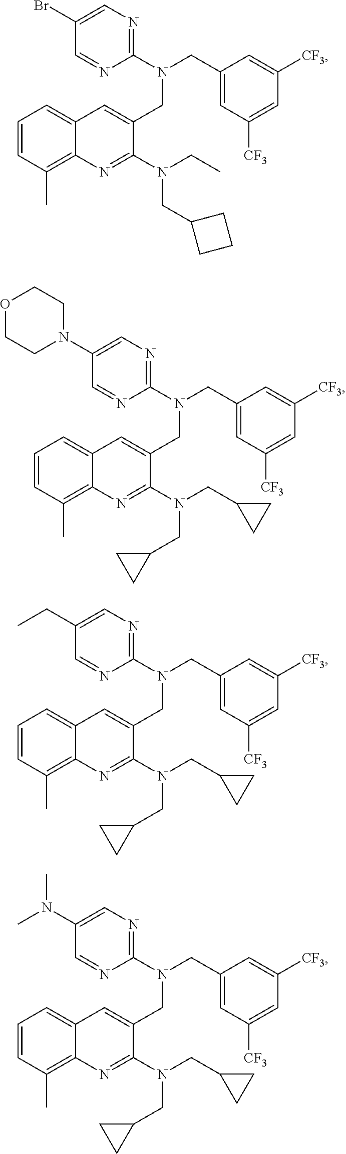

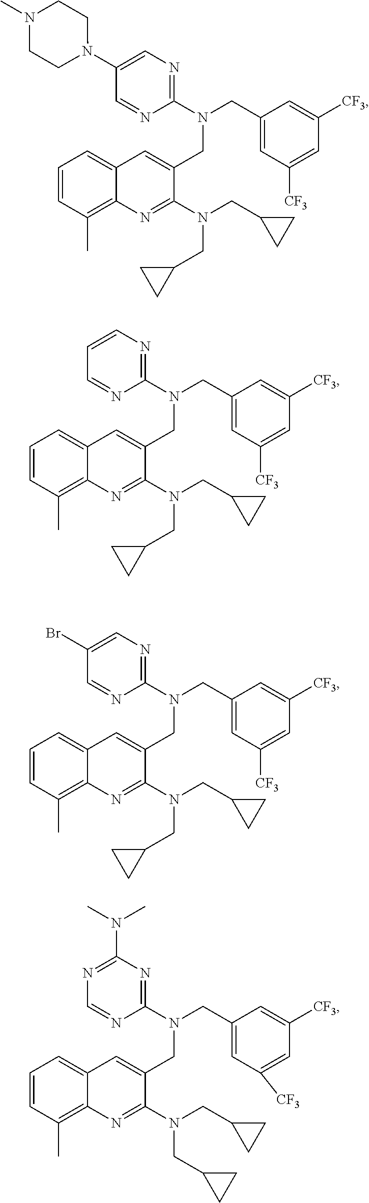

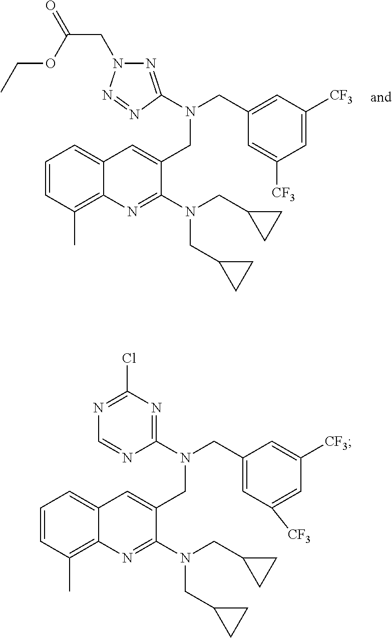 [SMILES: CCOC(=O)Cn1nnc(N(Cc2cc(C(F)(F)F)cc(C(F)(F)F)c2)Cc2cc3cccc(C)c3nc2N(CC2CC2)CC2CC2)n1 ; Cc1cccc2cc(CN(Cc3cc(C(F)(F)F)cc(C(F)(F)F)c3)c3ncnc(Cl)n3)c(N(CC3CC3)CC3CC3)nc12]

or a stereoisomer thereof or a pharmaceutically acceptable salt thereof.

In one embodiment, the application provides pharmaceutical compositions comprising one or more specific compounds of formula (II) and is enumerated as follows:

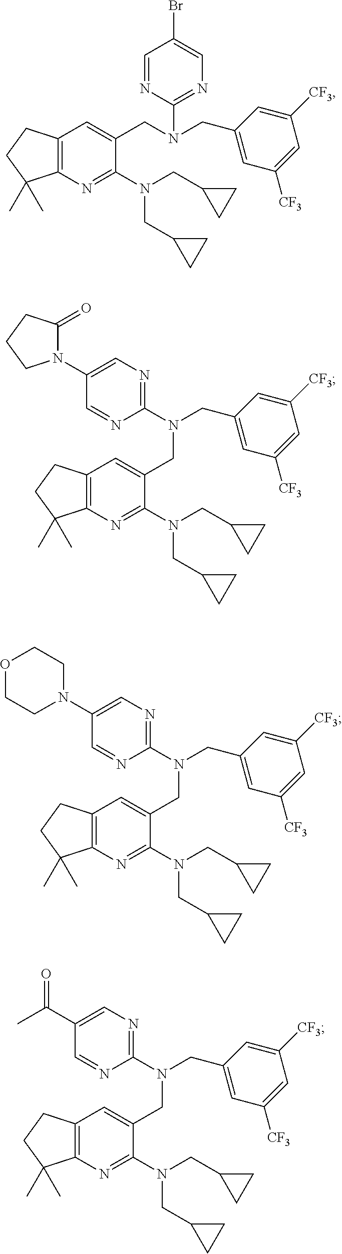 [SMILES: CC1(C)CCc2cc(CN(Cc3cc(C(F)(F)F)cc(C(F)(F)F)c3)c3ncc(Br)cn3)c(N(CC3CC3)CC3CC3)nc21 ; CC1(C)CCc2cc(CN(Cc3cc(C(F)(F)F)cc(C(F)(F)F)c3)c3ncc(N4CCCC4=O)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; CC(=O)c1cnc(N(Cc2cc(C(F)(F)F)cc(C(F)(F)F)c2)Cc2cc3c(nc2N(CC2CC2)CC2CC2)C(C)(C)CC3)nc1]

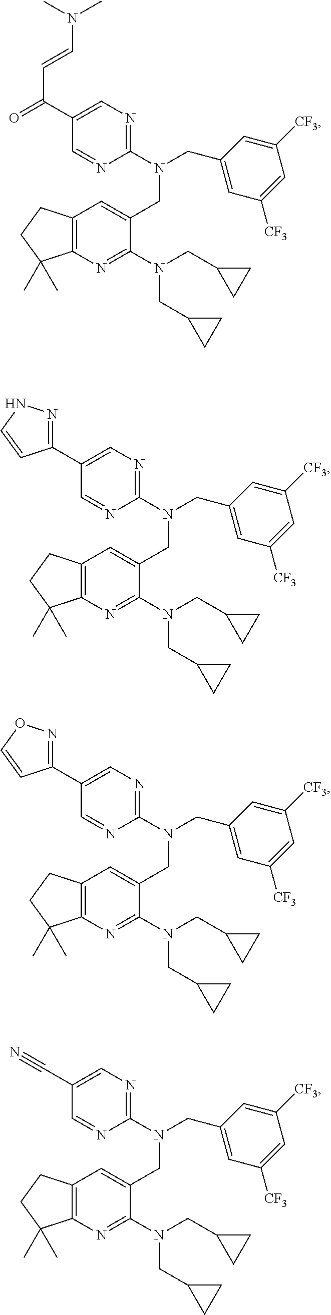 [SMILES: CN(C)/C=C/C(=O)c1cnc(N(Cc2cc(C(F)(F)F)cc(C(F)(F)F)c2)Cc2cc3c(nc2N(CC2CC2)CC2CC2)C(C)(C)CC3)nc1 ; CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(-c4ccon4)cn3)c(N(CC3CC3)CC3CC3)nc21 ; CC1(C)CCc2cc(CN(Cc3cc(C(F)(F)F)cc(C(F)(F)F)c3)c3ncc(C#N)cn3)c(N(CC3CC3)CC3CC3)nc21]

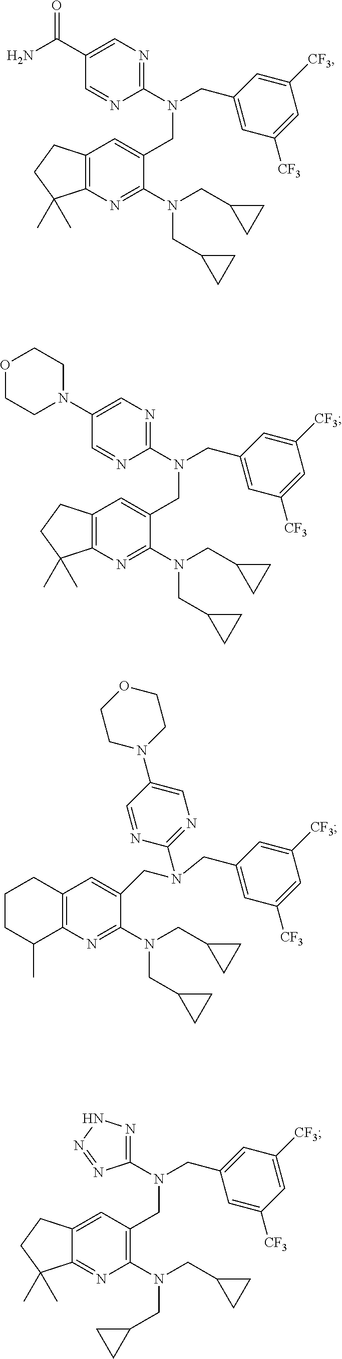 [SMILES: CC1(C)CCc2cc(CN(Cc3cc(C(F)(F)F)cc(C(F)(F)F)c3)c3ncc(C(N)=O)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; CC1CCCc2cc(CN(Cc3cc(C(F)(F)F)cc(C(F)(F)F)c3)c3ncc(N4CCOCC4)cn3)c(N(CC3CC3)CC3CC3)nc21 ; CC1(C)CCc2cc(CN(Cc3cc(C(F)(F)F)cc(C(F)(F)F)c3)c3nn[nH]n3)c(N(CC3CC3)CC3CC3)nc21]

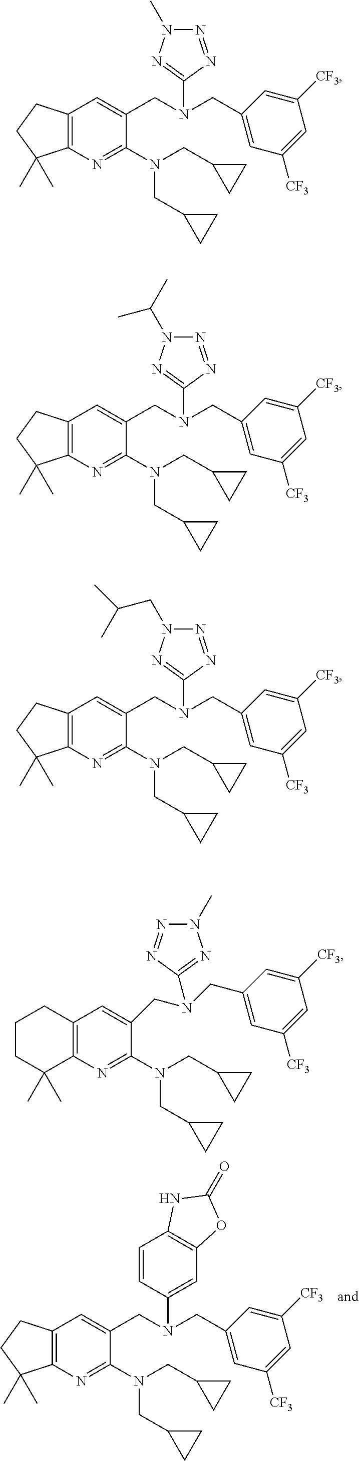

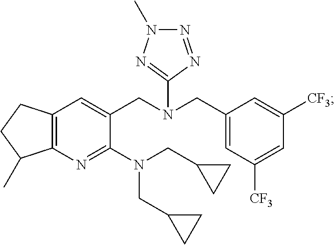 [SMILES: CC1CCc2cc(CN(Cc3cc(C(F)(F)F)cc(C(F)(F)F)c3)c3nnn(C)n3)c(N(CC3CC3)CC3CC3)nc21]

or a stereoisomer thereof or a pharmaceutically acceptable salt thereof.

In one embodiment, the application provides pharmaceutical compositions comprising one or more specific compounds of formula (III) and is enumerated as follows:

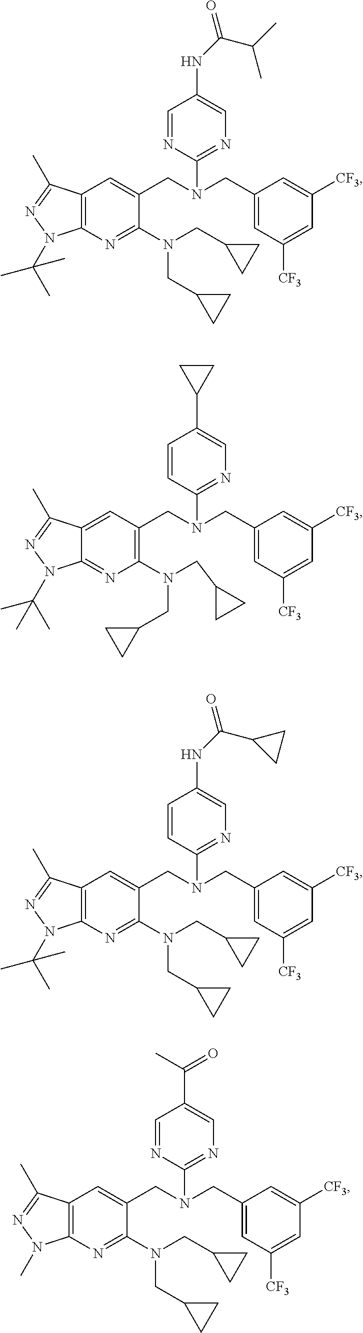

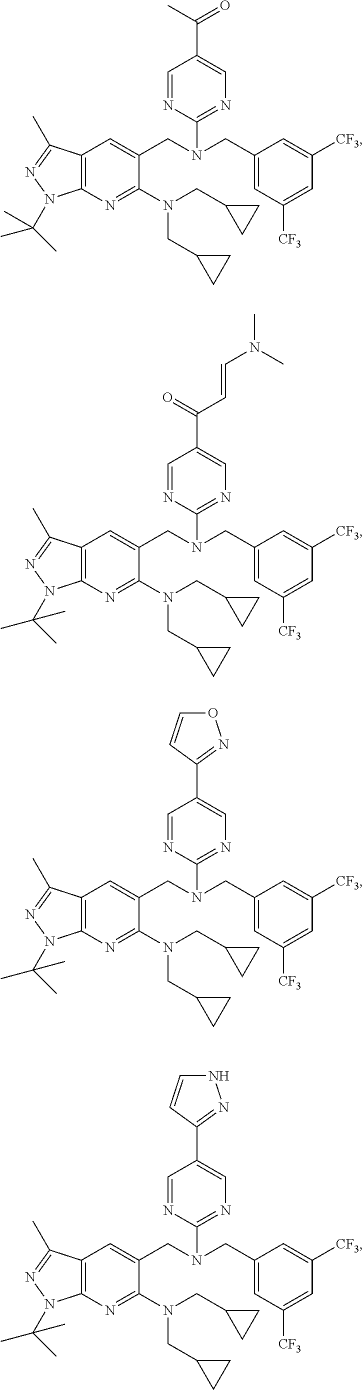

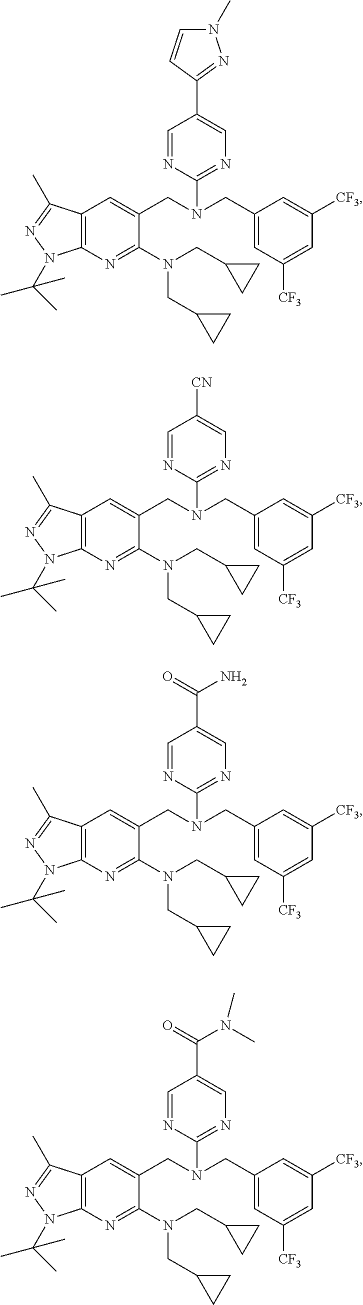

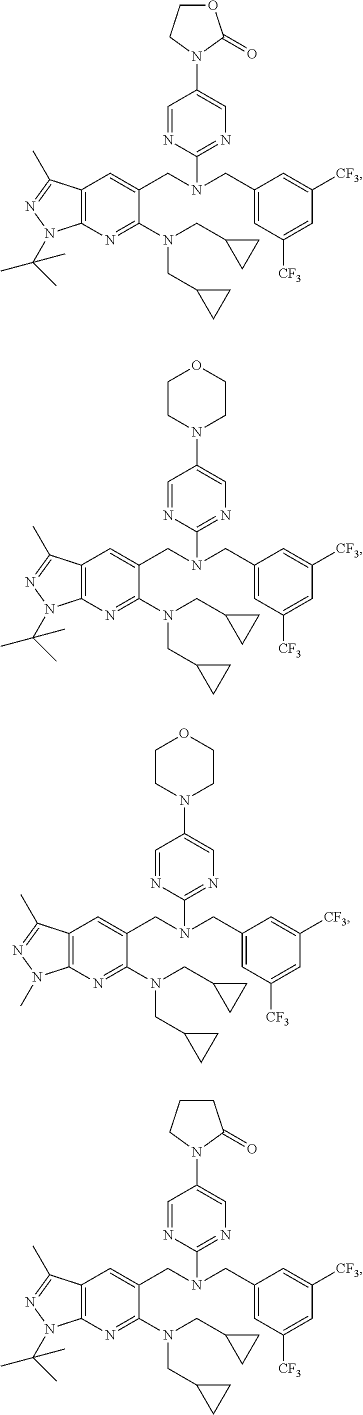

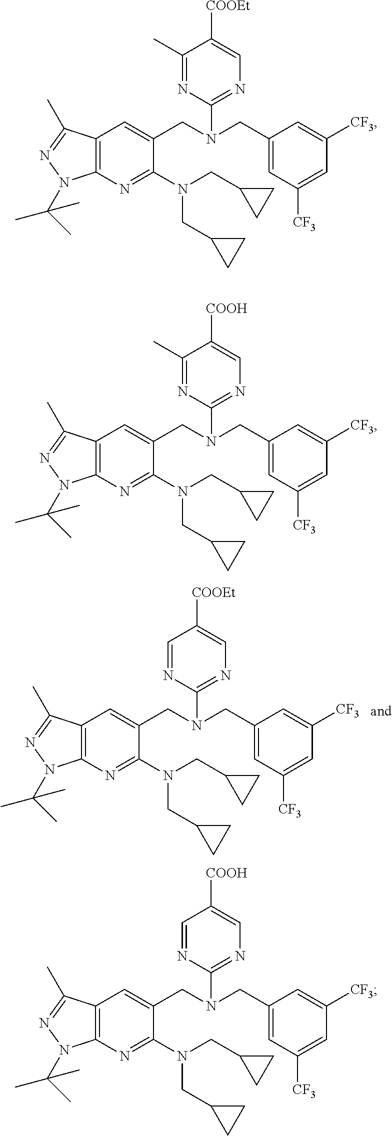

or a stereoisomer thereof or a pharmaceutically acceptable salt thereof.

In another embodiment, the application provides pharmaceutical compositions comprising one or more specific compounds of formulae (I), (Ia′), (II) or (III) and is enumerated as follows:

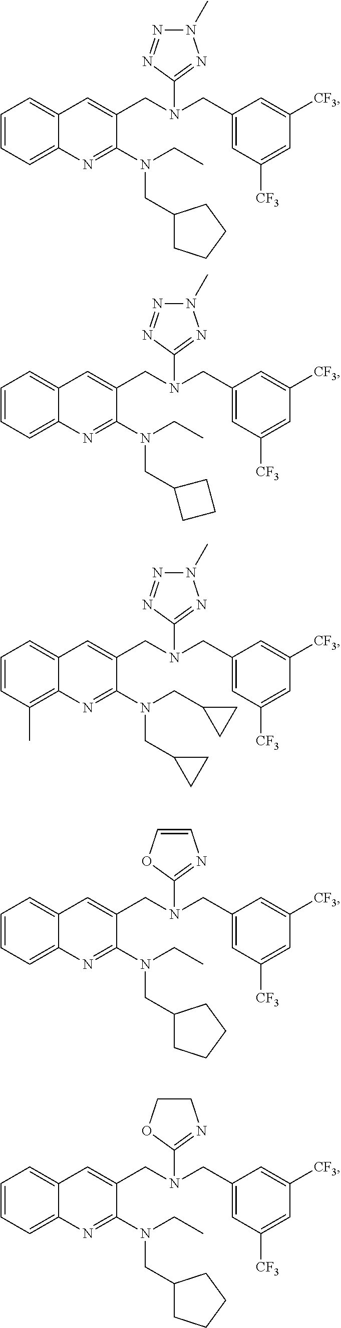

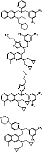

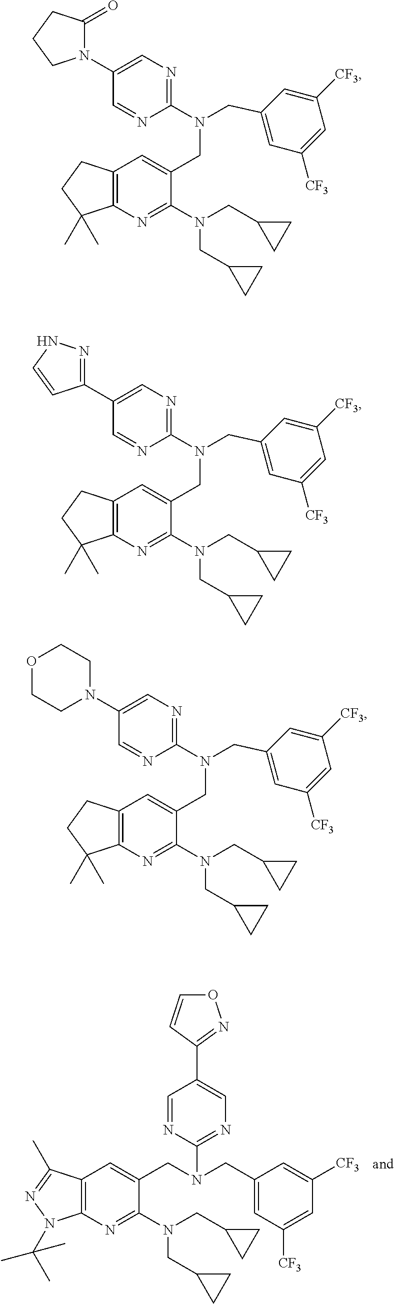 [SMILES: CC1(C)CCc2cc(CN(Cc3cc(C(F)(F)F)cc(C(F)(F)F)c3)c3ncc(N4CCCC4=O)cn3)c(N(CC3CC3)CC3CC3)nc21 ; CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; Cc1nn(C(C)(C)C)c2nc(N(CC3CC3)CC3CC3)c(CN(Cc3cc(C(F)(F)F)cc(C(F)(F)F)c3)c3ncc(-c4ccon4)cn3)cc12]

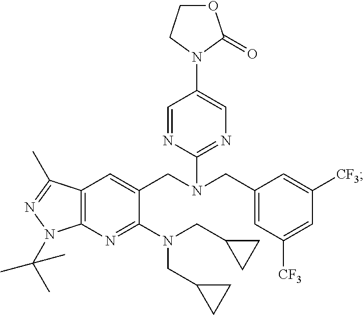 [SMILES: Cc1nn(C(C)(C)C)c2nc(N(CC3CC3)CC3CC3)c(CN(Cc3cc(C(F)(F)F)cc(C(F)(F)F)c3)c3ncc(N4CCOC4=O)cn3)cc12]

or a stereoisomer thereof or a pharmaceutically acceptable salt thereof.

In another aspect, the present application provides a composition in which the CETP inhibitor of formula (I), (Ia′), (II) or (III) is combined with at least one solubility improving material in a sufficient amount so that the composition provides maximum drug availability for absorption.

In another embodiment, the CETP inhibitor of formula (I), (Ia′), (II) or (III) of the present application may be combined with at least one solubility improving material, in the form of a solid amorphous dispersion or a solid solution or admixture or simple physical mixture.

In another aspect, the present application relates to a pharmaceutical composition comprising a solid amorphous dispersion of a CETP inhibitor of formula (I) or (Ia′) or (II) or (III) and a solubility improving material, wherein at least 10 wt % of said CETP inhibitor being noncrystalline, wherein said CETP inhibitor has a solubility in aqueous solution in the absence of said solubility improving material of less than 10 μg/ml, 2 μg/ml or less than 1 μg/ml at any pH of from 1 to 8.

In one embodiment of the above aspect, said solid amorphous dispersion comprises particles comprising both said CETP inhibitor of formula (I) or (Ia′) or (II) or (III) and said solubility improving material, and said solid amorphous dispersion has a glass transition temperature that is different than the glass transition temperature of the pure amorphous CETP inhibitor alone and different than the glass transition temperature of the pure solubility improving material alone.

In one embodiment of the above aspect, at least 10 wt % of said CETP inhibitor being noncrystalline.

In another embodiment, solubility of a CETP inhibitor in an aqueous solution in the absence of said solubility improving material of less than 10 μg/ml at any pH of from 1 to 8.

In another embodiment, solubility of a CETP inhibitor in an aqueous solution in the absence of said solubility improving material of less than 2 μg/ml at any pH of from 1 to 8.

In another embodiment, solubility of a CETP inhibitor in an aqueous solution in the absence of said solubility improving material of less than 1 μg/ml at any pH of from 1 to 8.

In another embodiment, the composition is in the form of solid amorphous dispersion.

In another embodiment, said solid amorphous dispersion has a glass transition temperature that is different than the glass transition temperature of the pure amorphous CETP inhibitor alone and different than the glass transition temperature of the pure solubility improving material alone.

In one embodiment, CETP inhibitor is selected from a compound of formula (I), which is as defined above.

In one embodiment, CETP inhibitor is selected from a compound of formula (Ia′), which is as defined above.

In one embodiment, CETP inhibitor is selected from a compound of formula (II), which is as defined above.

In one embodiment, CETP inhibitor is selected from a compound of formula (III), which is as defined above.

In another aspect, the present application provides a method of administering a pharmaceutical composition to a patient in need, wherein said composition comprising:
a) a CETP inhibitor having formula (I) or (Ia′) or (II) or (III),
b) at least one solubility improving material,
c) optionally one or more wetting agents, and
d) at least one pharmaceutically acceptable excipient.

In another aspect, the present application relates to a pharmaceutical composition comprising a dispersion of a CETP inhibitor and a solubility improving material, wherein the dispersion is sprayed on to an inert carrier in a liquid state to form a solid amorphous dispersion, wherein at least 10 wt % of said CETP inhibitor being noncrystalline, wherein said CETP inhibitor has a solubility in aqueous solution in the absence of said solubility improving material of less than 10 μg/ml, less than 2 μg/ml or less than 1 μg/ml at any pH of from 1 to 8. In one embodiment of the above aspect, said solid amorphous dispersion comprises particles comprising both said CETP inhibitor and said solubility improving material, and said solid amorphous dispersion has a glass transition temperature that is different than the glass transition temperature of the pure amorphous CETP inhibitor alone and different than the glass transition temperature of the pure solubility improving material alone.

In another embodiment, the compositions of the present application are useful in treating or preventing diseases that can be treated or prevented with CETP inhibitors, including atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorders, angina, ischemia, cardiac ischemia, stroke, myocardial infarction, reperfusion injury, angioplastic restenosis, hypertension, vascular complications of diabetes, obesity and endotoxemia. The compositions may also be useful in preventing or delaying the recurrence of certain diseases or adverse events, such as myocardial infarction, ischemia, cardiac ischemia, and stroke.

In another embodiment, the solubility improving material may typically comprise from about 5% to about 80%, from about 10% to about 75%, from about 15% to about 70% weight of the composition.

In another aspect, there is provided a process for preparing a pharmaceutical composition comprising:
a) a CETP inhibitor having formula (I) or (Ia′) or (II) or (III),
b) at least one solubility improving material,
c) optionally one or more wetting agents, and
d) at least one pharmaceutically acceptable excipient.

In another aspect, the present application relates to a pharmaceutical composition comprising:
a) a solid amorphous dispersion of CETP inhibitor having formula (I) or (Ia′) or (II) or (III) and at least one solubility improving material,
b) optionally one or more wetting agents, and
c) at least one pharmaceutically acceptable excipient.

In another aspect, there is provided a process for preparing a pharmaceutical composition comprising:
a) dissolving a CETP inhibitor having formula (I) or (Ia′) or (II) or (III) and at least one solubility improving material in one or more solvents,
b) optionally adding one or more wetting agents to the mixture of step a,
c) spray-drying the mixture of step b, to remove the solvent and to form a solid amorphous dispersion,
d) collecting the spray-dried solid amorphous dispersion powder, and
e) combining the solid amorphous dispersion powder of step d, with at least one pharmaceutically acceptable excipient to form desired dosage form.

In one embodiment of the above aspect, wherein a CETP inhibitor is selected from compound of formula (I), which is defined as earlier.

In another embodiment of the above aspect, wherein a CETP inhibitor is selected from compound of formula (Ia′), which is as defined earlier.

In another embodiment of the above aspect, wherein a CETP inhibitor is selected from compound of formula (II), which is as defined earlier.

In another embodiment of the above aspect, wherein a CETP inhibitor is selected from compound of formula (III), which is as defined earlier.

In another aspect, the solid amorphous dispersion containing CETP inhibitors and solubility improving material may be prepared by spray-coating processes, which consists of dissolution of the CETP inhibitor and at least one solubility improving material in a common solvent and spraying the mixture over inert carrier to form solid amorphous dispersion layer.

In another aspect, there is provided a process for preparing a pharmaceutical composition comprising:
a) dissolving a CETP inhibitor and at least one solubility improving material in one or more solvents,
b) optionally adding one or more wetting agents to the mixture of step a,
c) spraying the mixture of step b over inert carrier,
d) collecting the solid amorphous dispersion layered carrier, and
e) optionally combining the solid amorphous dispersion layered carrier of step d, with at least one pharmaceutically acceptable excipient to form desired dosage form.

In one embodiment of the above aspect, wherein a CETP inhibitor is selected from compound of formula (I), which is defined as earlier.

In another embodiment of the above aspect, wherein a CETP inhibitor is selected from compound of formula (Ia′), which is as defined earlier.

In another embodiment of the above aspect, wherein a CETP inhibitor is selected from compound of formula (II), which is as defined earlier.

In another embodiment of the above aspect, wherein a CETP inhibitor is selected from compound of formula (III), which is as defined earlier.

In another aspect, a pharmaceutical composition comprises a solid amorphous dispersion of a CETP inhibitor and a solubility improving material, which composition providing a maximum concentration of the CETP inhibitor in an use environment that is at least about 10-fold the maximum concentration provided by a control composition comprising an equivalent amount of the CETP inhibitor and free from the solubility improving material. As used herein, an “use environment” can be either the in vivo environment of the GI tract of a human, or the in vitro environment of a test solution, such as phosphate buffered saline (PBS) or fasted simulated gastric fluid or fasted simulated intestinal fluid or simplified simulated intestinal fluid.

It has now been found that the formulations thus formed exhibit dramatic enhancements in aqueous concentration and bioavailability when formulated using the compounds as described herein.

In one aspect, the present application provides a composition comprising a CETP inhibitor of formula (I), (Ia′), (II) or (III) and at least one solubility improving material, wherein said composition releases not more than 50% at a period of 30 minutes in 900 ml of simplified simulated intestinal fluid having a pH of 6.5, when tested in a USP Type 2 apparatus at 25 rpm and 37° C.

In another aspect, the present application provides a composition comprising a CETP inhibitor of formula (I), (Ia′), (II) or (III) and at least one solubility improving material, wherein said composition releases not more than 75% at a period of 60 minutes in 900 ml of simplified simulated intestinal fluid having a pH of 6.5, when tested in a USP Type 2 apparatus at 25 rpm and 37° C.

In yet another aspect, the present application provides a composition comprising a CETP inhibitor of formula (I), (Ia′), (II) or (III) and at least one solubility improving material, wherein said composition releases not less than 90% at a period of 360 minutes in 900 ml of simplified simulated intestinal fluid having a pH of 6.5, when tested in a USP Type 2 apparatus at 25 rpm and 37° C.

In one embodiment, the present application provides a composition comprising a CETP inhibitor of formula (I), (Ia′), (II) or (III) and at least one solubility improving material, wherein said composition when administered to a mammal provides the area under the curve (AUC₀₋₄₈) profile in fed to fast state in a ratio of about 1 to 3.

In another embodiment, the present application provides a composition comprising a CETP inhibitor of formula (I), (Ia′), (II) or (III) and at least one solubility improving material, wherein said composition when administered to a mammal provides the maximum plasma profile (Cₘₐₓ) in fed to fast state in a ratio of about 1 to 3.

The term “mammal” herein means dog, including any breeds of dogs (that includes either male or female).

The term “C” herein means the concentration of drug in blood plasma, or serum, of a subject calculated or estimated from a concentration/time curve, and is expressed in units of μM. For convenience, this concentration may be referred to herein as “drug plasma concentration”, “plasma drug concentration” or “plasma concentration”.

The term “Cmax” herein means the maximum observed blood serum concentration or the maximum blood serum concentration calculated or estimated from a concentration/time curve, and is expressed in units of μM.

The term “AUC₀₋₄₈” as used herein, means area under the plasma concentration-time curve, as calculated by the trapezoidal rule over a complete 48-hour interval.

### Solubility Improving Material:

The composition includes at least one solubility improving material. The term “solubility improving material” refers to any material present in a sufficient amount so that composition provides maximum drug availability for absorption. The maximum drug availability in absorption site, i.e. gastrointestinal (GI) tract in turn provides improved bioavailability relative to a control consisting of an equivalent amount of CETP inhibitor, without any solubility improving material.

Solubility improving material suitable for use in the various aspects of the present application should be pharmaceutically acceptable, and should have at least some solubility in aqueous solution at physiologically relevant pHs (e.g. 1-8). Almost any neutral or ionizable material that has an aqueous-solubility of at least 0.1 mg/mL over at least a portion of the pH range of 1-8 may be suitable.

The solubility improving material may be “amphiphilic” in nature, meaning having both hydrophobic and hydrophilic portions. Amphiphilic nature of polymers allows insoluble drug molecules such as CETP inhibitors to interact with the hydrophobic regions of the polymer, whereas the hydrophilic regions allow these structures to remain as stable colloids in aqueous solution, thereby maintain the drug in solubilized state in GI lumen over extended period and promote better absorption.

Solubility improving materials that may be used in the present application comprises non-ionizable (neutral) non-cellulosic polymers. Suitable examples include, but are not limited to, vinyl polymers and copolymers having substituents that are hydroxy, alkyl, acyloxy, and cyclic amides. These include polyvinyl alcohols that have at least a portion of their repeat units in the unhydrolyzed (vinyl acetate) form (e.g. polyvinyl alcohol-polyvinyl acetate copolymers); polyvinyl pyrrolidinone; polyethylene polyvinyl alcohol copolymers; and polyvinylpyrrolidinone-polyvinyl acetate copolymers. A non-cellulosic nonionic polymer also comprises polyvinylpyrrolidinone and polyvinylpyrrolidinone copolymers, such as polyvinylpyrrolidinone-polyvinyl acetate copolymers, available as Kollidon polymers and copolymers. Commercially available as KOLLIDON®VA64 (copovidone).

In one embodiment solubility improving materials may include ionizable non-cellulosic polymers. Suitable examples include, but are not limited to, carboxylic acid functionalized vinyl polymers, such as carboxylic acid functionalized polymethacrylates and carboxylic acid functionalized polyacrylates, for example, EUDRAGITS® copolymers; amine-functionalized polyacrylates and polymethacrylates; proteins; and carboxylic acid functionalized starches such as starch glycolate.

Solubility improving materials may also include non-cellulosic polymers that are amphiphilic, which are copolymers of a relatively hydrophilic and a relatively hydrophobic monomer. Examples include the acrylate and methacrylate copolymers (EUDRAGITS®) mentioned previously. Another example of amphiphilic polymers are block copolymers of ethylene oxide (or glycol) and propylene oxide (or glycol), where the poly(propylene glycol) oligomer units are relatively hydrophobic and the poly(ethylene glycol) units are relatively hydrophilic commercially sold under the tradename POLOXAMER®, and polyethylene oxide (PEO) sold under the tradename POLYOX™.

In another embodiment, such polymers may be comprised of ionizable and neutral (or non-ionizable) cellulosic polymers with at least one ester- and/or ether-linked substituent in which the polymer has a degree of substitution of at least 0.05 for each of the polymeric unit. It should be noted that the nomenclature as used herein, ether-linked substituents are recited prior to “cellulose” as the moiety attached to the ether group; for example, “ethyl cellulose” is a derivative of cellulose in which some of the hydroxyl groups on the repeating glucose units of the cellulose are converted into ethyl ether groups. Analogously, ester-linked substituents are recited after “cellulose” as the carboxylate; for example, “cellulose phthalate” has one carboxylic acid group of the phthalate moiety is reacted with one free hydroxy group of the glucose repeat unit of cellulose and the other carboxylic acid is unreacted. Similarly, “cellulose acetate phthalate” (CAP) refers to any of the family of cellulosic polymers that have acetate and phthalate groups attached via ester linkages to several of the hydroxyl groups of the glucose repeat units of the cellulose. Further cellulosic polymer family types may have additional substituents which are present relatively in small amounts such that they that do not substantially alter the performance of the resulting cellulosic polymer.

Amphiphilic cellulosics comprise polymers in which the parent cellulosic polymer has been substituted at any or all of the 3 hydroxyl groups present on each saccharide repeat unit (i.e., for example glucose repeat units) with at least one relatively hydrophobic substituent. Hydrophobic substituents may be essentially any substituent that, if substituted to a high enough level or degree of substitution, can render the cellulosic polymer essentially aqueous insoluble.

Examples of hydrophobic substituents include ether-linked alkyl groups such as methyl, ethyl, propyl, butyl, etc.; or ester-linked alkyl groups such as acetate, propionate, butyrate, etc.; and ether- and/or ester-linked aryl groups such as phenyl, benzoate, or phenylate. Hydrophilic regions of the polymer can be either those portions that are relatively unsubstituted, since the unsubstituted hydroxyls are themselves relatively hydrophilic, or those regions that are substituted with hydrophilic substituents. Hydrophilic substituents include ether- or ester-linked nonionizable groups such as the hydroxy alkyl substituents hydroxyethyl, hydroxypropyl, and the alkyl ether groups such as ethoxyethoxy or methoxyethoxy. Particularly preferred hydrophilic substituents are those that are ether- or ester-linked ionizable groups such as carboxylic acids, thiocarboxylic acids, substituted phenoxy groups, amines, phosphates or sulfonates.

In one embodiment cellulosic polymers comprise neutral polymers, which mean polymers are substantially non-ionizable in aqueous solution. Such polymers contain non-ionizable substituents, which may be either ether-linked or ester-linked. Typical ether-linked non-ionizable substituents include: alkyl groups, such as methyl, ethyl, propyl, butyl, etc.; hydroxy alkyl groups such as hydroxymethyl, hydroxyethyl, hydroxypropyl, etc.; and aryl groups such as phenyl. Typical ester-linked non-ionizable substituents include: alkyl groups, such as acetate, propionate, butyrate, etc.; and aryl groups such as phenylate. However, when aryl groups are included, the polymer may need to include a sufficient amount of a hydrophilic substituent so that the polymer has at least some water solubility at any physiologically relevant pH of from 1 to 8.

Suitable examples of non-ionizable cellulosic polymers include, but are not limited to, hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

In one embodiment neutral cellulosic polymers are amphiphilic in nature. Suitable examples of polymers include hydroxypropyl methyl cellulose and hydroxypropyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer.

A typical class of cellulosic polymers comprises polymers that are at least partially ionizable at physiologically relevant pH and include at least one ionizable substituent, which may be either ether-linked or ester-linked. Ideal ether-linked ionizable substituents include, carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, and the various isomers of picolinic acid such as ethoxypicolinic acid, etc.; thiocarboxylic acids, such as thioacetic acid; substituted phenoxy groups, such as hydroxyphenoxy, etc.; amines, such as aminoethoxy, diethylaminoethoxy, trimethylaminoethoxy, etc.; phosphates, such as phosphate ethoxy; and sulfonates, such as sulfonate ethoxy. Typical ester linked ionizable substituents include: carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as amino salicylic acid; amines, such as natural or synthetic amino acids, such as alanine or phenylalanine; phosphates, such as acetyl phosphate; and sulfonates, such as acetyl sulfonate. For aromatic-substituted polymers to also have the requisite aqueous solubility, it is also desirable that sufficient hydrophilic groups such as hydroxypropyl or carboxylic acid functional groups be attached to the polymer to render the polymer aqueous soluble at least at pH values where any ionizable groups are ionized. In some cases, the aromatic substituent may itself be ionizable, such as phthalate or trimellitate substituents.

Suitable examples of cellulosic polymers that are at least partially ionized at physiologically relevant pHs include, but are not limited to, hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, ethyl carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Cellulosic polymers that are amphiphilic in nature, having hydrophilic and hydrophobic regions include polymers such as cellulose acetate phthalate and cellulose acetate trimellitate where the cellulosic repeat units that have one or more acetate substituents are hydrophobic relative to those that have no acetate substituents or have one or more ionized phthalate or trimellitate substituents.

Most popular subset of cellulosic ionizable polymers are those that posses both a carboxylic acid functional aromatic substituent and an alkylate substituent and thus are amphiphilic. Suitable examples of such cellulosic polymers include, but are not limited to, cellulose acetate phthalate, methyl cellulose acetate phthalate, ethyl cellulose acetate phthalate, hydroxypropyl cellulose acetate phthalate, hydroxypropyl methyl cellulose phthalate, hydroxypropyl methyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Another subset of cellulosic ionizable polymers may include non-aromatic carboxylate substituent. Suitable examples of polymers may include, but are not limited to, hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, and hydroxyethyl cellulose acetate succinate.

In another embodiment polymers may consists of neutralized acidic polymers. By “neutralized acidic polymer” is meant any acidic polymer for which a significant fraction of the “acidic moieties” or “acidic substituents” have been “neutralized”; that is, exist in their deprotonated form. “Neutralized acidic cellulosic polymers” should be construed accordingly, that is, any cellulosic “acidic polymer” for which a significant fraction of the “acidic moieties” or “acidic substituents” have been “neutralized.” By “acidic polymer” is meant any polymer that possesses a significant number of acidic moieties. In general, a significant number of acidic moieties would be greater than or equal to about 0.1 milliequivalents of acidic moieties per gram of polymer. “Acidic moieties” include any functional groups that are sufficiently acidic that, in contact with or dissolved in water, can at least partially donate a hydrogen cation to water and thus increase the hydrogen-ion concentration. This definition includes any functional group or “substituent,” as it is termed when the functional group is covalently attached to a polymer that has a pKa of less than about 10. Suitable classes of functional groups that are included in the above description include carboxylic acids, thiocarboxylic acids, phosphates, phenolic groups, and sulfonates. Such functional groups may make up the primary structure of the polymer such as for polyacrylic acid, but more generally are covalently attached to the backbone of the parent polymer and thus are termed “substituents.”

When specific polymers that are suitable for use in the compositions of the present invention are blended, the blends of such polymers may also be suitable. Thus the term “solubility improving material” is intended to include blends of polymers in addition to a single species of polymer.

In one embodiment, the solubility improving materials may include a blend of ionizable non-cellulosic and ionizable cellulosic polymers, ionizable non-cellulosic and non-ionizable cellulosic polymers, ionizable non-cellulosic and non-ionizable non-cellulosic polymers, or any combinations thereof.

### Wetting Agents

The composition of the present application optionally includes one or more wetting agents. It is contemplated that the wetting agent generally increases the rate of dissolution by facilitating wetting, thereby increasing the maximum concentration of the dissolved drug. The wetting agents can also be employed in the preparation of dispersion(s) containing one or more of the CETP inhibitors as described herein. It has also been contemplated that the wetting agents generally stabilize the amorphous dispersions by inhibiting crystallization or precipitation of the drug by interacting with the dissolved drug by such mechanisms as complexation, formation of inclusion complexes, formation of micelles, and adsorption to the surface of the solid drug, among various other possible mechanisms.

Wetting agents may be of cationic, anionic, and nonionic in nature. Suitable examples of wetting agents include, but are not limited to, fatty acids and alkyl sulfonates; cationic wetting agents such as benzalkonium chloride (HYAMINE 1622, available from Lonza, Inc., Fairlawn, N.J.); anionic wetting agents, such as dioctyl sodium sulfosuccinate (Docusate Sodium) and sodium lauryl sulfate (sodium dodecyl sulfate); sorbitan fatty acid esters (SPAN series of surfactants); Vitamin E TPGS; polyoxyethylene sorbitan fatty acid esters (TWEEN series of surfactants, available from ICI Americas Inc., Wilmington, Del.); polyoxyethylene castor oils and hydrogenated castor oils such as CREMOPHOR RH-40 and CREMOPHOR EL; LIPOSORB P-20, available from Lipochem Inc., Patterson N.J.; CAPMUL POE-0, available from Abitec Corp., Janesville, Wis.), and natural surfactants such as sodium taurocholic acid, 1-palmitoyl-2-oleoyl-sn-glycero-3-phosphocholine, lecithin, and other phospholipids and mono- and diglycerides, polyoxyethylene fatty acid glycerides, stearyl alcohol, cetostearyl alcohol, cholesterol, polyoxyethylene ricin oil, polyethylene glycol glycerides (e.g., GELUCIRE®) poloxamers (e.g., PLURONICS F68® and F1 08®, which are block copolymers of ethylene oxide and propylene oxide) and mixtures thereof.

In one embodiment, the wetting agent may typically comprise up to about 15%, up to about 12.5%, up to about 10%, up to about 7.5% weight of the composition.

### Pharmaceutically Acceptable Excipients:

The composition of the present application may contain suitable amounts of pharmaceutically acceptable excipients that would be necessary for preparing appropriate dosage forms. Examples of pharmaceutically acceptable excipients that can be used in the composition of the present invention include, but not limited to, one or more diluents, binders, disintegrants, lubricants/glidants, buffers, coloring agents, flavoring agents or combinations thereof.

Examples of fillers or diluents include, but not limited to, corn starch, lactose, white sugar, sucrose, sugar compressible, sugar confectioners, glucose, sorbitol, calcium carbonate, calcium dihydrogen phosphate dihydrate, calcium phosphate-dibasic, calcium phosphate-tribasic, calcium sulfate, microcrystalline celluloses (MCC, e.g. CEOLUS™ UF/KG/PH), silicified MCC (e.g., PROSOLV™ HD 90, PROSOLV™ SMCC 90), cellulose powdered, dextrates, dextrins, dextrose, fructose, kaolin, lactitol, mannitol, starch, starch pregelatinized and combinations comprising one or more of the foregoing materials.

Examples of binders include, but not limited to, povidones, various starches known in the art, including corn starch, pregelatinized starch, microcrystalline celluloses (MCC, e.g. CEOLUS™ UF/KG/PH), silicified MCC (e.g., PROSOLV™ HD 90, PROSOLV™ SMCC 90), microfine celluloses, lactose, calcium carbonate, calcium sulfate, sugar, mannitol, sorbitol, dextrates, dextrin, maltodextrin, dextrose, dibasic calcium phosphate dihydrate, tribasic calcium phosphate, magnesium carbonate, magnesium oxide, stearic acid, gums, hydroxypropyl methylcellulose or hypromelloses (e.g., KLUCEL™EF, METHOCEL™ E5 premium) and other pharmaceutically acceptable substances with cohesive properties.

Examples of disintegrants include, but not limited to, cross-linked polyvinyl pyrrolidone, corn starch, potato starch, maize starch and modified starches, agar-agar, calcium carbonate, sodium carbonate, alginic acids, cross-carmellose sodium, sodium starch glycolate, microcrystalline cellulose and mixtures thereof.

Examples of lubricants and glidants that can be used in the present invention include, but are not limited to, colloidal silicon dioxide, such as AEROSIL® 200, talc, stearic acid, magnesium stearate, calcium stearate, solid polyethylene glycols, sodium stearyl fumarate, silica gel and mixtures thereof and other substances with lubricating or gliding properties.

Examples of buffers that can be used include, but not limited to, phosphate, acetate, citrate, succinate and histidine buffers.

The coloring agents and flavoring agents can also be used and may be selected from any FDA approved colors and flavors for oral use.

### Dosage Forms and Process for Preparation:

The composition of the present application may be prepared as oral dosage forms such as tablets, pills, capsules, powders, powders for suspension, suspensions, granules and/or microgranules.

In one aspect of the present application, the ratio of CETP inhibitor and solubility improving material relative to the other excipients of the composition may be in the range of 1:0.1 to 1:10, respectively.

In one embodiment, the composition comprising CETP inhibitors of the present application may be processed with at least one solubility improving material, in the form of solid amorphous dispersion or solid solution or admixture or simple physical mixture.

Solid amorphous dispersions of CETP inhibitors of the present application may be prepared according to any known process which results in amorphous state. The amorphous state of the CETP inhibitors in the composition may be at least 10%, at least 20%, at least 40%, or at least 60%. The CETP inhibitors present in the amorphous dispersions may be substantially amorphous and may be substantially homogeneously distributed throughout the solubility improving material. The relative amounts of crystalline and CETP inhibitors of the present invention can be determined by several analytical methods, including differential scanning calorimetry (DSC) and x-ray powder diffraction (XRPD).

The processes for preparing solid amorphous dispersions include, milling and extrusion; melt processes, such as high temperature fusion, hot melt extrusion, fusion process, and melt congealing processes; and solvent processes, including non-solvent precipitation processes, spray coating, and spray-drying. The dispersions of the present application may be made by any of these processes, the CETP inhibitors in the dispersions generally have maximum bioavailability and stability.

In general, as the degree of homogeneity of the dispersion increases, the availability of the CETP inhibitors for absorption increases thereby increasing the relative bioavailability as well. The dispersions of the invention may have single glass transition temperature, indicating high degree of homogeneity between the drug and the solubility improving material.

In one embodiment, the amount of CETP inhibitor and the solubility improving material present in the dispersions of the present application may be in a ratio of about 1:0.1 to about 1:20 The CETP inhibitor: solubility improving material ratio that yields optimum results varies from compound to compound and is best determined by in vitro dissolution tests and/or in vivo bioavailability tests.

The term “solid amorphous dispersion” refers to that composition of CETP inhibitor (i.e., the drug) and solubility improving material, which is completely homogeneous and in which the CETP inhibitor is substantially amorphous. The amorphous drug may exist in the drug/solubility improving material dispersion as a solid solution of drug homogeneously distributed throughout the dispersion, or a portion of the drug may exist in relatively drug-rich domains. The solid amorphous dispersion is substantially homogeneous so that the amorphous drug is dispersed as homogeneously as possible throughout the dispersion.

The solid amorphous dispersion may have some drug-rich domains, and the dispersion may have a single glass-transition temperature (T_{g}). This contrasts with a simple physical mixture of amorphous drug particles and solubility improving material. Such physical mixtures generally display two distinct T_{g} values, one that of the drug and the other one of the solubility improving material. When the matrix is not amorphous or does not have a T_{g}, the T_{g} of the simple physical mixture generally has the same T_{g} of pure amorphous drug particles alone. Dispersions of the present application that are substantially homogeneous generally are more physically and chemically stable.

The solid amorphous dispersion containing CETP inhibitors of the present application and solubility improving material may be prepared by “solvent processing” which consists of dissolution of the CETP inhibitor and at least one solubility improving materials in a common solvent. “Common solvent” as used herein means that a single solvent, which can be comprised of a mixture of compounds (i.e., solvents), will simultaneously dissolve the drug and the solubility improving material(s). After both the CETP inhibitor and the solubility improving material have been dissolved, the solvent is rapidly removed by evaporation or by mixing with a non-solvent. Typical processes that are known in the art which can be employed herein include without any limitation spray-drying, spray-coating (pan-coating, fluidized bed coating, etc.), and precipitation by rapid mixing of the polymer and drug solution with CO₂, water, or some other non-solvent. Removal of the solvent results in a solid amorphous dispersion which is substantially homogeneous.

The solvent may be removed through the process of spray-drying. The term spray-drying as used herein shall have the conventional meaning and broadly refers to processes involving breaking up of liquid mixtures into small droplets (atomization) and rapidly removing solvent from the mixture in a container (spray-drying apparatus) where there is a strong driving force for evaporation of solvent from the droplets. The strong driving force for solvent evaporation is generally provided by maintaining the partial pressure of solvent in the spray-drying apparatus well below the vapor pressure of the solvent at the temperature of the drying droplets. In addition, at least a portion of the heat required for evaporation of solvent may be provided by heating the spray solution.

Solvents suitable for spray-drying can be any organic compound in which the CETP inhibitor and one solubility improving material are mutually soluble. The solvent should be volatile with a boiling point of 150° C. or less. Examples of solvents include, but are not limited to, alcohols such as methanol, ethanol, n-propanol, iso-propanol, and butanol; ketones such as acetone, methyl ethyl ketone and methyl iso-butyl ketone; esters such as ethyl acetate and propyl acetate; and various other solvents such as acetonitrile, methylene chloride, toluene, 1,1,1-trichloroethane and mixtures in any combinations thereof. Other solvents such as dimethyl acetamide or dimethylsulfoxide can also be used.

In one embodiment, the process may yield single layered, double layered or multi-layered dispersions over inert carrier, in order to have increased concentration of drug at the site of absorption, i.e., gastrointestinal tract. The drug dispersion layered carrier may be further combined with other pharmaceutically acceptable excipients to form desired dosage form. The inert carriers on which the drug dispersion may be layered include crystals or sugars or inorganic salts such as crystal lactose, crystalline cellulose and crystal sodium chloride, and spherical granulation products (such as the spherical granulation product of crystalline cellulose (trade name: AVICEL® SP), the spherical granulation product of crystalline cellulose and lactose (trade name: NONPAREIL® NP-5 and NP-7), the spherical granulation product of refined sucrose (trade name: NONPAREIL®-103), and the spherical granulation product of lactose and alpha-converted starch. The inert carriers may be prepared by blending microcrystalline cellulose, silified microcrystalline cellulose and hydroxypropylcellulose and then the blend was further granulated using hydroxypropylcellulose solution. The resultant granules were dried and sieved for further use.

In another aspect, the solid dispersion containing CETP inhibitor and solubility improving material may be formed by a thermal process, such as an extrusion process, a fusion process, or a melt-congeal process. In such cases, a matrix is selected such that it is suitable for use in the thermal process. Generally, it is desirable to keep the processing temperature as low as possible to avoid thermal degradation of the drug. It is desired that the matrix as a whole become fluid at a temperature of less than about 200° C., less than about 160° C., or less than about 120° C. A matrix that becomes fluid at a higher temperature than this should only be used with drugs that are thermally stable at the required processing temperature.

Suitable examples that are suitable for use as a matrix component for thermal processes include, but are not limited to, alcohols, such as stearyl alcohol and cetyl alcohol, organic acids, such as stearic acid, citric acid, and malic acid; sugars such as glucose, xylitol, sorbitol, and maltitol; fatty acid esters such as mono-, di-, and tri-glycerides, glyceryl mono-, di-, and tri-stearates, glyceryl mono-, di-, and tri-behenates, sorbitan monostearate, saccharose monostearate, glyceryl (palmitic stearic) ester, polyoxyethylene sorbitan fatty-acid esters; waxes, such as microcrystalline wax, paraffin wax, beeswax, synthetic wax, castor wax, and carnauba wax; alkyl sulfates such as sodium lauryl sulfate; and polymers such as polyethylene glycols, polyoxyethylene glycols, polyethylene-propylene glycol copolymers, poloxamers, polyethylene oxide, polyvinyl pyrrolidinone (also referred to as polyvinyl pyrrolidone or povidone or PVP), polyvinyl alcohol, polyethylene-vinyl alcohol copolymers, polyvinyl alcohol polyvinyl acetate copolymers, carboxylic acid-functionalized polymethacrylates, amine-functionalized polymethacrylates and mixtures thereof.

The matrix may include a plasticizer as one component of the matrix to reduce processing temperature. Suitable plasticizers may include but are not limited to, mineral oils, petrolatum, lanolin alcohols, polyethylene glycol, polypropylene glycol, sorbitol, triethanol amine, benzyl benzoate, dibutyl sebacate, diethyl phthalate, glyceryl monostearate, triacetin, and triethyl citrate. Solvents or swelling agents, such as water, alcohols, ketones, and the like may also be used to reduce processing temperature and improve the processability of the composition.

Once the molten mixture is formed, it may be mixed to ensure the drug is homogeneously distributed throughout the molten mixture. Such mixing may be done using mechanical means, such as overhead mixers, magnetically driven mixers and stir bars, planetary mixers, mixing bowls, and homogenizers. Optionally, when the molten mixture is formed in a vessel, the contents of the vessel can be pumped out of the vessel and through an in-line or static mixer and then returned to the vessel. The amount of shear used to mix the molten mixture should be sufficiently high to ensure uniform distribution of the drug in the molten mixture. The molten mixture can be mixed from a few minutes to several hours, the mixing time being dependent on the viscosity of the mixture and the solubility of the drug and any optional excipients in the solubility improving material.

Another method of preparing the molten mixture is to use two vessels, melting the drug and optionally, the wetting agent in the first vessel and the solubility improving material and optionally, wetting agent in a second vessel. The two melts are then pumped through an in-line static mixer or extruder to produce the molten mixture that is then rapidly solidified.

On the other hand, the molten mixture can be generated using an extruder, such as a single-screw or twin-screw extruder, both well known in the art. In such devices, a solid, or semi-solid mixture of the composition is fed to the extruder whereby the combination of heat and shear forces within the extruder produce a uniformly mixed molten mixture, which can then be rapidly solidified to form the solid amorphous dispersion. The solid feed can be prepared using methods well known in the art for obtaining solid mixtures with high content uniformity. Alternatively, the extruder may be equipped with two or more feeders, allowing the drug, and optionally the wetting agent, to be fed to the extruder through one feeder and the solubility improving material, and optionally the wetting agent, through the other. Other excipients to reduce the processing temperature as described above may be included in the solid feed, or in the case of liquid excipients, such as water, may be injected into the extruder using methods well-known in the art.

The extruder should be designed such that it produces a molten mixture with the drug uniformly distributed throughout the composition. The various zones in the extruder should be heated to appropriate temperatures to obtain the desired extrudate temperature as well as the desired degree of mixing or shear, using procedures well known in the art.

When the drug has a high solubility in the matrix, a lower amount of mechanical energy will be required to form the dispersion. In such cases, the processing temperature may be below the melting temperature of the undispersed amorphous drug but greater than the melting point of at least a portion of the matrix materials, since the drug will dissolve into the molten matrix. When the drug has a low-solubility in the matrix, a higher amount of mechanical energy may be required to form the dispersion. Here, the processing temperature may need to be above the melting point of the drug and at least some of the matrix components. A high amount of mechanical energy may be needed to mix the molten drug with the matrix components to form a homogeneous dispersion. Typically, the lowest processing temperature and an extruder design that imparts the lowest amount of mechanical energy (e.g., shear) that produce a satisfactory dispersion is chosen in order to minimize the exposure of drug to harsh conditions.

Once the molten mixture of drug, solubility improving material, and optionally the wetting agent is formed, the mixture should be rapidly solidified to form the solid amorphous dispersion. Rapid solidification is only necessary when the drug and other materials in the molten mixture are not miscible. By “rapidly solidified” is meant that the molten mixture is solidified sufficiently fast such that substantial phase separation of the drug from the other materials does not occur. In general, this means that the mixture should be solidified in less than about 10 minutes, less than about 5 minutes, less than about 1 minute. If the mixture is not rapidly solidified, phase separation can occur, if the materials are not miscible at storage temperatures, resulting in the formation of drug-rich phases.

Solidification often takes place primarily by cooling the molten mixture to at least about 10° C. and at least about 30° C. below its melting point. As mentioned above, solidification can be additionally promoted by evaporation of all or part of one or more volatile excipients or solvents. To promote rapid cooling and evaporation of volatile excipients, the molten mixture is often formed into a high surface area shape such as a rod or fiber or droplets. For example, the molten mixture can be forced through one or more small holes to form long thin fibers or rods or may be fed to a device, such as an atomizer such as a rotating disk that breaks the molten mixture up into droplets. The droplets are then contacted with a relatively cool fluid such as air or nitrogen to promote cooling and evaporation.

The solid amorphous dispersion formed in above processes can be further processed with other pharmaceutically acceptable excipients to form desired dosage forms.

In another aspect, the present application relates to a pharmaceutical composition comprising a CETP inhibitor, at least one solubility improving material and optionally one or more wetting agents, wherein the CETP inhibitor and the solubility improving material are simply admixed.

The term “admixed” refers to those compositions of CETP inhibitor and solubility improving material which are simple physical mixtures of the type achieved by combining and physically stirring dry components together. Such physical mixtures include wet and dry granulated mixtures. As is known in the art, granulation is a process used to improve the handling and manufacturing properties of a formulation, for example by increasing particle size to improve flow. Granulation may not substantially change the physical form of the drug such as its crystalline or amorphous character.

The compositions of the present application may be prepared by dry- or wet-mixing the drug or drug mixture with the at least one solubility improving material, to form the composition. Mixing processes that can be employed include physical processing as well as wet-granulation and coating processes among various other known processes.

For example, mixing methods include convective mixing, shear mixing, or diffusive mixing. Convective mixing involves moving a relatively large mass of material from one part of a powder bed to another, by means of blades or paddles, revolving screw, or an inversion of the powder bed. Shear mixing occurs when slip planes are formed in the material to be mixed. Diffusive mixing involves an exchange of position by single particles. These mixing processes can be performed using equipment in batch or continuous mode. Tumbling mixers (e.g., twin-shell) are commonly used equipment for batch processing. Continuous mixing can be used to improve composition uniformity.

Milling may also be employed to prepare the compositions of the present application. Milling is the mechanical process of reducing the particle size of solids (comminution). The most common types of milling equipment are the rotary cutter, the hammer, the roller and fluid energy mills. Equipment choice depends on the characteristics of the ingredients in the drug form (e.g., soft, abrasive, or friable). Wet- or dry-milling techniques can be chosen for several of these processes, also depending on the characteristics of the ingredients (e.g. drug stability in solvent). The milling process may serve simultaneously as a mixing process if the feed materials are heterogeneous.

In further aspect compositions of the present application may be used to treat any condition which is subject to treatment by administering a CETP inhibitor.

One aspect of this application is directed to a method for treating atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorders, angina, ischemia, cardiac ischemia, stroke, myocardial infarction, reperfusion injury, angioplastic restenosis, hypertension, vascular complications of diabetes, obesity or endotoxemia in a patient (including a human being, either male or female) by administering to a patient in need of such treatment an atherosclerotic treating amount of a composition of the present invention.

In another aspect of this application, the pharmaceutical compositions as disclosed herein are used in the treatment of various aforementioned diseases.

The present invention is illustrated below by reference to the following examples. However, one skilled in the art will appreciate that the specific methods and results discussed are merely illustrative of the invention, and not to be construed as limiting the invention.

### EXAMPLES

In the following Examples 1-17, various compositions in accordance with the present application were prepared comprising 3-(((3,5-bis(trifluoromethyl)benzyl)(2-methyl-2H-tetrazol-5-yl)amino)methyl)-N,N-bis(cyclopropylmethyl)-8-methylquinolin-2-amine as the CETP inhibitor:

### Example 1

| Ingredients | Percent w/w |
|---|---|
| 3-(((3,5-bis(trifluoromethyl)benzyl)(2-methyl-2H- | 18.15 |
| tetrazol-5-yl)amino)methyl)-N,N- | |
| bis(cyclopropylmethyl)-8-methylquinolin-2-amine | |
| Hydroxypropyl methyl cellulose acetate succinate | 36.29 |
| (AQOAT ®LF) | |
| Polyoxyl 35 castor oil (CREMOPHOR ® EL) | 3.63 |
| Talc | 3.63 |
| Sugar spheres | 32.66 |
| Dichloromethane | q.s. |
| Methanol | q.s. |

Seal layer

| | |
|---|---|
| Polyethylene glycol 6000 | 4.34 |
| Talc | 1.30 |
| Isopropyl alcohol | q.s. |
| water | q.s. |

### Process:

1. 3-(((3,5-bis(trifluoromethyl)benzyl)(2-methyl-2H-tetrazol-5-yl)amino)methyl)-N,N-bis(cyclopropylmethyl)-8-methylquinolin-2-amine and hydroxypropyl methyl cellulose acetate succinate were mixed together in given solvent mixture to form clear solution.
2. To the solution of step 1, Polyoxyl 35 castor oil and talc were added to form a homogenous suspension.
3. The suspension of step 2 was sprayed over inert sugar spheres and dried.
4. The drug layered spheres of step 3 were coated with dispersion made from given seal layer ingredients.
5. The coated spheres of step 4 were formulated further as capsule dosage form.

### Example 2

| Ingredients | Percent w/w |
|---|---|
| 3-(((3,5-bis(trifluoromethyl)benzyl)(2-methyl- | 7.14 |
| 2H-tetrazol-5-yl)amino)methyl)-N,N- | |
| bis(cyclopropylmethyl)-8-methylquinolin-2- | |
| amine | |
| Hydroxypropyl methyl cellulose acetate | 28.57 |
| succinate (AQOAT ®MF) | |
| Polyoxyl 35 castor oil (CREMOPHOR ® EL) | 2.86 |
| Talc | 2.86 |
| Sugar spheres | 12.86 |
| Dichloromethane | q.s. |
| Methanol | q.s. |

Seal layer

| | |
|---|---|
| Polyethylene glycol 6000 | 1.66 |
| Talc | 0.51 |
| Isopropyl alcohol | q.s. |
| water | q.s. |

Extragranular ingredients

| | |
|---|---|
| Croscarmellose sodium | 10.71 |
| Colliodal silicon dioxide | 0.71 |
| Magnesium stearate | 1.07 |
| Polyvinylpyrrolidinone-polyvinyl acetate | 14.29 |
| copolymer (KOLLIDON ® VA64) | |
| Silicified microcrystalline cellulose | 9.61 |
| Polyethylene glycol 20000 | 7.14 |

### Process:

1. 3-(((3,5-bis(trifluoromethyl)benzyl)(2-methyl-2H-tetrazol-5-yl)amino)methyl)-N,N-bis(cyclopropylmethyl)-8-methylquinolin-2-amine and hydroxypropyl methyl cellulose acetate succinate were mixed together in given solvent mixture to form clear solution.
2. To the solution of step 1, Polyoxyl 35 castor oil and talc were added to form a homogenous suspension.
3. The suspension of step 2 was sprayed over inert sugar spheres and dried.
4. The drug layered spheres of step 3 were coated with dispersion made from given seal layer ingredients.
5. The coated spheres of step 4 were further blended with given extragranular ingredients.
6. The blend of step 5 was compressed into tablets using suitable tooling.

### Example 3

| Ingredients | Percent w/w |
|---|---|
| 3-(((3,5-bis(trifluoromethyl)benzyl)(2-methyl- | 21.26 |
| 2H-tetrazol-5-yl)amino)methyl)-N,N- | |
| bis(cyclopropylmethyl)-8-methylquinolin-2- | |
| amine | |
| Hydroxypropyl methyl cellulose 3 cps | 31.89 |
| Sugar spheres | 42.52 |
| Isopropyl alcohol | q.s. |
| Water | q.s. |

Seal layer

| | |
|---|---|
| Polyethylene glycol 6000 | 3.83 |
| Isopropyl alcohol | q.s. |
| water | q.s. |

Lubrication

| | |
|---|---|
| Talc | 0.50 |

### Process:

1. 3-(((3,5-bis(trifluoromethyl)benzyl)(2-methyl-2H-tetrazol-5-yl)amino)methyl)-N,N-bis(cyclopropylmethyl)-8-methylquinolin-2-amine and hydroxypropyl methyl cellulose were mixed together in given solvent to form clear solution
2. The solution of step 1 was sprayed over inert sugar spheres and dried
3. The drug layered spheres of step 2 were coated with dispersion made from given seal layer ingredients
4. The coated spheres of step 3 were lubricated with talc and filled in capsules.

### Example 4

| Ingredients | Percent w/w |
|---|---|
| 3-(((3,5-bis(trifluoromethyl)benzyl)(2-methyl-2H- | 13.33 |
| tetrazol-5-yl)amino)methyl)-N,N- | |
| bis(cyclopropylmethyl)-8-methylquinolin-2-amine | |
| Hydroxypropyl methyl cellulose acetate succinate | 26.67 |
| (AQOAT ®LF) | |
| Dichloromethane | q.s. |
| Methanol | q.s. |
| Silicified microcrystalline cellulose | 13.33 |
| Lactose monohydrate | 40 |
| Colliodal silicon dioxide | 0.80 |
| Croscarmellose sodium | 4.80 |
| Magnesium stearate | 1.07 |

### Process:

1. 3-(((3,5-bis(trifluoromethyl)benzyl)(2-methyl-2H-tetrazol-5-yl)amino)methyl)-N,N-bis(cyclopropylmethyl)-8-methylquinolin-2-amine and hydroxypropyl methyl cellulose acetate succinate were mixed together in given solvent mixture to form clear solution.
2. The solution of step 1 was spray dried in laboratory spray-drier.
3. Solid spray dried material was collected and mixed with microcrystalline cellulose, lactose monohydrate, polyethylene glycol 6000 and croscarmellose sodium.
4. Powder blend of step 3 was sieved together and blended to get uniform powder mixture.
5. Blend of step 4 was lubricated with magnesium stearate and compressed into tablets using suitable tooling.

### Example 5-7

| | Percent w/w | | |
|---|---|---|---|
| Ingredients | Example 5 | Example 6 | Example 7 |
| 3-(((3,5- | 19.23 | 15.63 | 13.16 |
| bis(trifruoromethyl)benzyl)(2- | | | |
| methyl-2H-tetrazol-5- | | | |
| yl)amino)methyl)-N,N- | | | |
| bis(cyclopropylmethyl)-8- | | | |
| methylquinolin-2-amine | | | |
| Hydroxypropyl methyl cellulose | 38.46 | 46.88 | 52.63 |
| acetate succinate (AQOAT ®LF) | | | |
| Polyoxyl 35 castor oil | 3.85 | 4.69 | 5.26 |
| (CREMOPHOR ® EL) | | | |
| Talc | 3.85 | 4.69 | 5.26 |
| Sugar spheres | 34.62 | 28.13 | 23.68 |
| Dichloromethane | q.s. | q.s. | q.s. |
| Methanol | q.s. | q.s. | q.s. |

### Process:

1. 3-(((3,5-bis(trifluoromethyl)benzyl)(2-methyl-2H-tetrazol-5-yl)amino)methyl)-N,N-bis(cyclopropylmethyl)-8-methylquinolin-2-amine and hydroxypropyl methyl cellulose acetate succinate were mixed together in given solvent mixture to form clear solution.
2. To the solution of step 1, Polyoxyl 35 castor oil and talc were added to form homogenous suspension.
3. The suspension of step 2 was sprayed over inert sugar spheres and dried.
4. The coated spheres of step 3 were formulated further as capsule dosage form.

### Example 8-11

| | Percent w/w | | | |
|---|---|---|---|---|
| | Exam- | Exam- | Exam- | Exam- |
| Ingredients | ple 8 | ple 9 | ple 10 | ple 11 |
| 3-(((3,5- | 19.23 | 15.63 | 13.16 | 17.24 |
| bis(trifruoromethyl)benzyl)(2- | | | | |
| methyl-2H-tetrazol-5- | | | | |
| yl)amino)methyl)-N,N- | | | | |
| bis(cyclopropylmethyl)-8- | | | | |
| methylquinolin-2-amine | | | | |
| Hydroxypropyl methyl | 38.46 | 46.88 | 52.63 | 51.72 |
| cellulose acetate succinate | | | | |
| (AQOAT ®MF) | | | | |
| Polyoxyl 35 castor oil | 3.85 | 4.69 | 5.26 | 5.17 |
| (CREMOPHOR ® EL) | | | | |
| Talc | 3.85 | 4.69 | 5.26 | 2.59 |
| Sugar spheres | 34.62 | 28.13 | 23.68 | 23.28 |
| Dichloromethane | q.s. | q.s. | q.s. | q.s. |
| Methanol | q.s. | q.s. | q.s. | q.s. |

### Process:

1. 3-(((3,5-bis(trifluoromethyl)benzyl)(2-methyl-2H-tetrazol-5-yl)amino)methyl)-N,N-bis(cyclopropylmethyl)-8-methylquinolin-2-amine and hydroxypropyl methyl cellulose acetate succinate were mixed together in given solvent mixture to form clear solution.
2. To the solution of step 1, Polyoxyl 35 castor oil and talc were added to form homogenous suspension.
3. The suspension of step 2 was sprayed over inert sugar spheres and dried.
4. The coated spheres of step 3 were formulated further as capsule dosage form.

### Example 12

| Ingredients | Percent w/w |
|---|---|
| 3-(((3,5-bis(trifluoromethyl)benzyl)(2-methyl-2H- | 16.13 |
| tetrazol-5-yl)amino)methyl)-N,N- | |
| bis(cyclopropylmethyl)-8-methylquinolin-2-amine | |
| Hydroxypropyl methyl cellulose acetate succinate | 64.35 |
| (AQOAT ®MF) | |
| Triethyl citrate | 19.35 |

### Process:

1. Hydroxypropyl methyl cellulose acetate succinate and triethyl citrate were mixed together for 30 minutes.
2. To the mixture of step 1, 3-(((3,5-bis(trifluoromethyl)benzyl)(2-methyl-2H-tetrazol-5-yl)amino)methyl)-N,N-bis(cyclopropylmethyl)-8-methylquinolin-2-amine was added and blended well.
3. The pre-blend of step 2 was fed to melt extruder, wherein the extruder was set at a temperature of 95° C. and the screw speed was set at 1000 RPM.
4. The extrudate exited the extruder was cooled in air to solidify the extrudates.
5. The extrudates of step 4 was milled formulated further as capsule dosage form.

### Example 13-14

| | Percent w/w | |
|---|---|---|
| Ingredients | Example 13 | Example 14 |
| 3-(((3,5-bis(trifluoromethyl)benzyl)(2- | 9.26 | 7.2 |
| methyl-2H-tetrazol-5- | | |
| yl)amino)methyl)-N,N- | | |
| bis(cyclopropylmethyl)-8- | | |
| methylquinolin-2-amine | | |
| Hydroxypropyl methyl cellulose | 18.52 | — |
| acetate succinate (AQOAT ®LF) | | |
| Hydroxypropyl methyl cellulose | — | 21.6 |
| acetate succinate (AQOAT ®MF) | | |
| Polyoxyl 35 castor oil | 1.85 | 2.16 |
| (CREMOPHOR ® EL) | | |
| Talc | 3.7 | 4.32 |
| Sugar spheres | 16.67 | 9.72 |
| Acetone | q.s. | q.s. |
| Water | q.s. | q.s. |
| Microcrystalline cellulose | 5.0 | 5.5 |
| Silicified microcrystalline cellulose | 44.63 | 24.57 |
| Sodium stearyl fumarate | 0.37 | 0.36 |

### Process:

1. 3-(((3,5-bis(trifluoromethyl)benzyl)(2-methyl-2H-tetrazol-5-yl)amino)methyl)-N,N-bis(cyclopropylmethyl)-8-methylquinolin-2-amine was dissolved in acetone to form a clear solution.
2. To the step 1, required quantity of water was added and mixed well.
3. To the step 3, HPMCAS, Polyoxyl 35 castor oil and talc were added to form homogenous suspension.
4. The suspension of step 3 was sprayed over inert sugar spheres and dried.
5. The coated spheres of step 4 were blended with microcrystalline cellulose, silicified microcrystalline cellulose and sodium stearyl fumarate and compressed into tablets using suitable size toolings.

### Example 15

| Ingredients | Percent w/w |
|---|---|
| 3-(((3,5-bis(trifluoromethyl)benzyl)(2-methyl- | 8.0 |
| 2H-tetrazol-5-yl)amino)methyl)-N,N- | |
| bis(cyclopropylmethyl)-8-methylquinolin-2- | |
| amine | |
| Hydroxypropyl methyl cellulose acetate | 24.0 |
| succinate (AQOAT ®MF) | |
| Polyoxyl 35 castor oil (CREMOPHOR ® EL) | 2.4 |
| Talc | 4.8 |
| Sugar spheres | 10.8 |
| Acetone | q.s. |
| Water | q.s. |
| Microcrystalline cellulose | 6.37 |
| Silicified microcrystalline cellulose | 36.12 |
| Placebo granules* | 7.15 |
| Sodium stearyl fumarate | 0.35 |

Note:
Placebo granules were prepared by blending microcrystalline cellulose, silified microcrystalline cellulose and hydroxypropylcellulose, and the blend was granulated using hydroxypropylcellulose solution. The resultant granules were dried and sieved for further use.

### Process:

1. 3-(((3,5-bis(trifluoromethyl)benzyl)(2-methyl-2H-tetrazol-5-yl)amino)methyl)-N,N-bis(cyclopropylmethyl)-8-methylquinolin-2-amine was dissolved in acetone to form a clear solution.
2. To the step 1, required quantity of water was added and mixed well.
3. To the step 3, HPMCAS, Polyoxyl 35 castor oil and talc were added to form homogenous suspension.
4. The suspension of step 3 was sprayed over inert sugar spheres and dried.
5. The coated spheres of step 4 were blended with microcrystalline cellulose, silicified microcrystalline cellulose, placebo granules and sodium stearyl fumarate and compressed into tablets using suitable size toolings.

### Example 16

Examples 5-12 were subjected to dissolution test in 900 mL of simplified simulated intestinal fluid (SSIF) at 39° C. and 25 RPM. The SSIF was prepared by dissolving 44.5 g of sodium dihydrogen phosphate dehydrate, 61.8 g of sodium chloride and 5 ml of TWEEN 80® in 10 liters of water. The SSIF solution was adjusted to have a pH of 6.5 with sodium hydroxide. Samples were withdrawn at designated time points, screened through 10-micron filter analyzed for drug release by UV absorption. The amount of drug released is shown in Table 1 and Table 2 below.

TABLE 1

| Time | Example 5 | Example 6 | Example 7 | Example 8 |
|---|---|---|---|---|
| 30 min | 31 | 30 | 30 | 45 |
| 45 min | 49 | 48 | 44 | 60 |
| 60 min | 60 | 63 | 54 | 69 |
| 90 min | 70 | 81 | 67 | 77 |
| 120 min | 75 | 90 | 76 | 82 |
| 180 min | 81 | 97 | 86 | 89 |
| 240 min | 85 | 100 | 91 | 93 |
| 360 min | 90 | 101 | 99 | 98 |
| 480 min | 94 | 102 | 101 | 101 |

TABLE 2

| Time | Example 9 | Example 10 | Example 11 | Example 12 |
|---|---|---|---|---|
| 30 min | 23 | 18 | 18 | 45 |
| 45 min | 31 | 27 | 28 | 58 |
| 60 min | 38 | 34 | 36 | 70 |
| 90 min | 51 | 47 | 49 | 84 |
| 120 min | 61 | 57 | 59 | 89 |
| 180 min | 75 | 71 | 75 | 92 |
| 240 min | 84 | 80 | 85 | 92 |
| 360 min | 94 | 90 | 95 | 92 |
| 480 min | 98 | 96 | 100 | 93 |

### Example 17

A pharmacokinetic study of the Examples 5, 10 and 11 following single oral administration in six (6) male Beagle dogs was conducted under fed and fasted state. The compositions were administered at a dose level of 200 mg/Kg in a randomized crossover design. At least ten-day washout period was maintained between each dose administration to same six animals. The results are shown in Table 3.

TABLE 3

| | | | | | Food | Food |
|---|---|---|---|---|---|---|
| | Fed state | | Fasted state | | effect | effect |
| | | AUC | | AUC | on | on |
| Composition | Cₘₐₓ (μM) | (μM · h) | Cₘₐₓ (μM) | (μM · h) | AUC_{(0-48 hr)} | Cₘₐₓ |
| Example 6 | 3.97 ± 0.94 | 51.91 ± 20.10 | 1.43 ± 0.82 | 27.31 ± 14.40 | 1.9 | 2.78 |
| Example 11 | 1.78 ± 0.94 | 20.5 ± 11.0 | 1.46 ± 1.33 | 17.3 ± 16.8 | 1.19 | 1.2 |
| Example 12 | 1.71 ± 0.41 | 21.1 ± 3.73 | 0.94 ± 0.77 | 9.81 ± 7.38 | 2.15 | 1.81 |

In one embodiment, various compositions in accordance with the present application can be prepared by substituting 1,3-(((3,5-bis(trifluoromethyl)benzyl)(2-methyl-2H-tetrazol-5-yl)amino)methyl)-N,N-bis(cyclopropylmethyl)-8-methylquinolin-2-amine as described in Examples 1-17 with any one of the following compounds:

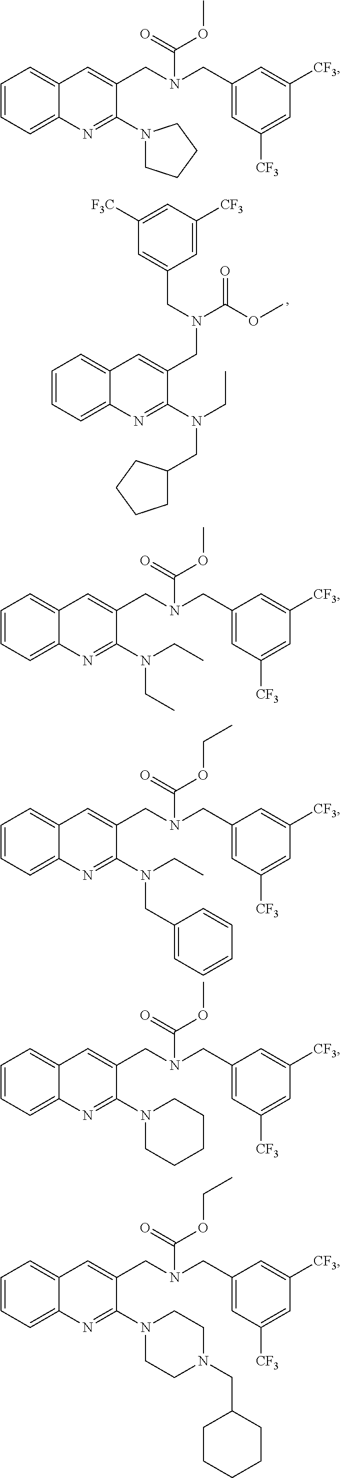

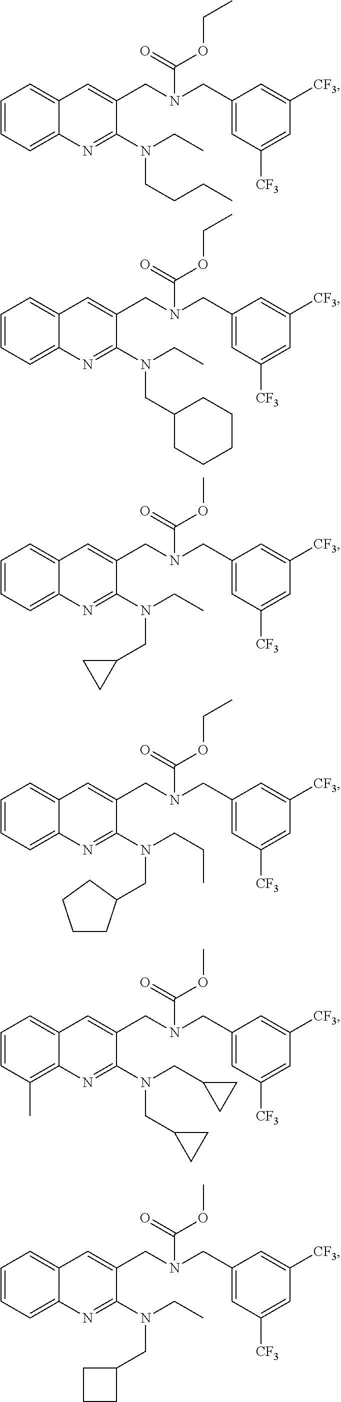

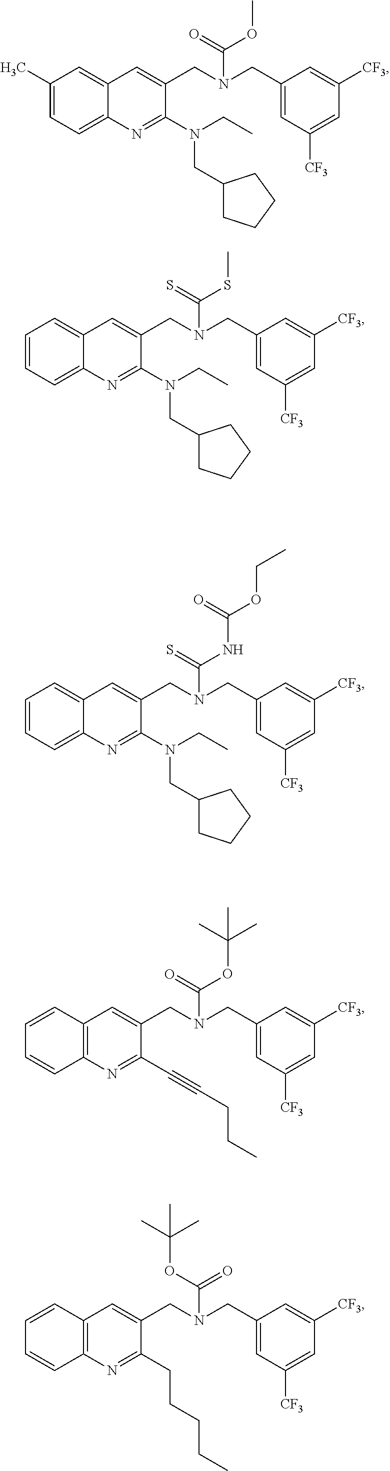

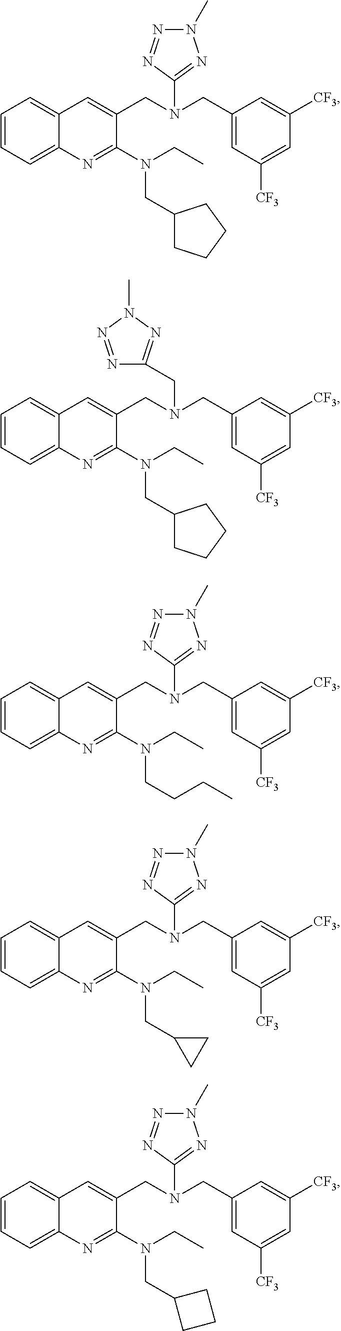

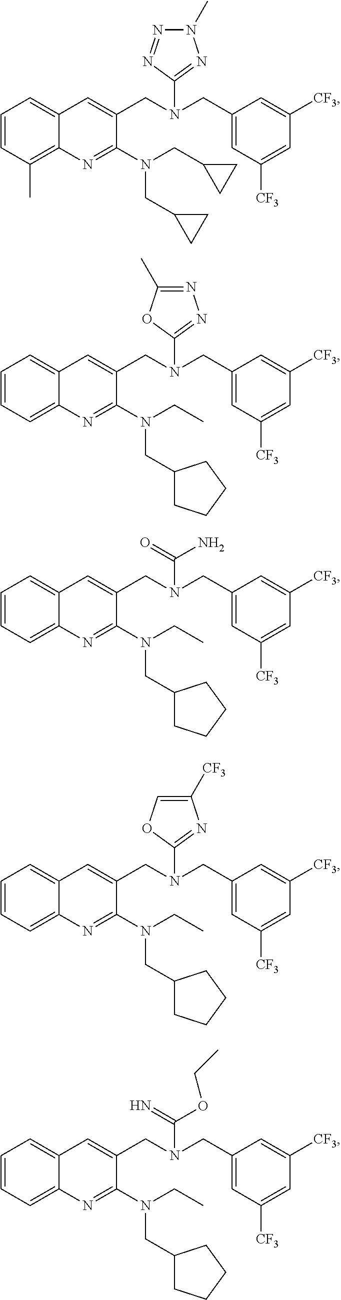

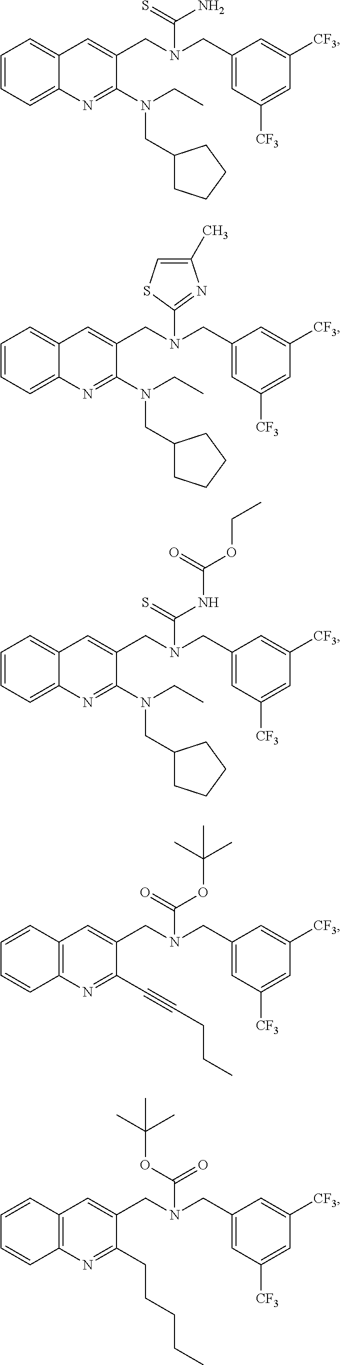

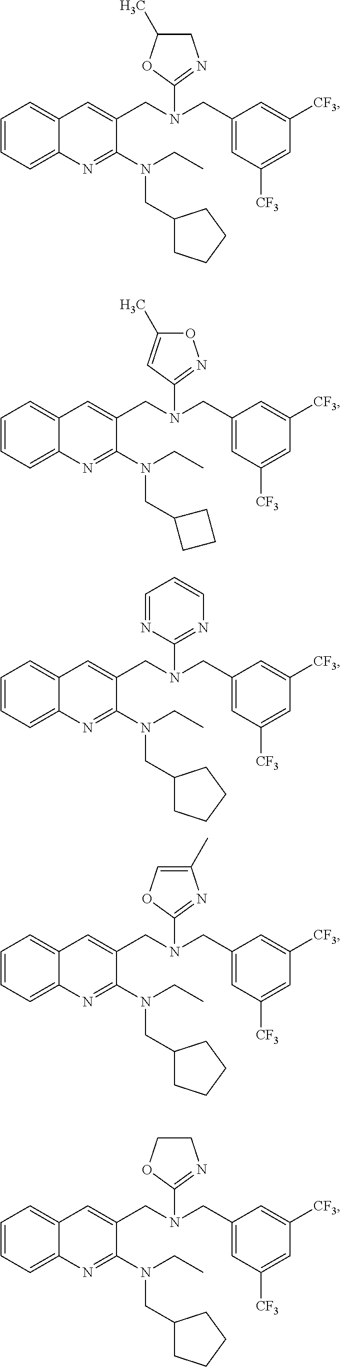

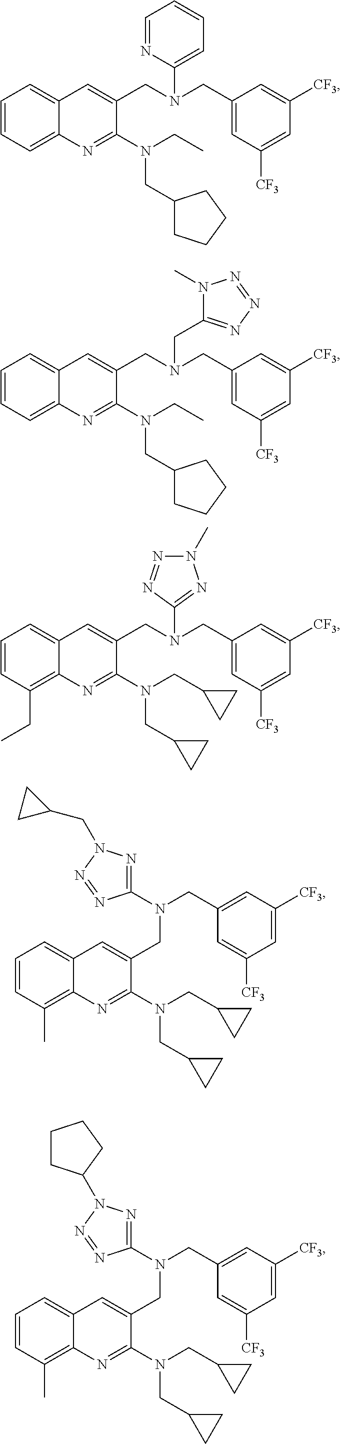

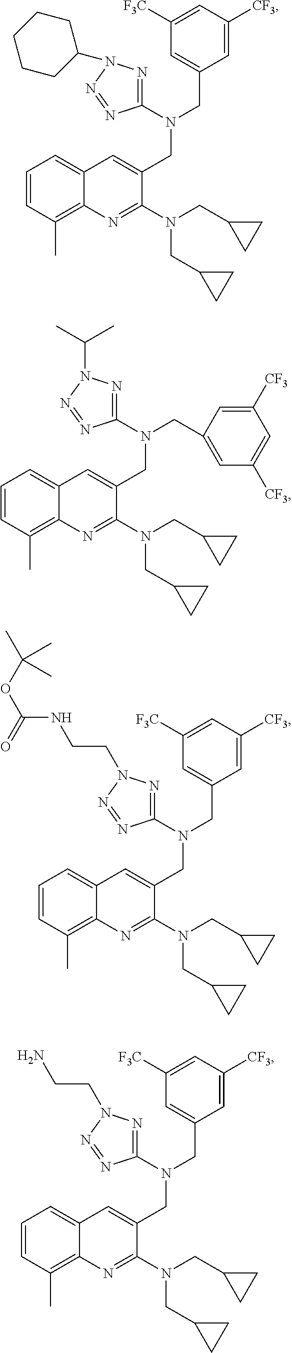 [SMILES: Cc1cccc2cc(CN(Cc3cc(C(F)(F)F)cc(C(F)(F)F)c3)c3nnn(C4CCCCC4)n3)c(N(CC3CC3)CC3CC3)nc12 ; Cc1cccc2cc(CN(Cc3cc(C(F)(F)F)cc(C(F)(F)F)c3)c3nnn(C(C)C)n3)c(N(CC3CC3)CC3CC3)nc12 ; Cc1cccc2cc(CN(Cc3cc(C(F)(F)F)cc(C(F)(F)F)c3)c3nnn(CCNC(=O)OC(C)(C)C)n3)c(N(CC3CC3)CC3CC3)nc12 ; Cc1cccc2cc(CN(Cc3cc(C(F)(F)F)cc(C(F)(F)F)c3)c3nnn(CCN)n3)c(N(CC3CC3)CC3CC3)nc12]

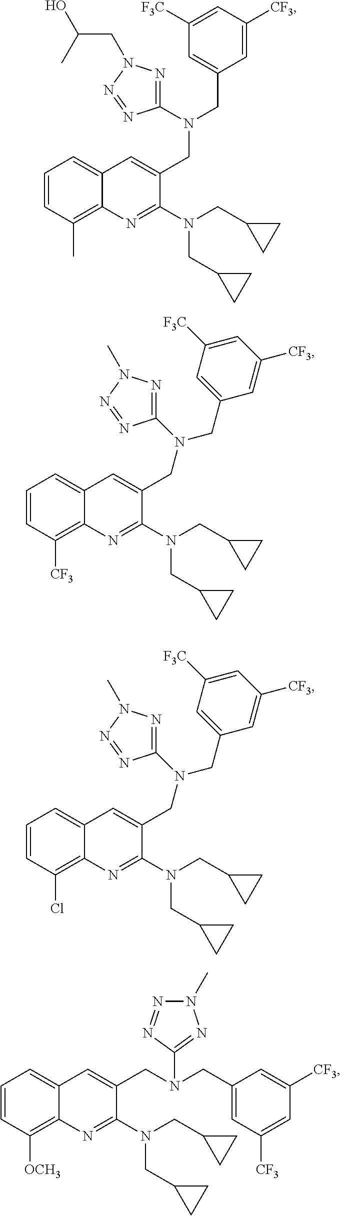

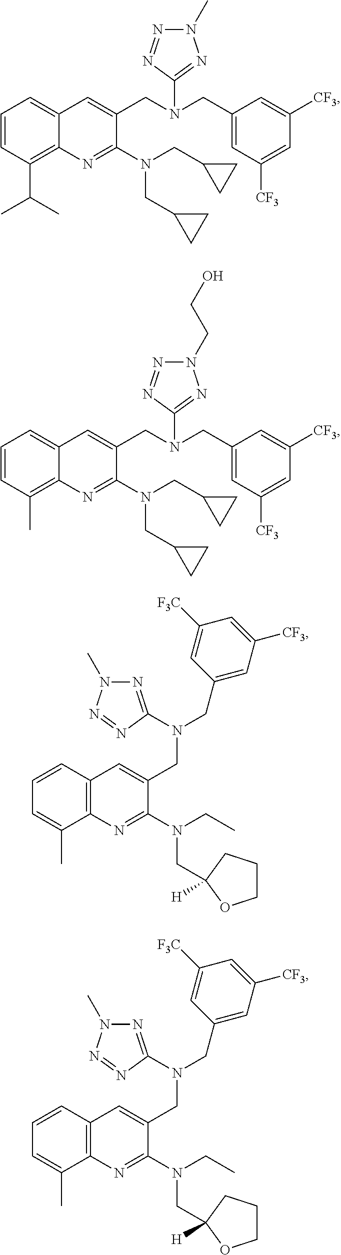

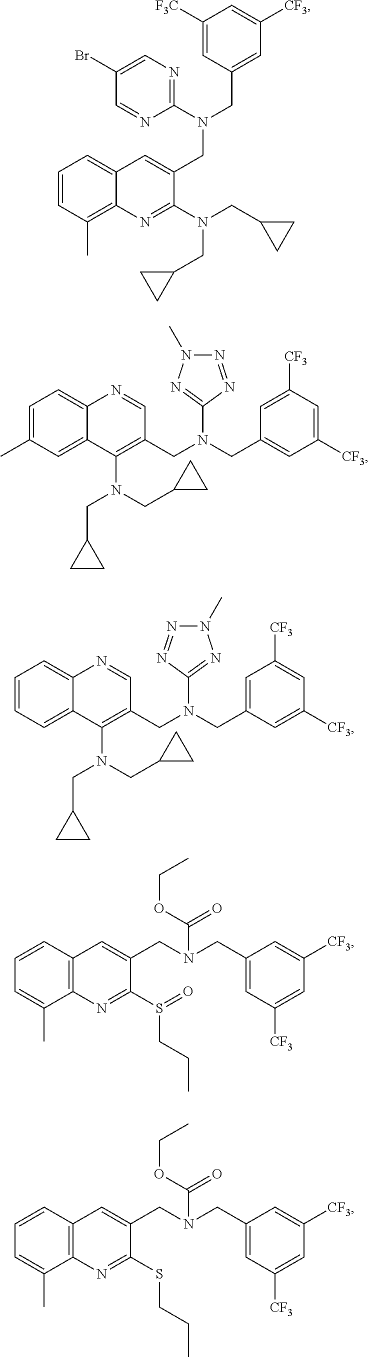

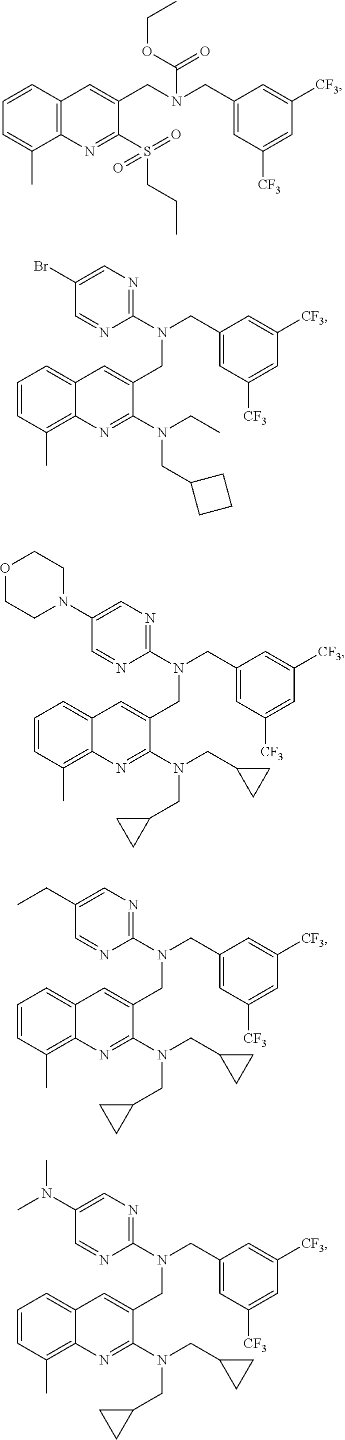 [SMILES: CCCS(=O)(=O)c1nc2c(C)cccc2cc1CN(Cc1cc(C(F)(F)F)cc(C(F)(F)F)c1)C(=O)OCC ; CCN(CC1CCC1)c1nc2c(C)cccc2cc1CN(Cc1cc(C(F)(F)F)cc(C(F)(F)F)c1)c1ncc(Br)cn1 ; Cc1cccc2cc(CN(Cc3cc(C(F)(F)F)cc(C(F)(F)F)c3)c3ncc(N4CCOCC4)cn3)c(N(CC3CC3)CC3CC3)nc12 ; CCc1cnc(N(Cc2cc(C(F)(F)F)cc(C(F)(F)F)c2)Cc2cc3cccc(C)c3nc2N(CC2CC2)CC2CC2)nc1 ; Cc1cccc2cc(CN(Cc3cc(C(F)(F)F)cc(C(F)(F)F)c3)c3ncc(N(C)C)cn3)c(N(CC3CC3)CC3CC3)nc12]

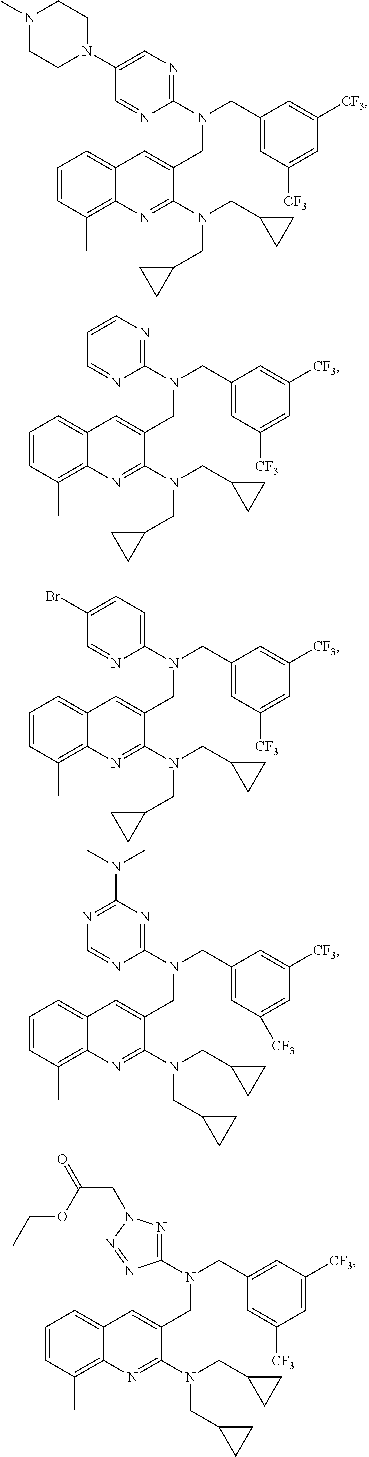

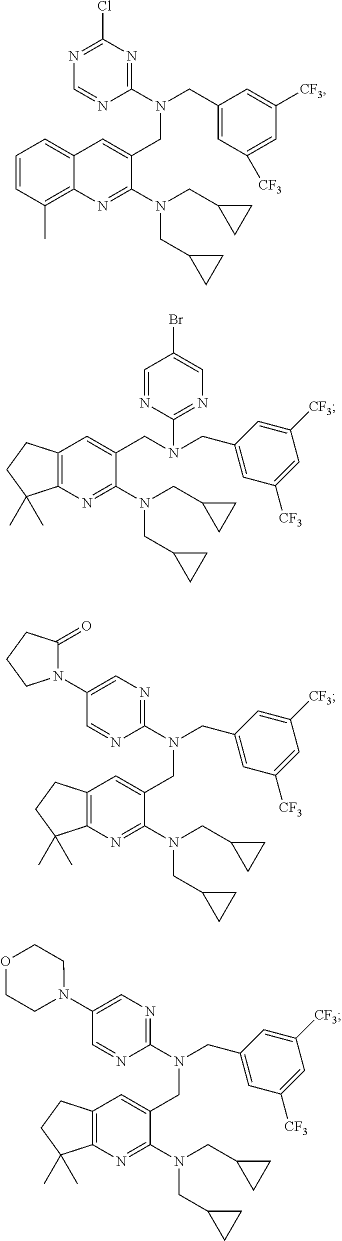 [SMILES: Cc1cccc2cc(CN(Cc3cc(C(F)(F)F)cc(C(F)(F)F)c3)c3ncnc(Cl)n3)c(N(CC3CC3)CC3CC3)nc12 ; CC1(C)CCc2cc(CN(Cc3cc(C(F)(F)F)cc(C(F)(F)F)c3)c3ncc(Br)cn3)c(N(CC3CC3)CC3CC3)nc21 ; CC1(C)CCc2cc(CN(Cc3cc(C(F)(F)F)cc(C(F)(F)F)c3)c3ncc(N4CCCC4=O)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21]

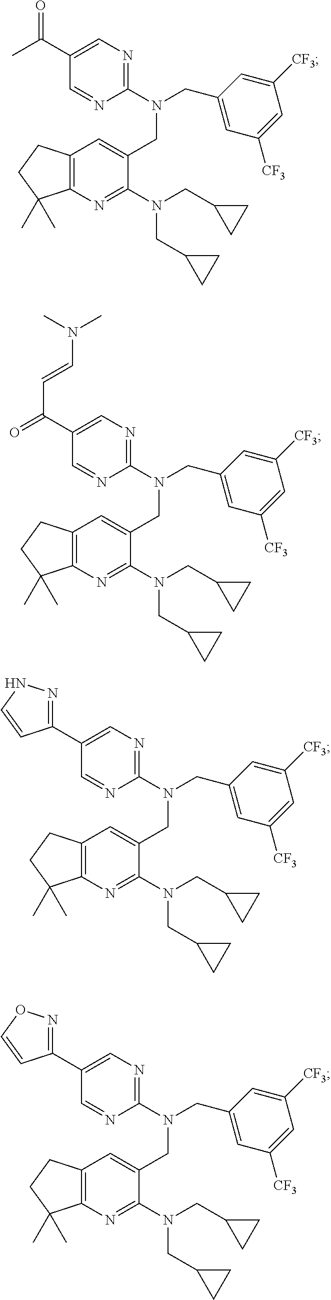 [SMILES: CC(=O)c1cnc(N(Cc2cc(C(F)(F)F)cc(C(F)(F)F)c2)Cc2cc3c(nc2N(CC2CC2)CC2CC2)C(C)(C)CC3)nc1 ; CN(C)/C=C/C(=O)c1cnc(N(Cc2cc(C(F)(F)F)cc(C(F)(F)F)c2)Cc2cc3c(nc2N(CC2CC2)CC2CC2)C(C)(C)CC3)nc1 ; CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(-c4ccon4)cn3)c(N(CC3CC3)CC3CC3)nc21]

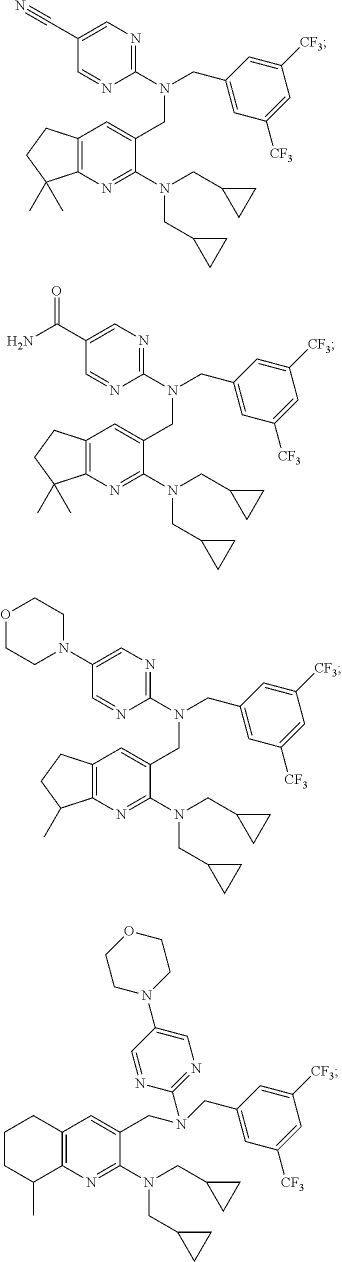 [SMILES: CC1(C)CCc2cc(CN(Cc3cc(C(F)(F)F)cc(C(F)(F)F)c3)c3ncc(C#N)cn3)c(N(CC3CC3)CC3CC3)nc21 ; CC1(C)CCc2cc(CN(Cc3cc(C(F)(F)F)cc(C(F)(F)F)c3)c3ncc(C(N)=O)cn3)c(N(CC3CC3)CC3CC3)nc21 ; CC1CCc2cc(CN(Cc3cc(C(F)(F)F)cc(C(F)(F)F)c3)c3ncc(N4CCOCC4)cn3)c(N(CC3CC3)CC3CC3)nc21 ; CC1CCCc2cc(CN(Cc3cc(C(F)(F)F)cc(C(F)(F)F)c3)c3ncc(N4CCOCC4)cn3)c(N(CC3CC3)CC3CC3)nc21]

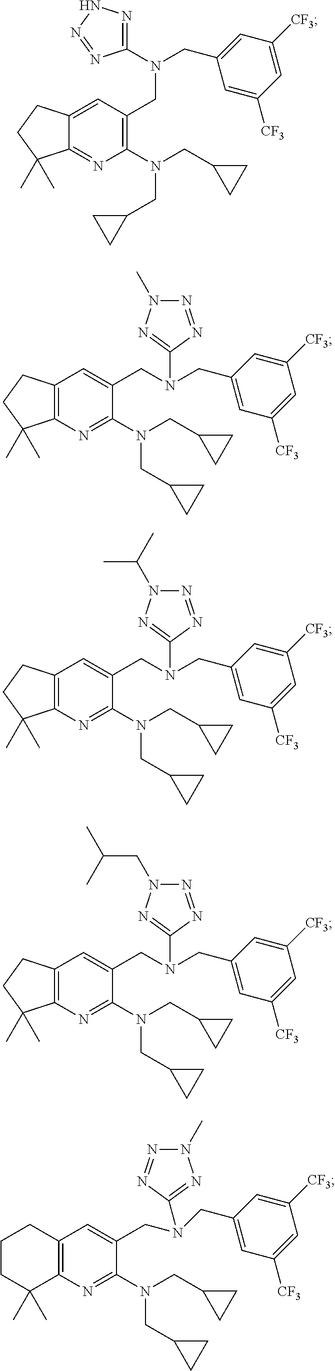

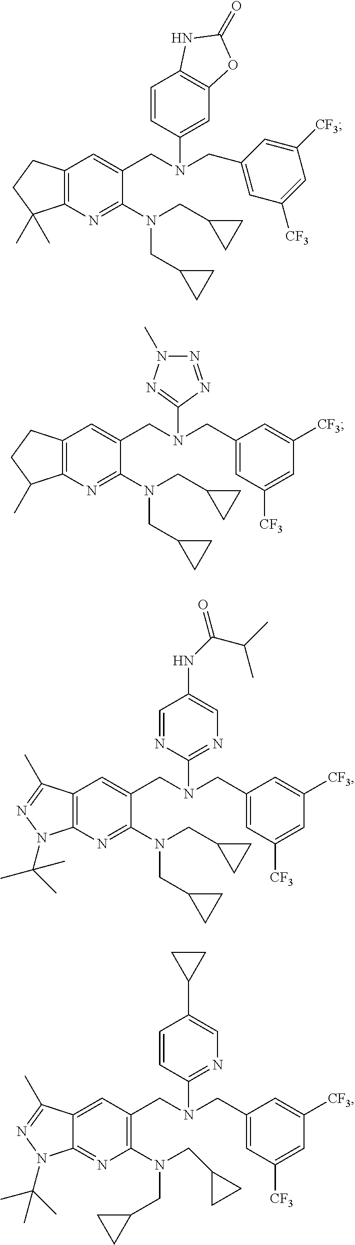

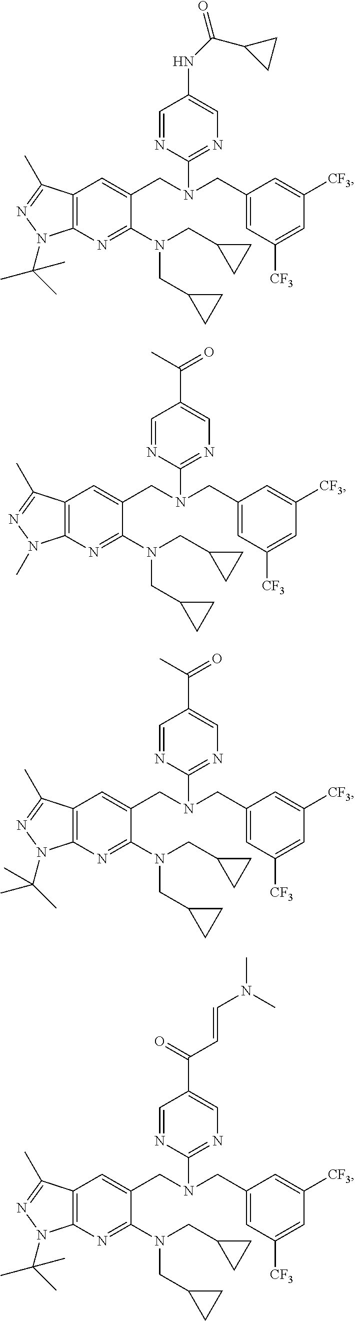

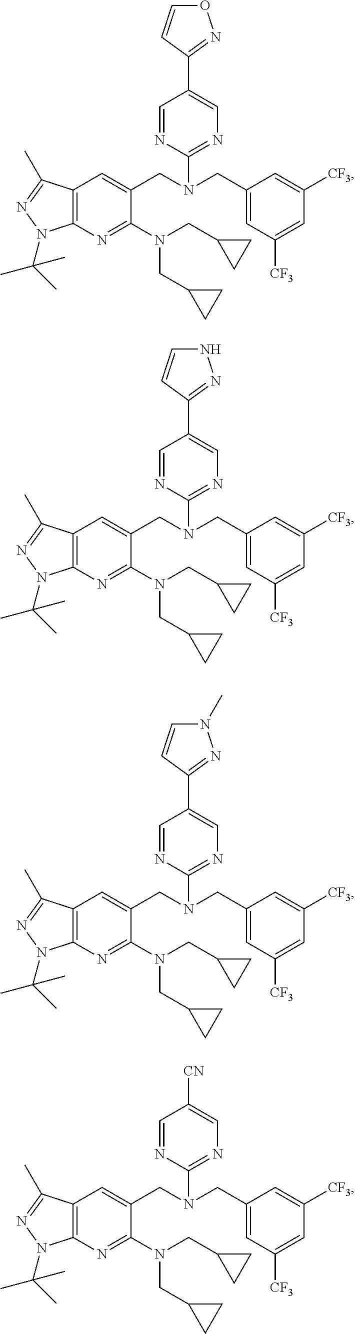

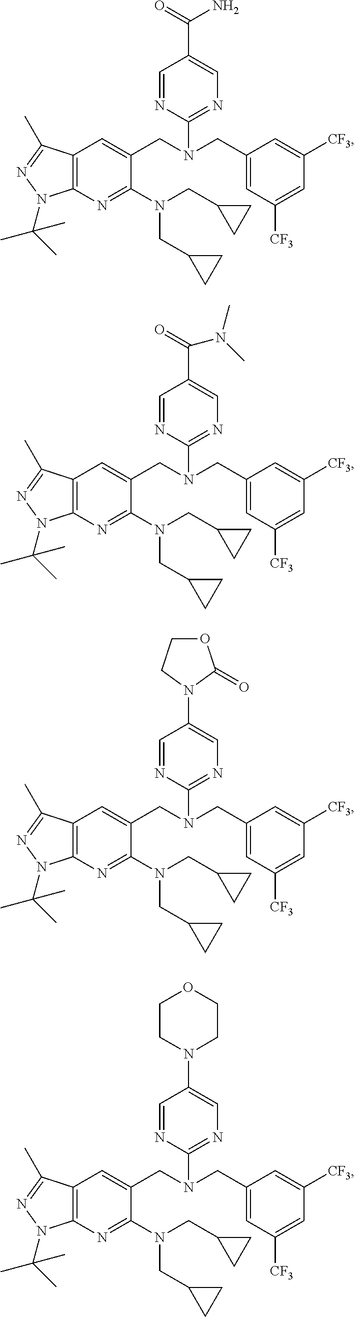

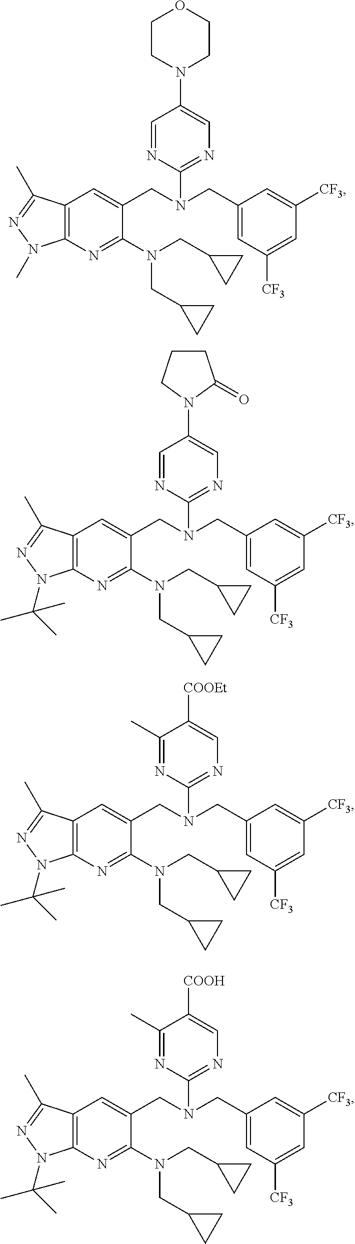

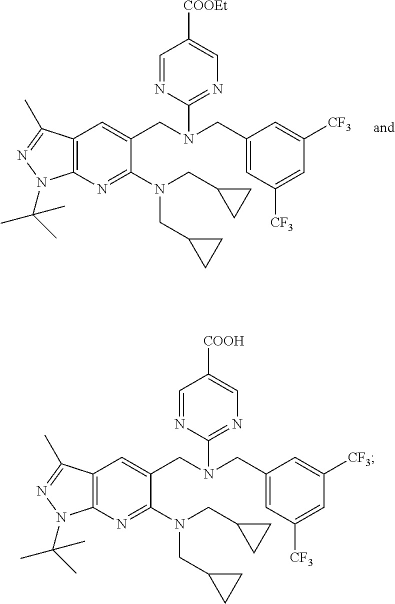 [SMILES: CCOC(=O)c1cnc(N(Cc2cc(C(F)(F)F)cc(C(F)(F)F)c2)Cc2cc3c(C)nn(C(C)(C)C)c3nc2N(CC2CC2)CC2CC2)nc1 ; Cc1nn(C(C)(C)C)c2nc(N(CC3CC3)CC3CC3)c(CN(Cc3cc(C(F)(F)F)cc(C(F)(F)F)c3)c3ncc(C(=O)O)cn3)cc12]

or a stereoisomer thereof or a pharmaceutically acceptable salt thereof.
In another embodiment, various compositions in accordance with the present application can be prepared by substituting 1,3-(((3,5-bis(trifluoromethyl)benzyl)(2-methyl-2H-tetrazol-5-yl)amino)methyl)-N,N-bis(cyclopropylmethyl)-8-methylquinolin-2-amine as described in Examples 1-17 with any one of the following compounds:

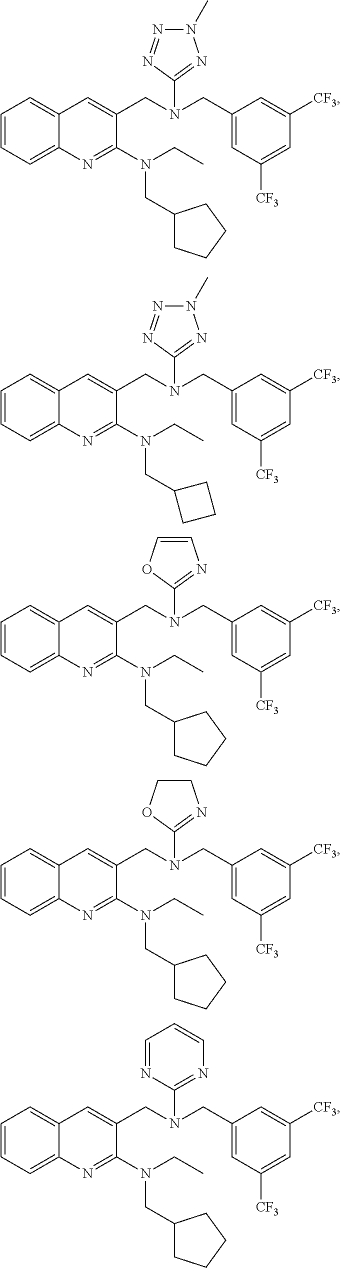

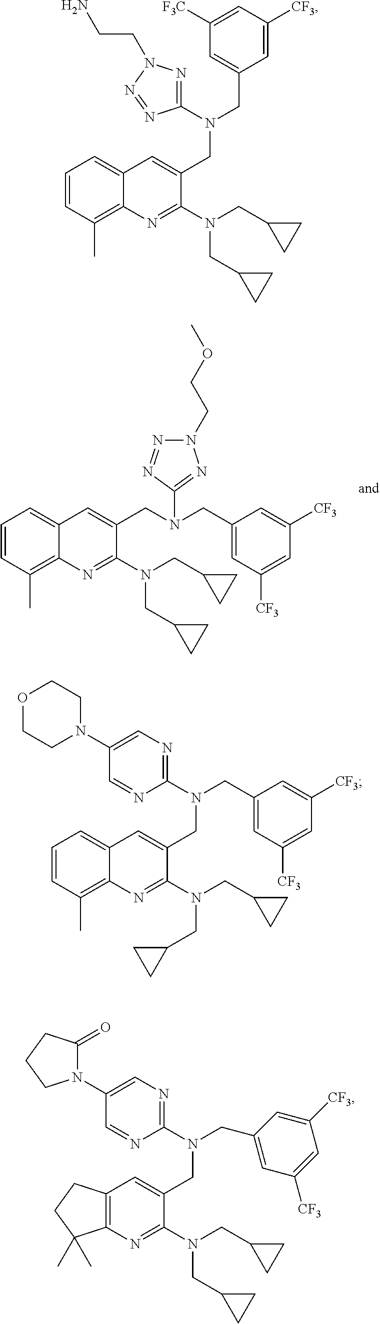 [SMILES: Cc1cccc2cc(CN(Cc3cc(C(F)(F)F)cc(C(F)(F)F)c3)c3nnn(CCN)n3)c(N(CC3CC3)CC3CC3)nc12 ; COCCn1nnc(N(Cc2cc(C(F)(F)F)cc(C(F)(F)F)c2)Cc2cc3cccc(C)c3nc2N(CC2CC2)CC2CC2)n1 ; Cc1cccc2cc(CN(Cc3cc(C(F)(F)F)cc(C(F)(F)F)c3)c3ncc(N4CCOCC4)cn3)c(N(CC3CC3)CC3CC3)nc12 ; CC1(C)CCc2cc(CN(Cc3cc(C(F)(F)F)cc(C(F)(F)F)c3)c3ncc(N4CCCC4=O)cn3)c(N(CC3CC3)CC3CC3)nc21]

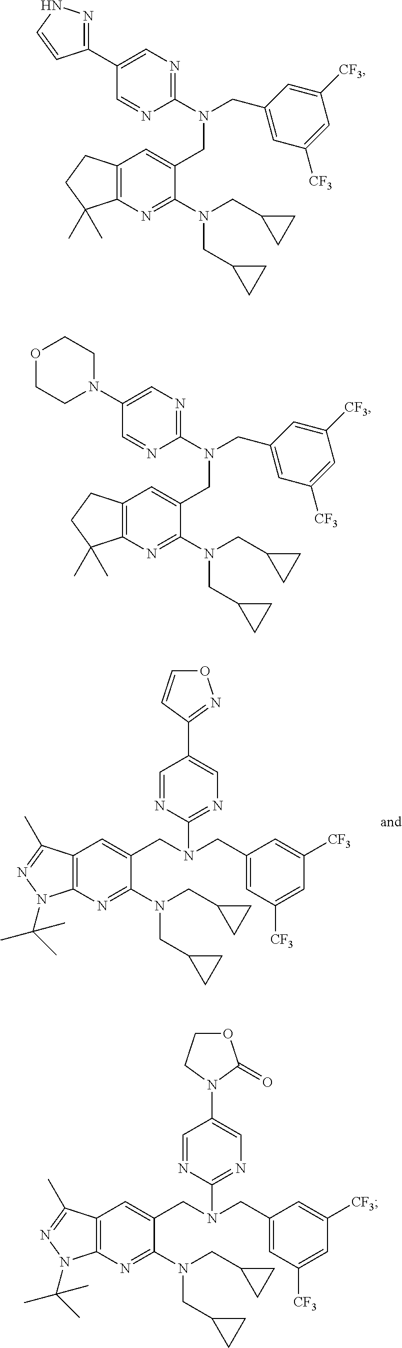 [SMILES: CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; Cc1nn(C(C)(C)C)c2nc(N(CC3CC3)CC3CC3)c(CN(Cc3cc(C(F)(F)F)cc(C(F)(F)F)c3)c3ncc(-c4ccon4)cn3)cc12 ; Cc1nn(C(C)(C)C)c2nc(N(CC3CC3)CC3CC3)c(CN(Cc3cc(C(F)(F)F)cc(C(F)(F)F)c3)c3ncc(N4CCOC4=O)cn3)cc12]

or a stereoisomer thereof or a pharmaceutically acceptable salt thereof.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

1. A pharmaceutical composition comprising:
a) a cholesteryl ester transfer protein (CETP) inhibitor having formula (I) or (Ia′) or (II) or (III),
b) at least one solubility improving material,
c) optionally one or more wetting agents, and
d) at least one pharmaceutically acceptable excipient, wherein
(i) the CETP inhibitor having formula (I) is: 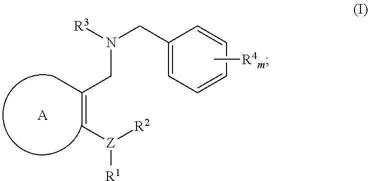
or a stereoisomer thereof or a pharmaceutically acceptable salt thereof, wherein:
A is a substituted or an unsubstituted quinoline moiety having the formula: 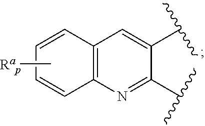
wherein R^{a}, in each occurrence, is selected independently from: 1) a halogen; a hydroxyl, or a cyano; 2) an alkyl or an alkoxy, any of which having up to 12 carbon atoms; or 3) CO₂R⁶; and p is an integer from 0 to 3, inclusive;
R¹ and R² are selected independently from: 1) hydrogen; 2) a substituted or an unsubstituted alkyl, cycloalkyl, haloalkyl, aryl, heterocyclyl, heteroaryl, any of which having up to 12 carbon atoms, wherein any heterocyclyl or heteroaryl comprises at least one heteroatom or heterogroup selected independently from O, N, S, NR¹⁰, SO₂, or CO; 3) CO₂R⁶, COR⁸, SO₂R⁸, SO₂NR⁶R⁷, or CONR⁶R⁷; or 4) (CHR^{x})ₙR⁵ or (CH₂)ₙR^{d}CO₂R^{e}, wherein n, in each occurrence, is 1, 2, or 3; R^{x}, in each occurrence, is selected independently from an alkyl or an alkoxy, either of which having up to 12 carbon atoms, or hydrogen; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, NR¹⁰, SO₂, or CO; and R^{e}, in each occurrence, is selected independently from an alkyl or a cycloalkyl, either of which having up to 12 carbon atoms, or hydrogen;
or R¹ and R² together with the diradical Z to which they are attached_form a substituted or an unsubstituted monocyclic or bicyclic moiety comprising up to 12 carbon atoms, and optionally comprising 1, 2, or 3 heteroatoms or heterogroups in addition to Z, selected independently from O, N, S, NR¹⁰, SO₂, or CO;
R³ is selected from: 1) hydrogen or cyano; 2) a substituted alkyl having up to 12 carbon atoms; 3) a substituted or an unsubstituted aryl, or a substituted or an unsubstituted 5-, 6-, or 7-membered heterocyclyl or heteroaryl, any of which having up to 12 carbon atoms, comprising 1, 2, or 3 heteroatoms or heterogroups selected independently from O, N, S, NR¹⁰, SO₂, or CO; or 4) CO₂R⁶, COR⁸, SO₂R⁸, SO₂NR⁶R⁷, CONR⁶R⁷, C(S)NR⁶R⁷, C(S)NC(O)OR⁸, or C(S)SR⁸; or 5) a substituted or an unsubstituted group selected from 4,5-dihydro-oxazolyl, tetrazolyl, isoxazolyl, pyridyl, pyrimidinyl, oxadiazolyl, thiazolyl, or oxazolyl; wherein any optional substituent is selected independently from: a) an alkyl or haloalkyl, any of which having up to 12 carbon atoms; or b) CO₂R⁹, wherein R⁹ is an alkyl having up to 12 carbon atoms;
wherein when R³ is an aryl, a heterocyclyl, or a heteroaryl, R³ is optionally substituted with up to three substituents selected independently from a halogen, a hydroxyl, a cyano, an alkoxy having up to 12 carbon atoms, or R¹¹;
R⁴, in each occurrence, is selected independently from: 1) halogen, cyano, or hydroxy; 2) an alkyl, a cycloalkyl, a cycloalkoxy, an alkoxy, a haloalkyl, or a haloalkoxy, any of which having up to 12 carbon atoms; 3) a substituted or an unsubstituted aryl, aralkyl, aryloxy, heteroaryl, or heteroaryloxy, any of which having up to 12 carbon atoms, wherein any heteroaryl or heteroaryloxy comprises at least one heteroatom or heterogroup selected independently from O, N, S, or NR¹⁰; or 4) CO₂R⁶, COR⁸, SO₂R⁸, SO₂NR⁶R⁷, CONR⁶R⁷, or (CH₂)NR⁶R⁷, wherein q is an integer from 0 to 5, inclusive;
m is an integer from 0 to 3, inclusive;
or R⁴ₘ is a fused cyclic moiety comprising from 3 to 5 additional ring carbon atoms, inclusive, and optionally comprising at least one heteroatom or heterogroup selected independently from O, N, S, NR¹⁰, SO₂, or CO;
R⁵, in each occurrence, is selected independently from: 1) an alkoxy, a haloalkoxy, or a cycloalkyl, any of which having up to 12 carbon atoms; 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO; 3) hydroxyl, NR⁶R⁷, CO₂R⁶, COR⁸, or SO₂R⁸; or 4) a substituted or an unsubstituted heterocycloalkyl comprising from 3 to 7 ring carbon atoms, and from 1 to 3 heteroatoms or heterogroups, inclusive, selected independently from O, N, S, NR¹⁰, SO₂, or CO;
R⁶ and R⁷, in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
or R⁶ and R⁷ together with the nitrogen atom to which they are attached form a substituted or an unsubstituted cyclic moiety having from 3 to 7 ring carbon atoms, and optionally comprising 1, 2, or 3 heteroatoms in addition to the nitrogen atom to which R⁶ and R⁷ are bonded, selected independently from O, N, S, or NR¹⁰;
R⁸, in each occurrence, is selected independently from: 1) an alkyl, a cycloalkyl, or a haloalkyl, any of which having up to 12 carbon atoms; or 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
Z is N or CH; or the ZR¹ moiety is S, CO, or SO₂; or the ZR¹R² moiety is —C≡CR²;
R¹¹ is selected independently from:
1) an alkyl, a haloalkyl, a cycloalkyl, or an alkoxycarbonyl, any of which having up to 12 carbon atoms;
2) a substituted or an unsubstituted heteroaryl or heterocyclyl, any of which having up to 12 carbon atoms, comprises at least one heteroatom or heterogroup selected independently from O, N, S, NR¹⁰, SO₂, or CO, wherein any substituted heteroaryl or heterocyclyl is substituted with up to three substituents selected independently from an alkyl having up to 12 carbon atoms or a hydroxyl; or
3) —CO—Z²—R¹³, —CO—R¹², —CO—Z²—(CH₂)ᵣ—CO—Z²—R¹³, —NR¹⁵R¹⁶, (—Z²—CO—(CH₂)ᵣ—Z²—R¹³, —Z²—CO—(CH₂)ᵣ—CO—Z²—R¹³, —O—(CH₂)ᵣ—CO—Z²—R¹³, —O—(CH₂)ᵣ—R¹⁴, —O—R¹²—(CH₂)ᵣ—R¹³, —O—R¹⁴—CO—O—R¹³, —O—(CH₂)ᵣ—R¹², —O—(CH₂)ᵣ—NR′R″, —O—(CH₂)ᵣ—CO₂—(CH₂)ᵣ—R¹³, —O—(CH₂)ᵣ—SR⁸, —O—(CH₂)ᵣ—CO₂—R¹³, —O—(CH₂)ᵣ—CONR′R″, —O—(CH₂)ᵣ—CONH—(CH₂)ᵣ—OR¹³, —O—(CH₂)ᵣ—SO₂R⁸, —O—(CH₂)ᵣ—R¹³, —O—(CH₂)ᵣ—OR¹³, —O—(CH₂)ᵣ—O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″, —(CH₂)ᵣ—O—CO—R⁸, —(CH₂)ᵣ—R¹², —(CH₂)ᵣ—R¹³, —(CH₂)ᵣ—CO—Z²—R¹³, —(CH₂)ᵣ—Z²—R¹³, or -alkenylene-CO₂—(CH₂)ᵣ—R¹³;
r, in each occurrence, is independently 1, 2, or 3;
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl, any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
Z², in each occurrence, is selected independently from NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹², —CO₂—(CH₂)ᵣ—CO—Z²—R¹³, —CO₂—(CH₂)ᵣ—OR¹³, —CO—(CH₂)ᵣ—O—(CH₂)ᵣ—O—(CH₂)ᵣ—R¹³, —CO—(CH₂)ᵣ—O(CH₂)ᵣ—OR¹³, or —CO—NH—(CH₂)ᵣ—OR¹³;
or R¹⁵ and R¹⁶ together with the nitrogen atom to which they are attached form a substituted or an unsubstituted cyclic moiety comprising up to 12 carbon atoms, optionally comprising at least one additional heteroatom or heterogroup selected independently from O, N, S, NR¹⁰, SO₂, or CO; wherein any substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰; or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂R⁸, —SO₂NR′R″, or —NR′R″;
wherein the —(CH₂)ᵣ— linking moiety, in any occurrence, is optionally substituted with at least one group selected independently from hydroxyl, amino, or an alkyl having up to 3 carbon atoms;
when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl, any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
when R¹ and R² together with the diradical Z to which they are attached_form a monocyclic or a bicyclic moiety, the cyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl or a heteroaryloxy, any of which having up to 12 carbon atoms, wherein any heteroaryl or heteroaryloxy comprises at least one heteroatom or heterogroup selected independently from O, N, S, or NR¹⁰; or 3) CO₂R⁶, COR⁸, SO₂R⁸, SO₂NR⁶R⁷, or CONR⁶R⁷;
R⁴, R⁶, R⁷, and R⁸ are optionally substituted with at least one substituent, and when substituted, the substituents are selected independently from: 1) halogen, hydroxy, cyano, or NR⁶R⁷; or 2) an alkyl or an alkoxy, any of which having up to 12 carbon atoms;
and R⁵ is optionally substituted with at least one substituent, and when substituted, the substituents are selected independently from: 1) halogen, hydroxy, cyano, or NR⁶R⁷; or 2) an alkyl having up to 12 carbon atoms;
(ii) the CETP inhibitor having formula (Ia′), 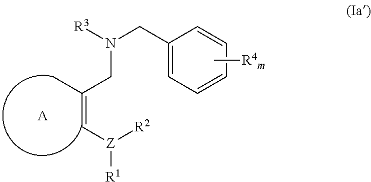
or a stereoisomer thereof or a pharmaceutically acceptable salt thereof, wherein A-ZR¹R² is: 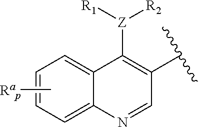
wherein R^{a}, in each occurrence, is selected independently from: 1) a hydrogen, a halogen, a cyano, or a hydroxyl; 2) an alkyl, a haloalkyl, a cycloalkyl, a (cycloalkyl)alkyl, an alkoxy, a cycloalkoxy a haloalkoxy an aryl an aralkyl, a heteroaryl or a heterocyclyl, any of which having up to 12 carbon atoms, wherein any heteroaryl or heterocyclyl, comprises at least one heteroatom or heterogroup selected independently from O, N, S, NR¹⁰, SO₂, or CO; 3) CO₂R⁶, COR⁸, NR⁶R⁷ or SO₂R⁸;
p is an integer from 0 to 3, inclusive;
Z is N or CH; or the ZR¹ moiety is S, SO, CO, or SO₂; or the ZR¹R² moiety is C≡CR² or —C(O)Z³R^{f}, wherein R^{f} is an alkyl, a cycloalkyl, or a (cycloalkyl)alkyl, any of which having up to 12 carbon atoms, or hydrogen; and Z³ is O or NR^{k}, wherein R^{k} is an alkyl, a cycloalkyl, or a (cycloalkyl)alkyl, any of which having up to 12 carbon atoms, or hydrogen;
R¹ and R² are selected independently from: 1) hydrogen; 2) an alkyl having up to 6 carbon atoms; 3) a cycloalkyl having up to 6 carbon atoms; 4) COR⁸; or 5) (CH₂)ₙR⁵ or (CH₂)ₙR^{d}CO₂R^{e}; wherein n, in each occurrence, is 1 or 2; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, SO₂, or CO; and R^{e}, in each occurrence, is selected independently from an alkyl or a cycloalkyl, either of which having up to 12 carbon atoms, or hydrogen:
or R¹ and R² together form a substituted or an unsubstituted monocyclic or bicyclic moiety comprising up to 12 carbon atoms, and optionally comprising 1 or 2 heteroatoms or heterogroups selected independently from O, N, or NR¹⁰; wherein any optional substituent on the cyclic moiety selected from: 1) a cycloalkyl having up to 6 carbon atoms; or 2) an alkyl having up to 2 carbon atoms:
R³ is selected from: 1) cyano; 2) a substituted or an unsubstituted alkyl having up to 12 carbon atoms; 3) a substituted or an unsubstituted aryl, or a substituted or an unsubstituted 5-, 6-, or 7-membered heterocyclyl or heteroaryl, comprising 1, 2, or 3 heteroatoms or heterogroups selected independently from O, N, S, NR¹⁰, SO₂, or CO; any of which having up to 12 carbon atoms; or 4) CO₂R⁶, COR⁸, SO₂R⁸, SO₂NR⁶R⁷, CONR⁶R⁷, C(S)NR⁶R⁷, C(═NH)OR⁸, C(S)NHC(O)OR⁸, or C(S)SR⁸; wherein when R³ is an alkyl, an aryl, a heterocyclyl, or a heteroaryl, R³ is optionally substituted with up to three substituents selected independently from R¹¹;
R⁴, in each occurrence, is selected independently from: 1) halogen, hydroxy or cyano; or 2) an alkyl, an alkoxy, a haloalkyl, or a haloalkoxy any of which having up to 4 carbon atoms; and m is an integer from 1-3 inclusive;
R⁵, in each occurrence, is selected independently from: 1) a substituted or an unsubstituted cycloalkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁶ and R⁷, in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁸, in each occurrence, is selected independently from: 1) an alkyl, a cycloalkyl, or a haloalkyl, any of which having up to 12 carbon atoms; or 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
R¹¹ is selected independently from:
1) a halogen, a hydroxyl or a cyano;
2) an alkyl, a haloalkyl, an alkoxy, a cycloalkyl, or an alkoxycarbonyl, any of which having up to 12 carbon atoms;
3) a substituted or an unsubstituted heteroaryl or heterocyclyl, any of which having up to 12 carbon atoms, comprises at least one heteroatom or heterogroup selected independently from O, N, S, NR¹⁰, SO₂, or CO, wherein any substituted heteroaryl or heterocyclyl is substituted with up to three substituents selected independently from an alkyl having up to 12 carbon atoms or a hydroxyl; or
4) —CO—Z²—R¹³, —CO—R¹², —CO—Z²—(CH₂)ᵣ—CO—Z²—R¹³, —NR¹⁵R¹⁶,
—Z²—CO—(CH₂)ᵣ—Z²—R¹³, —Z²—CO—(CH₂)ᵣ—CO—Z²—R¹³, —O—(CH₂)ᵣ—CO—Z²—R¹³,
—O—(CH₂)ᵣ—R¹⁴, —O—R¹²—(CH₂)ᵣ—R¹³, —O—R¹⁴—CO—O—R¹³, —O—(CH₂)ᵣR¹²,
—O—(CH₂)ᵣ—NR′R″, —O—(CH₂)ᵣ—CO₂—(CH₂)ᵣ—R¹³, —O—(CH₂)ᵣ—SR⁸, —O—(CH₂)ᵣ—CO₂—R¹³,
—O—(CH₂)ᵣ—O—(CH₂)ᵣ—OR¹³,
—O—(CH₂)ᵣ—CONR′R″, —O—(CH₂)ᵣ—CONH—(CH₂)ᵣ—OR¹³, —O—(CH₂)ᵣ—SO₂R⁸,
—O—(CH₂)ᵣ—R¹³, —O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″,
—(CH₂)ᵣ—O—CO—R⁸, —(CH₂)ᵣ—R¹², —(CH₂)ᵣ—R¹³, —(CH₂)ᵣ—NH—(CH₂)ᵣ—OR¹³,
—(CH₂)ᵣ—CO—Z²—R¹³, —(CH₂)ᵣ—Z²—R¹³, —(CH₂)ᵣ—NH—CO—Z²—R¹³, or -alkenylene-CO₂—(CH₂)ᵣ—R¹³;
r, in each occurrence, is independently 1, 2 or 3;
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl, any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
Z², in each occurrence, is selected independently from NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹², —CO₂—(CH₂)ᵣ—CO—Z²—R¹³, —CO₂—(CH₂)ᵣ—OR¹³, —CO—(CH₂)ᵣ—O—(CH₂)ᵣ—O—(CH₂)ᵣ—R¹³, —CO—(CH₂)ᵣ—O(CH₂)ᵣ—OR¹³, or —CO—NH—(CH₂)ᵣ—OR¹³;
or R¹⁵ and R¹⁶ together form a substituted or an unsubstituted cyclic moiety comprising up to 12 carbon atoms, optionally comprising at least one additional heteroatom or heterogroup selected independently from O, N, S, NR¹⁰, SO₂, or CO; wherein any substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰; or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂NR′R″, or —NR′R″; and
wherein the —(CH₂)ᵣ— linking moiety, in any occurrence, is optionally substituted with at least one group selected independently from hydroxyl, amino, or an alkyl having up to 3 carbon atoms;
wherein when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl, any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
wherein when R¹ and R² together form a monocyclic or a bicyclic moiety, the monocyclic or bicyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl or a heteroaryloxy, any of which having up to 12 carbon atoms, wherein any heteroaryl or heteroaryloxy comprises at least one heteroatom or heterogroup selected independently from O, N, S, or NR¹⁰; or 3) CO₂R⁶, (CH₂)_{q}COR⁸, SO₂R⁸, SO₂NR⁶R⁷, or CONR⁶R⁷; or 4) (CH₂)_{q}CO₂(CH₂)_{q}CH₃, wherein q is selected independently from an integer from 0 to 3, inclusive; and
R⁴, R⁶, R⁷, and R⁸ are optionally substituted with at least one substituent, and when substituted, the substituents are selected independently from: 1) halogen, hydroxy, cyano, or NR⁶R⁷; or 2) an alkyl or an alkoxy, any of which having up to 12 carbon atoms; and
R⁵ is optionally substituted with at least one substituent selected independently from: 1) halogen, hydroxy, cyano, or NR⁶R⁷; or 2) an alkyl or an alkoxy, any of which having up to 12 carbon atoms; or 3) (CH₂)ₜOR^{j} or (CH₂)ₜCOOR^{j} wherein t is an integer from 1 to 3, inclusive, and R^{j} is hydrogen or alkyl having up to 12 carbon atoms;
(i) the CETP inhibitor having formula (II) is: 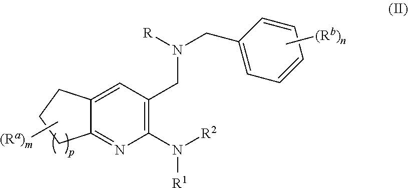
or a stereoisomer thereof or a pharmaceutically acceptable salt thereof; wherein,
R represents 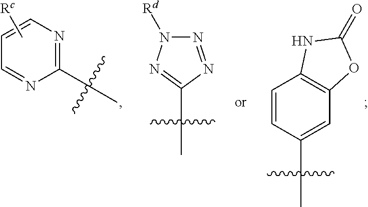
R¹ and R² are independently selected from hydrogen, acyl, haloalkyl, —(CHR^{e})_{q}R³, an optionally substituted group selected from alkyl or cycloalkyl, wherein optional substituent, in each occurrence, is independently selected from halogen, cyano, hydroxyl, an alkyl, a haloalkyl or an alkoxy;
R³ is a group selected from alkoxy, haloalkoxy, cycloalkyl, aryl, heterocyclyl or heteroaryl, wherein R³ is optionally substituted with a group selected from halogen, cyano, hydroxyl, alkyl, haloalkyl or alkoxy;
R^{a}, in each occurrence, is independently selected from halogen, cyano, hydroxy, alkyl, haloalkyl or alkoxy;
R^{b}, in each occurrence, is independently selected from halogen, alkyl, haloalkyl, hydroxy, alkoxy or haloalkoxy;
R^{c} is independently selected from hydrogen, cyano, halogen, —C(═O)—R^{f}, —CONR^{g}R^{h}, —C(═O)—CH≡CH—NRⁱR^{j}, an optionally substituted group selected from cycloalkyl, aryl, heteroaryl or heterocyclyl ring, wherein the optional substituent, in each occurrence, is selected independently from hydrogen, halogen, cyano, hydroxyl, alkyl, haloalkyl, alkoxy, alkoxyalkyl or haloalkoxy;
R^{d} is hydrogen or alkyl;
R^{e}, in each occurrence, is independently selected from hydrogen, alkyl or alkoxy;
R^{f} is hydrogen or alkyl;
R^{g} and R^{h} independently represent hydrogen or alkyl;
Rⁱ and R^{j} independently represent hydrogen or alkyl;
m is 0, 1 or 2;
n is 0, 1, 2 or 3;
p is 1 or 2; and
q is 0, 1, 2, 3, 4 or 5; and
(ii) the CETP inhibitor having formula (III) is: 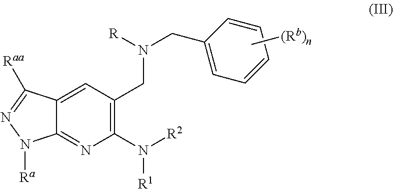
or a stereoisomer thereof or a pharmaceutically acceptable salt thereof; wherein,
R represents hydrogen or 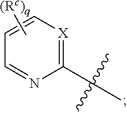
X represents —CH or —N;
R¹ and R² are independently of each other selected from hydrogen, acyl, alkyl or —(CH₂)ₚ-cycloalkyl;
R^{a} and R^{aa} are independently of each other selected from hydrogen or alkyl;
R^{b}, in each occurrence, is independently selected from halogen, alkyl, haloalkyl, hydroxy, alkoxy or haloalkoxy;
R^{c}, in each occurrence, is independently selected from hydrogen, cyano, halogen, alkyl, alkoxy, haloalkoxy, —COOR^{d}, —C(═O)—R^{e}, —CONR^{g}R^{h}, —C(═O)—CH═CH—NRⁱR^{j}, —NHCOR^{t}, an optionally substituted group selected from cycloalkyl, aryl, heteroaryl or heterocycle ring, wherein the optional substituent, in each occurrence, is selected independently from hydrogen, halogen, cyano, hydroxyl, alkyl, haloalkyl, alkoxy, alkoxyalkyl or haloalkoxy;
R^{d}, R^{e}, R^{g}, R^{h}, Rⁱ and R^{j}, in each occurrence, independently of each other represents hydrogen or alkyl;
R^{t} is selected from hydrogen, alkyl or cycloalkyl;
n is 0, 1, 2 or 3;
p is 0, 1, or 2; and
q is 1 or 2.

2. (canceled)

3. The composition according to claim 1, wherein formula (Ia′) is defined as follows 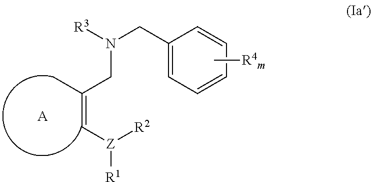
or a stereoisomer thereof or a pharmaceutically acceptable salt thereof, wherein A-ZR¹R² is: 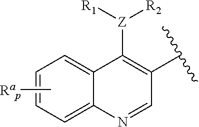
wherein R^{a}, in each occurrence, is selected independently from: 1) a hydrogen, a halogen, a cyano, or a hydroxyl; 2) an alkyl, a haloalkyl, a cycloalkyl, a (cycloalkyl)alkyl, an alkoxy, a cycloalkoxy, a haloalkoxy, an aryl, an aralkyl, a heteroaryl or a heterocyclyl, any of which having up to 12 carbon atoms, wherein any heteroaryl or heterocyclyl, comprises at least one heteroatom or heterogroup selected independently from O, N, S, NR¹⁰, SO₂, or CO; 3) CO₂R⁶, COR⁸, NR⁶R⁷ or SO₂R⁸;
p is an integer from 0 to 3, inclusive;
Z is N or CH; or the ZR¹ moiety is S, SO, CO, or SO₂; or the ZR¹R² moiety is C≡CR² or —C(O)Z³R^{f}, wherein R^{f} is an alkyl, a cycloalkyl, or a (cycloalkyl)alkyl, any of which having up to 12 carbon atoms, or hydrogen; and Z³ is O or NR^{k}, wherein R^{k} is an alkyl, a cycloalkyl, or a (cycloalkyl)alkyl, any of which having up to 12 carbon atoms, or hydrogen;
R¹ and R² are selected independently from: 1) hydrogen; 2) an alkyl having up to 6 carbon atoms; 3) a cycloalkyl having up to 6 carbon atoms; 4) COR⁸; or 5) (CH₂)ₙR⁵ or (CH₂)ₙR^{d}CO₂R^{e}; wherein n, in each occurrence, is 1 or 2; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, NR¹⁰, SO₂, or CO; and R^{e}, in each occurrence, is selected independently from an alkyl or a cycloalkyl, either of which having up to 12 carbon atoms, or hydrogen;
or R¹ and R² together form a substituted or an unsubstituted monocyclic or bicyclic moiety comprising up to 12 carbon atoms, and optionally comprising 1 or 2 heteroatoms or heterogroups selected independently from O, N, or NR¹⁰; wherein any optional substituent on the cyclic moiety selected from: 1) a cycloalkyl having up to 6 carbon atoms; or 2) an alkyl having up to 2 carbon atoms;
R³ is selected from: 1) cyano; 2) a substituted or an unsubstituted alkyl having up to 12 carbon atoms; 3) a substituted or an unsubstituted aryl, or a substituted or an unsubstituted 5-, 6-, or 7-membered heterocyclyl or heteroaryl, comprising 1, 2, or 3 heteroatoms or heterogroups selected independently from O, N, S, NR¹⁰, SO₂, or CO; any of which having up to 12 carbon atoms; or 4) CO₂R⁶, COR⁸, SO₂R⁸, SO₂NR⁶R⁷, CONR⁶R⁷, C(S)NR⁶R⁷, C(═NH)OR⁸, C(S)NHC(O)OR⁸, or C(S)SR⁸; wherein when R³ is an alkyl, an aryl, a heterocyclyl, or a heteroaryl, R³ is optionally substituted with up to three substituents selected independently from R¹¹;
R⁴, in each occurrence, is selected independently from: 1) halogen, hydroxy or cyano; or 2) an alkyl, an alkoxy, a haloalkyl, or a haloalkoxy any of which having up to 4 carbon atoms; and m is an integer from 1-3, inclusive;
R⁵, in each occurrence, is selected independently from: 1) a substituted or an unsubstituted cycloalkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁶ and R⁷, in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁸, in each occurrence, is selected independently from: 1) an alkyl, a cycloalkyl, or a haloalkyl, any of which having up to 12 carbon atoms; or 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
R¹¹ is selected independently from:
1) a halogen, a hydroxyl or a cyano;
2) an alkyl, a haloalkyl, an alkoxy, a cycloalkyl, or an alkoxycarbonyl, any of which having up to 12 carbon atoms;
3) a substituted or an unsubstituted heteroaryl or heterocyclyl, any of which having up to 12 carbon atoms, comprises at least one heteroatom or heterogroup selected independently from O, N, S, NR¹⁰, SO₂, or CO, wherein any substituted heteroaryl or heterocyclyl is substituted with up to three substituents selected independently from an alkyl having up to 12 carbon atoms or a hydroxyl; or
4) —CO—Z²—R¹³, —CO—R¹², —CO—Z²—(CH₂)ᵣ—CO—Z²—R¹³, —NR¹⁵R¹⁶,
—Z²—CO—(CH₂)ᵣ—Z²—R¹³, —Z²—CO—(CH₂)ᵣ—CO—Z²—R¹³, —O—(CH₂)ᵣ—CO—Z²—R¹³,
—O—(CH₂)ᵣ—R¹⁴, —O—R¹²—(CH₂)ᵣ—R¹³, —O—R¹⁴—CO—O—R¹³, —O—(CH₂)ᵣ—R¹²,
—O—(CH₂)ᵣ—NR′R″, —O—(CH₂)ᵣ—CO₂—(CH₂)ᵣ—R¹³, —O—(CH₂)ᵣ—SR⁸, —O—(CH₂)ᵣ—CO₂—R¹³,
—O—(CH₂)ᵣ—O—(CH₂)ᵣ—OR¹³,
—O—(CH₂)ᵣ—CONR′R″, —O—(CH₂)ᵣ—CONH—(CH₂)ᵣ—OR¹³, —O—(CH₂)ᵣ—SO₂R⁸,
—O—(CH₂)ᵣ—R¹³, —O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″,
—(CH₂)ᵣ—O—CO—R⁸, —(CH₂)ᵣ—R¹², —(CH₂)ᵣ—R¹³, —(CH₂)ᵣ—NH—(CH₂)ᵣ—OR¹³,
—(CH₂)ᵣ—CO—Z²—R¹³, —(CH₂)ᵣ—Z²—R¹³, —(CH₂)ᵣ—NH—CO—Z²—R¹³, or -alkenylene-CO₂—(CH₂)ᵣ—R¹³;
r, in each occurrence, is independently 1, 2, or 3;
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl, any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
Z², in each occurrence, is selected independently from NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹², —CO₂—(CH₂)ᵣ—CO—Z²—R¹³, —CO₂—(CH₂)ᵣ—OR¹³, —CO—(CH₂)ᵣ—O—(CH₂)ᵣ—O—(CH₂)ᵣ—R¹³, —CO—(CH₂)ᵣ—O(CH₂)ᵣ—OR¹³, or —CO—NH—(CH₂)ᵣ—OR¹³;
or R¹⁵ and R¹⁶ together form a substituted or an unsubstituted cyclic moiety comprising up to 12 carbon atoms, optionally comprising at least one additional heteroatom or heterogroup selected independently from O, N, S, NR¹⁰, SO₂, or CO; wherein any substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰; or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂R⁸, —SO₂NR′R″, or —NR′R″; and
wherein the —(CH₂)ᵣ— linking moiety, in any occurrence, is optionally substituted with at least one group selected independently from hydroxyl, amino, or an alkyl having up to 3 carbon atoms;
wherein when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl, any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
wherein when R¹ and R² together form a monocyclic or a bicyclic moiety, the monocyclic or bicyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl or a heteroaryloxy, any of which having up to 12 carbon atoms, wherein any heteroaryl or heteroaryloxy comprises at least one heteroatom or heterogroup selected independently from O, N, S, or NR¹⁰; or 3) CO₂R⁶, (CH₂)_{q}COR⁸, SO₂R⁸, SO₂NR⁶R⁷, or CONR⁶R⁷; or 4) (CH₂)_{q}CO₂(CH₂)_{q}CH₃, wherein q is selected independently from an integer from 0 to 3, inclusive; and
R⁴, R⁶, R⁷, and R⁸ are optionally substituted with at least one substituent, and when substituted, the substituents are selected independently from: 1) halogen, hydroxy, cyano, or NR⁶R⁷; or 2) an alkyl or an alkoxy, any of which having up to 12 carbon atoms; and
R⁵ is optionally substituted with at least one substituent selected independently from: 1) halogen, hydroxy, cyano, or NR⁶R⁷; or 2) an alkyl or an alkoxy, any of which having up to 12 carbon atoms; or 3) (CH₂)ₜ—OR^{j} or (CH₂)ₜCOOR^{j} wherein t is an integer from 1 to 3, inclusive, and R^{j} is hydrogen or alkyl having up to 12 carbon atoms.

4. The composition according to claim 1, wherein formula (II) is defined as follows 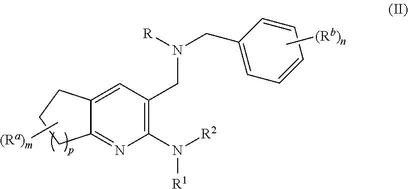
or a stereoisomer thereof or a pharmaceutically acceptable salt thereof; wherein,
R represents 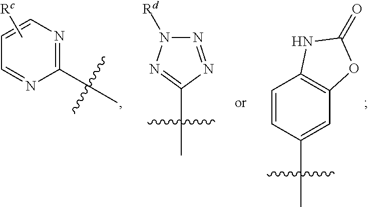
R¹ and R² are independently selected from hydrogen, acyl, haloalkyl, —(CHR^{e})_{q}R³, an optionally substituted group selected from alkyl or cycloalkyl, wherein optional substituent, in each occurrence, is independently selected from halogen, cyano, hydroxyl, an alkyl, a haloalkyl or an alkoxy;
R³ is a group selected from alkoxy, haloalkoxy, cycloalkyl, aryl, heterocyclyl or heteroaryl, wherein R³ is optionally substituted with a group selected from halogen, cyano, hydroxyl, alkyl, haloalkyl or alkoxy;
R^{a}, in each occurrence, is independently selected from halogen, cyano, hydroxy, alkyl, haloalkyl or alkoxy;
R^{b}, in each occurrence, is independently selected from halogen, alkyl, haloalkyl, hydroxy, alkoxy or haloalkoxy;
R^{c} is independently selected from hydrogen, cyano, halogen, —C(═O)—R^{f}, —CONR^{g}R^{h}, —C(═O)—CH≡CH—NRⁱR^{j}, an optionally substituted group selected from cycloalkyl, aryl, heteroaryl or heterocyclyl ring, wherein the optional substituent, in each occurrence, is selected independently from hydrogen, halogen, cyano, hydroxyl, alkyl, haloalkyl, alkoxy, alkoxyalkyl or haloalkoxy;
R^{d} is hydrogen or alkyl;
R^{e}, in each occurrence, is independently selected from hydrogen, alkyl or alkoxy;
R^{f} is hydrogen or alkyl;
R^{g} and R^{h} independently represent hydrogen or alkyl;
Rⁱ and R^{j} independently represent hydrogen or alkyl;
m is 0, 1 or 2;
n is 0, 1, 2 or 3;
p is 1 or 2; and
q is 0, 1, 2, 3, 4 or 5.

5. The composition according to claim 1, wherein formula (III) is defined as follows 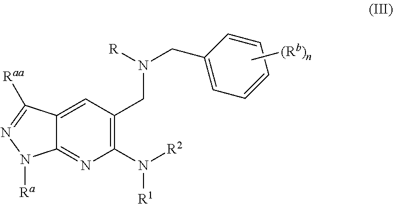
or a stereoisomer thereof or a pharmaceutically acceptable salt thereof, wherein,
R represents hydrogen or 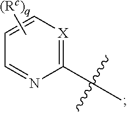
X represents —CH or —N;
R¹ and R² are independently of each other selected from hydrogen, acyl, alkyl or —(CH₂)ₚ-cycloalkyl;
R^{a} and R^{aa} are independently of each other selected from hydrogen or alkyl;
R^{b}, in each occurrence, is independently selected from halogen, alkyl, haloalkyl, hydroxy, alkoxy or haloalkoxy;
R^{c}, in each occurrence, is independently selected from hydrogen, cyano, halogen, alkyl, alkoxy, haloalkoxy, —COOR^{d}, —C(═O)—R^{e}, —CONR^{g}R^{h}, —C(═O)—CH═CH—NRⁱR^{j}, —NHCOR^{t}, an optionally substituted group selected from cycloalkyl, aryl, heteroaryl or heterocycle ring, wherein the optional substituent, in each occurrence, is selected independently from hydrogen, halogen, cyano, hydroxyl, alkyl, haloalkyl, alkoxy, alkoxyalkyl or haloalkoxy;
R^{d}, R^{e}, R^{g}, R^{h}, Rⁱ and R^{j}, in each occurrence, independently of each other represents hydrogen or alkyl;
R^{t} is selected from hydrogen, alkyl or cycloalkyl;
n is 0, 1, 2 or 3;
p is 0, 1, or 2; and
q is 1 or 2.

6. The composition according to claim 1, wherein the solubility improving material is non-ionizable cellulosic polymer, comprising from about 5% w/w to about 80% w/w of the composition and is selected from the group consisting of hydroxypropyl methyl cellulose acetate, hydroxypropyl methyl cellulose, hydroxypropyl cellulose, methyl cellulose, hydroxyethyl methyl cellulose, hydroxyethyl cellulose acetate, hydroxypropyl cellulose acetate, hydroxyethyl ethyl cellulose, and combinations thereof.

7. The composition according to claim 1, wherein the solubility improving material is ionizable cellulosic polymer, comprising from about 5% w/w to about 80% w/w of the composition and is selected from the group consisting of hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, ethyl carboxymethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, ethyl picolinic acid cellulose acetate and combinations comprising one or more of the foregoing materials.

8. (canceled)

9. The composition according to claim 1, wherein the wetting agent(s), comprising up to about 15% w/w of the composition and is selected from the group comprising of fatty acids, alkyl sulfonates, benzalkonium chloride, dioctyl sodium sulfosuccinate (Docusate Sodium) and sodium lauryl sulfate (sodium dodecyl sulfate), sorbitan fatty acid esters, Vitamin E TPGS, polyoxyethylene sorbitan fatty acid esters, polyoxyethylene castor oils, hydrogenated castor oils, sodium taurocholic acid, 1-palmitoyl-2-oleoyl-sn-glycero-3-phosphocholine, lecithin, other phospholipids and mono- and diglycerides, polyoxyethylene fatty acid glycerides, stearyl alcohol, cetostearyl alcohol, cholesterol, polyoxyethylene ricin oil, polyethylene glycol glycerides, poloxamers, and combinations comprising one or more of the forgoing materials.

10. (canceled)

11. The composition according to claim 1, wherein the CETP inhibitor is processed with the solubility improving material, in form of solid amorphous dispersion or solid solution or admixture of simple physical mixture.

12. A composition comprising a solid amorphous dispersion of CETP inhibitor having formula (I) or (Ia′) or (II) or (III) and at least one solubility improving material, wherein said solid amorphous dispersion is substantially homogeneously distributed throughout the solubility improving material and wherein
(a) the CETP inhibitor having formula (I) is: 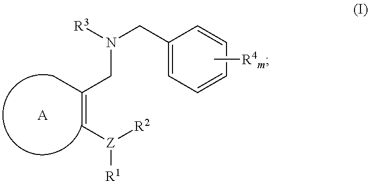
or a stereoisomer thereof or a pharmaceutically acceptable salt thereof, wherein:
A is a substituted or an unsubstituted quinoline moiety having the formula: 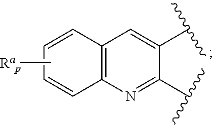
wherein R^{a}, in each occurrence, is selected independently from: 1) a halogen; a hydroxyl, or a cyano; 2) an alkyl or an alkoxy, any of which having up to 12 carbon atoms; or 3) CO₂R⁶; and p is an integer from 0 to 3, inclusive;
R¹ and R² are selected independently from: 1) hydrogen; 2) a substituted or an unsubstituted alkyl, cycloalkyl, haloalkyl, aryl, heterocyclyl, heteroaryl, any of which having up to 12 carbon atoms, wherein any heterocyclyl or heteroaryl comprises at least one heteroatom or heterogroup selected independently from O, N, S, NR¹⁰, SO₂, or CO; 3) CO₂R⁶, COR⁸, SO₂R⁸, SO₂NR⁶R⁷, or CONR⁶R⁷; or 4) (CHR^{x})ₙR⁵ or (CH₂)ₙR^{d}CO₂R^{e}, wherein n, in each occurrence, is 1, 2, or 3; R^{x}, in each occurrence, is selected independently from an alkyl or an alkoxy, either of which having up to 12 carbon atoms, or hydrogen; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, NR¹⁰, SO₂, or CO; and R^{e}, in each occurrence, is selected independently from an alkyl or a cycloalkyl, either of which having up to 12 carbon atoms, or hydrogen;
or R¹ and R² together with the diradical Z to which they are attached form a substituted or an unsubstituted monocyclic or bicyclic moiety comprising up to 12 carbon atoms, and optionally comprising 1, or 3 heteroatoms or heterogroups in addition to Z, selected independently from O, N, S, NR¹⁰, SO₂, or CO;
R³ is selected from: 1) hydrogen or cyano; 2) a substituted alkyl having up to 12 carbon atoms; 3) a substituted or an unsubstituted aryl or a substituted or an unsubstituted 5-, 6-, or 7-membered heterocyclyl or heteroaryl, any of which having up to 12 carbon atoms, comprising 1, 2, or 3 heteroatoms or heterogroups selected independently from O, N, S, NR¹⁰, SO₂, or CO; or 4) CO₂R⁶, COR⁸, SO₂R⁸, SO₂NR⁶R⁷, CONR⁶R⁷, C(S)NR⁶R⁷, C(S)NC(O)OR⁸, or C(S)SR⁸; or 5) a substituted or an unsubstituted group selected from 4,5-dihydro-oxazolyl, tetrazolyl, isoxazolyl, pyridyl, pyrimidinyl, oxadiazolyl, thiazolyl, or oxazolyl; wherein any optional substituent is selected independently from: a) an alkyl or haloalkyl, any of which having up to 12 carbon atoms; or b) CO₂R⁹, wherein R⁹ is an alkyl having up to 12 carbon atoms;
wherein when R³ is an aryl, a heterocyclyl, or a heteroaryl, R³ is optionally substituted with up to three substituents selected independently from a halogen, a hydroxyl, a cyano, an alkoxy having up to 12 carbon atoms, or R¹¹;
R⁴, in each occurrence, is selected independently from: 1) halogen, cyano, or hydroxy; 2) an alkyl, a cycloalkyl, a cycloalkoxy, an alkoxy, a haloalkyl or a haloalkoxy, any of which having up to 12 carbon atoms; 3) a substituted or an unsubstituted aryl, aralkyl, aryloxy, heteroaryl, or heteroaryloxy, any of which having up to 12 carbon atoms, wherein any heteroaryl or heteroaryloxy comprises at least one heteroatom or heterogroup selected independently from O, N, S, or NR¹⁰; or 4) CO₂R⁶, COR⁸, SO₂R⁸, SO₂NR⁶R⁷, CONR⁶R⁷, or (CH₂)_{q}NR⁶R⁷, wherein q is an integer from 0 to 5, inclusive;
m is an integer from 0 to 3, inclusive;
or R⁴ₘ is a fused cyclic moiety comprising from 3 to 5 additional ring carbon atoms, inclusive, and optionally comprising at least one heteroatom or heterogroup selected independently from O, N, S, NR¹⁰, SO₂, or CO;
R⁵, in each occurrence, is selected independently from: 1) an alkoxy, a haloalkoxy, or a cycloalkyl, any of which having up to 12 carbon atoms; 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO; 3) hydroxyl, NR⁶R⁷, CO₂R₆, COR⁸, or SO₂R⁸; or 4) a substituted or an unsubstituted heterocycloalkyl comprising from 3 to 7 ring carbon atoms, and from 1 to 3 heteroatoms or heterogroups, inclusive, selected independently from O, N, S, NR¹⁰, SO₂, or CO;
R⁶ and R⁷, in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
or R⁶ and R⁷ together with the nitrogen atom to which they are attached form a substituted or an unsubstituted cyclic moiety having from 3 to 7 ring carbon atoms, and optionally comprising 1, 2, or 3 heteroatoms in addition to the nitrogen atom to which R⁶ and R⁷ are bonded, selected independently from O, N, S, or NR¹⁰;
R⁸, in each occurrence, is selected independently from: 1) an alkyl, a cycloalkyl, or a haloalkyl, any of which having up to 12 carbon atoms; or 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
Z is N or CH; or the ZR¹ moiety is S, CO, or SO₂; or the ZR¹R² moiety is —C≡CR²;
R¹¹ is selected independently from:
1) an alkyl, a haloalkyl, a cycloalkyl, or an alkoxycarbonyl, any of which having up to 12 carbon atoms;
2) a substituted or an unsubstituted heteroaryl or heterocyclyl, any of which having up to 12 carbon atoms, comprises at least one heteroatom or heterogroup selected independently from O, N, S, NR¹⁰, SO₂, or CO, wherein any substituted heteroaryl or heterocyclyl is substituted with up to three substituents selected independently from an alkyl having up to 12 carbon atoms or a hydroxyl; or
3) —CO—Z²—R¹³, —CO—R¹², —CO—Z²—(CH₂)ᵣ—CO—Z²—R¹³, —NR¹⁵R¹⁶, —Z²—CO—(CH₂)ᵣ—Z²—R¹³, —Z²—CO—(CH₂)ᵣ—CO—Z²—R¹³, —O—(CH₂)ᵣ—CO—Z²—R¹³, —O—(CH₂)ᵣ—R¹⁴, —O—R¹²—(CH₂)ᵣ—R¹³, —O—R¹⁴—CO—O—R¹³, —O—(CH₂)ᵣ—R¹², —O—(CH₂)ᵣ—NR′R″, —O—(CH₂)ᵣ—CO₂—(CH₂)ᵣ—R¹³, —O—(CH₂)ᵣ—SR⁸, —O—(CH₂)ᵣ—CO₂—R¹³, —O—(CH₂)ᵣ—CONR′R″, —O—(CH₂)ᵣ—CONH—(CH₂)ᵣ—OR¹³, —O—(CH₂)ᵣ—SO₂R⁸, —O—(CH₂)ᵣ—R¹³, —O—(CH₂)ᵣ—OR¹³, —O—(CH₂)ᵣ—O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″, —(CH₂)ᵣ—O—CO—R⁸, —(CH₂)ᵣ—R¹², —(CH₂)ᵣ—R¹³, —(CH₂)ᵣ—CO—Z²—R¹³, —(CH₂)ᵣ—Z²—R¹³, or -alkenylene-CO₂—(CH₂)ᵣ—R¹³;
r, in each occurrence, is independently 1, 2, or 3;
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl, any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
Z², in each occurrence, is selected independently from NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹², —CO₂—(CH₂)ᵣ—CO—Z²—R¹³, —CO₂—(CH₂)ᵣ—OR¹³, —CO—(CH₂)ᵣ—O—(CH₂)ᵣ—O—(CH₂)ᵣ—R¹³, —CO—(CH₂)ᵣ—O(CH₂)ᵣ—OR¹³, or —CO—NH—(CH₂)ᵣ—OR¹³;
or R¹⁵ and R¹⁶ together with the nitrogen atom to which they are attached form a substituted or an unsubstituted cyclic moiety comprising up to 12 carbon atoms, optionally comprising at least one additional heteroatom or heterogroup selected independently from O, N, S, NR¹⁰, SO₂, or CO; wherein any substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰; or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂R⁸, —SO₂NR′R″, or —NR′R″;
wherein the —(CH₂)ᵣ— linking moiety, in any occurrence, is optionally substituted with at least one group selected independently from hydroxyl, amino, or an alkyl having up to 3 carbon atoms;
when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl, any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
when R¹ and R² together with the diradical Z to which they are attached form a monocyclic or a bicyclic moiety, the cyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl or a heteroaryloxy, any of which having up to 12 carbon atoms, wherein any heteroaryl or heteroaryloxy comprises at least one heteroatom or heterogroup selected independently from O, N, S, or NR¹⁰; or 3) CO₂R⁶, COR⁸, SO₂R⁸, SO₂NR⁶R⁷, or CONR⁶R⁷;
R⁴, R⁶, R⁷, and R⁸ are optionally substituted with at least one substituent, and when substituted, the substituents are selected independently from: 1) halogen, hydroxy, cyano, or NR⁶R⁷; or 2) an alkyl or an alkoxy, any of which having up to 12 carbon atoms;
and R⁵ is optionally substituted with at least one substituent, and when substituted, the substituents are selected independently from: 1) halogen, hydroxy, cyano, or NR⁶R⁷; or 2) an alkyl having up to 12 carbon atoms; and
(ii) the CETP inhibitor having formula (Ia′) is: 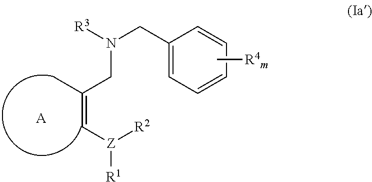
or a stereoisomer thereof or a pharmaceutically acceptable salt thereof, wherein A-ZR¹R² is: 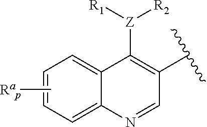
wherein R^{a}, in each occurrence, is selected independently from: 1) a hydrogen, a halogen, a cyano, or a hydroxyl; 2) an alkyl, a haloalkyl, a cycloalkyl, a (cycloalkyl)alkyl, an alkoxy, a cycloalkoxy, a haloalkoxy, an aryl, an aralkyl, a heteroaryl or a heterocyclyl, any of which having up to 12 carbon atoms, wherein any heteroaryl or heterocyclyl, comprises at least one heteroatom or heterogroup selected independently from O, N, S, NR¹⁰, SO₂, or CO; 3) CO₂R⁶, COR⁸, NR⁶R⁷ or SO₂R⁸;
p is an integer from 0 to 3, inclusive;
Z is N or CH; or the ZR¹ moiety is S, SO, CO, or SO₂; or the ZR¹R² moiety is C≡CR² or —C(O)Z³R^{f} wherein R^{f} is an alkyl, a cycloalkyl, or a (cycloalkyl), any of which having up to 12 carbon atoms, or hydrogen; and Z³ is O or NR^{k}, wherein R^{k} is an alkyl, a cycloalkyl, or a (cycloalkyl)alkyl, any of which having up to 12 carbon atoms, or hydrogen;
R¹ and R² are selected independently from: 1) hydrogen; 2) an alkyl having up to 6 carbon atoms; 3) a cycloalkyl having up to 6 carbon atoms; 4) COR⁸; or 5) (CH₂)ₙR⁵ to or (CH₂)ₙR^{d}CO₂R^{e}; wherein n, in each occurrence, is 1 or 2; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, NR¹⁰, SO₂, or CO; and R^{e}, in each occurrence, is selected independently from an alkyl or a cycloalkyl, either of which having up to 12 carbon atoms, or hydrogen;
or R¹ and R² together form a substituted or an unsubstituted monocyclic or bicyclic moiety comprising up to 12 carbon atoms, and optionally comprising 1 or 2 heteroatoms or heterogroups selected independently from O, N, or NR¹⁰; wherein any optional substituent on the cyclic moiety selected from: 1) a cycloalkyl having up to 6 carbon atoms; or 2) an alkyl having up to 2 carbon atoms;
R³ is selected from: 1) cyano; 2) a substituted or an unsubstituted alkyl having up to 12 carbon atoms; 3) a substituted or an unsubstituted aryl, or a substituted or an unsubstituted 5-, 6-, or 7-membered heterocyclyl or heteroaryl, comprising 1, 2, or 3 heteroatoms or heterogroups selected independently from O, N, S, NR¹⁰, SO₂, or CO; any of which having up to 12 carbon atoms; or 4) CO₂R⁶, COR⁸, SO₂R⁸, SO₂NR⁶R⁷, CONR⁶R⁷, C(S)NR⁶R⁷, C(═NH)OR⁸, C(S)NHC(O)OR⁸, or C(S)SR⁸; wherein when R³ is an alkyl, an aryl, a heterocyclyl or a heteroaryl, R³ is optionally substituted with up to three substituents selected independently from R¹¹;
R⁴, in each occurrence, is selected independently from: 1) halogen, hydroxy or cyano; 2) or an alkyl, an alkoxy, a haloalkyl, or a haloalkoxy any of which having up to 4 carbon atoms; and m is an integer from 1-3, inclusive;
R⁵, in each occurrence, is selected independently from: 1) a substituted or an unsubstituted cycloalkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁶ and R⁷, in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁸, in each occurrence, is selected independently from: 1) an alkyl, a cycloalkyl, or a haloalkyl, any of which having up to 12 carbon atoms; or 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
R¹¹ is selected independently from:
1) a halogen, a hydroxyl or a cyano;
2) an alkyl, a haloalkyl, an alkoxy, a cycloalkyl, or an alkoxycarbonyl, any of which having up to 12 carbon atoms:
3) a substituted or an unsubstituted heteroaryl or heterocyclyl, any of which having up to 12 carbon atoms, comprises at least one heteroatom or heterogroup selected independently from O, N, S, NR¹⁰, SO₂, or CO, wherein any substituted heteroaryl or heterocyclyl is substituted with up to three substituents selected independently from an alkyl having up to 12 carbon atoms or a hydroxyl; or
4) —CO—Z²—R¹³, —CO—R¹², —CO—Z²—(CH₂)ᵣ—CO—Z²—R¹³, —NR¹⁵R¹⁶,
—Z²—CO—(CH₂)ᵣ—Z²—R¹³, —Z²—CO—(CH₂)ᵣ—CO—Z²—R¹³, —O—(CH₂)ᵣ—CO—Z²—R¹³,
—O—(CH₂)ᵣ—R¹⁴, —O—R¹²—(CH₂)ᵣ—R¹³, —O—R¹⁴—CO—O—R¹³, —O—(CH₂)ᵣ—R¹²,
—O—(CH₂)ᵣ—NR′R″, —O—(CH₂)ᵣ—CO₂—(CH₂)ᵣ—R¹³, —O—(CH₂)ᵣ—SR⁸, —O—(CH₂)ᵣ—CO₂—R¹³,
—O—(CH₂)ᵣ—O—(CH₂)ᵣ—OR¹³, —O—(CH₂)ᵣ—CONR′R″, —O—(CH₂)ᵣ—CONH—(CH₂)ᵣ—OR¹³, —O—(CH₂)ᵣ—SO₂R⁸,
—O—(CH₂)ᵣ—R¹³, —O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″,
—(CH₂)ᵣ—O—CO—R⁸, —(CH₂)ᵣ—R¹², —(CH₂)ᵣ—R¹³, —(CH₂)ᵣ—NH—(CH₂)ᵣ—OR¹³,
—(CH₂)ᵣ—CO—Z²—R¹³, —(CH₂)ᵣ—Z²—R¹³, —(CH₂)ᵣ—NH—CO—Z²—R¹³, or -alkenylene-CO₂—(CH₂)ᵣ—R¹³;
r, in each occurrence, is independently 1, 2 or 3;
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
Z², in each occurrence, is selected independently from NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹², —CO₂—(CH₂)ᵣ—CO—Z²—R¹³, —CO₂—(CH₂)ᵣ—OR¹³, —CO—(CH₂)ᵣ—O—(CH₂)ᵣ—O—(CH₂)ᵣ—R¹³, —CO—(CH₂)ᵣ—O(CH₂)ᵣ—OR¹³, or —CO—NH—(CH₂)ᵣ—OR¹³;
or R¹⁵ and R¹⁶ together form a substituted or an unsubstituted cyclic moiety comprising up to 12 carbon atoms, optionally comprising at least one additional heteroatom or heterogroup selected independently from O, N, S, NR¹⁰, SO₂, or CO; wherein any substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰; or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂R⁸, —SO₂NR′R″, or —NR′R″; and
wherein the —(CH₂)ᵣ— linking moiety, in any occurrence, is optionally substituted with at least one group selected independently from hydroxyl, amino, or an alkyl having up to 3 carbon, atoms;
wherein when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl, any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
wherein when R¹ and R² together form a monocyclic or a bicyclic moiety, the monocyclic or bicyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl or a heteroaryloxy, any of which having up to 12 carbon atoms, wherein any heteroaryl or heteroaryloxy comprises at least one heteroatom or heterogroup selected independently from O, N, S, or NR¹⁰; or 3) CO₂R⁶, (CH₂)_{q}COR⁸, SO₂R⁸, SO₂NR⁶R⁷, or CONR⁶R⁷; or 4) (CH₂)_{q}CO₂(CH₂)_{q}CH₃, wherein q is selected independently from an integer from 0 to 3, inclusive; and
R⁴, R⁶, R⁷, and R⁸ are optionally substituted with at least one substituent, and when substituted, the substituents are selected independently from: 1) halogen, hydroxy, cyano, or NR⁶R⁷; or 2) an alkyl or an alkoxy, any of which having up to 12 carbon atoms; and
R⁵ is optionally substituted with at least one substituent selected independently from: 1) halogen, hydroxy, cyano, or NR⁶R⁷; or 2) an alkyl or an alkoxy, any of which having up to 12 carbon atoms; or 3) (CH₂)ₜOR^{j} or (CH₂)ₜCOOR^{j} wherein t is an integer from 1 to 3, inclusive, and R^{j} is hydrogen or alkyl having up to 12 carbon atoms;
(iii) the CETP inhibitor having formula (II) is: 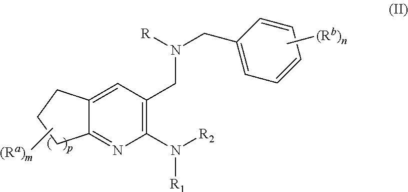
or a stereoisomer thereof or a pharmaceutically acceptable salt thereof; wherein,
R represents 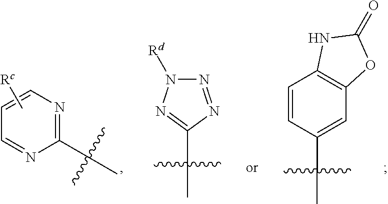
R¹ and R² are independently selected from hydrogen, acyl, haloalkyl, —(CHR^{e})_{q}R³, an optionally substituted group selected from alkyl or cycloalkyl, wherein optional substituent, in each occurrence, is independently selected from halogen, cyano, hydroxyl, an alkyl, a haloalkyl or an alkoxy;
R³ is a group selected from alkoxy, haloalkoxy, cycloalkyl, aryl, heterocyclyl or heteroaryl, wherein R³ is optionally substituted with a group selected from halogen, cyano, hydroxyl, alkyl, haloalkyl or alkoxy;
R^{a}, in each occurrence, is independently selected from halogen, cyano, hydroxy, alkyl, haloalkyl or alkoxy;
R^{b}, in each occurrence, is independently selected from halogen, alkyl, haloalkyl, hydroxy, alkoxy, or haloalkoxy;
R^{c} is independently selected from hydrogen, cyano, halogen, —C(═O)—R^{f}, —CONR^{g}R^{h}, —C(═O)—CH≡CH—NRⁱR^{j}, an optionally substituted group selected from cycloalkyl, aryl, heteroaryl or heterocyclyl ring, wherein the optional substituent, in each occurrence, is selected independently from hydrogen, halogen, cyano, hydroxyl, alkyl, haloalkyl, alkoxy, alkoxyalkyl or haloalkoxy;
R^{d} is hydrogen or alkyl;
R^{e}, in each occurrence, is independently selected from hydrogen, alkyl or alkoxy;
R^{f} is hydrogen or alkyl;
R^{g} and R^{h} independently represent hydrogen or alkyl;
Rⁱ and R^{j} independently represent hydrogen or alkyl;
m is 0, 1 or 2;
n is 0, 1, 2 or 3;
p is 1 or 2; and
q is 0, 1, 2, 3, 4 or 5; and
(iv) the CETP inhibitor having formula (III) is: 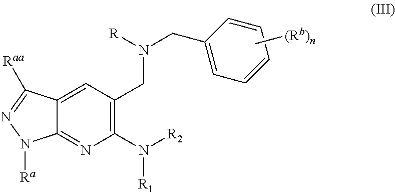
or a stereoisomer thereof or a pharmaceutically acceptable salt thereof; wherein,
R represents hydrogen or 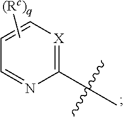
X represents —CH or —N;
R¹ and R² are independently of each other selected from hydrogen, acyl, alkyl or —(CH₂)ₚ-cycloalkyl;
R^{a} and R^{aa} are independently of each other selected from hydrogen or alkyl;
R^{b}, in each occurrence, is independently selected from halogen, alkyl, haloalkyl, hydroxy, alkoxy or haloalkoxy;
R^{c}, in each occurrence, is independently selected from hydrogen, cyano, halogen, alkyl, alkoxy, haloalkoxy, —COOR^{d}, —C(═O)—R^{e}, —CONR^{g}R^{h}, —C(═O)—CH═CH—NRⁱR^{j}, —NHCOR^{t}, an optionally substituted group selected from cycloalkyl, a heteroaryl or heterocycle ring, wherein the optional substituent, in each occurrence, is selected independently from hydrogen, halogen, cyano, hydroxyl, alkyl, haloalkyl, alkoxy, alkoxyalkyl or haloalkoxy;
R^{d}, R^{e}, R^{g}, R^{h}, Rⁱ and R^{j}, in each occurrence, independently of each other represents hydrogen or alkyl;
R^{t} is selected from hydrogen, alkyl or cycloalkyl;
n is 0, 1, 2 or 3;
p is 0, 1, or 2; and
q is 1 or 2.

13. (canceled)

14. The composition according to claim 9, wherein said CETP inhibitor is at least 10% in amorphous state in said composition.

15. The composition according to claim 1, wherein said composition releases not more than 50% at a period of 30 minutes in 900 ml of simplified simulated intestinal fluid having a pH of 6.5, when tested in a USP Type 2 apparatus at 25 rpm and 37° C.

16. The composition according to claim 11, wherein said composition releases not more than 75% at a period of 60 minutes and not less than 90% at a period of 360 minutes.

17. (canceled)

18. The composition according to claim 9, wherein the dispersion is sprayed on to an inert carrier in a liquid state to form a solid amorphous dispersion, wherein at least 10 wt % of said CETP inhibitor being noncrystalline, wherein said CETP inhibitor has a solubility in aqueous solution in the absence of said solubility improving material of less than 2 μg/ml at any pH of from 1 to 8.

19. The composition according to claim 9, wherein the composition is prepared a process comprising:
a) dissolving a CETP inhibitor having formula (I) or (Ia′) or (II) or (III) and at least one solubility improving material in one or more solvents,
b) optionally adding one or more wetting agents to the mixture of step a,
c) spray-drying the mixture of step b, to remove the solvent and to form a solid amorphous dispersion,
d) collecting the spray-dried solid amorphous dispersion powder, and
e) combining the solid amorphous dispersion powder of step d, with at least one pharmaceutically acceptable excipient to form desired dosage form.

20. The composition according to claim 9, wherein the composition is prepared a process comprising:
a) dissolving a CETP inhibitor having formula (I) or (Ia′) or (II) or (III) and at least one solubility improving material in one or more solvents,
b) optionally adding one or more wetting agents to the mixture of step a,
c) spraying the mixture of step b over inert carrier,
d) collecting the solid amorphous dispersion layered carrier, and
e) combining the solid amorphous dispersion layered carrier of step d, with at least one pharmaceutically acceptable excipient to form desired dosage form.

21.-22. (canceled)

23. A composition comprising a CETP inhibitor of formula (I), (Ia′), (II) or (III) and at least one solubility improving material, wherein said composition when administered to a mammal provides the area under the curve (AUC₀₋₄₈) profile in fed to fast state in a ratio of about 1 to 3 and the maximum plasma profile (Cₘₐₓ) in fed to fast state in a ratio of about 1 to 3, wherein
(a) the CETP inhibitor having formula (I) is: 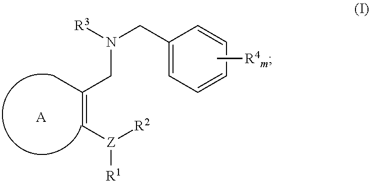
or a stereoisomer thereof or a pharmaceutically acceptable salt thereof wherein:
A is a substituted or an unsubstituted quinoline moiety having the formula: 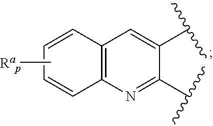
wherein R^{a}, in each occurrence, is selected independently from: 1) a halogen; a hydroxyl, or a cyano; 2) an alkyl or an alkoxy, any of which having up to 12 carbon atoms; or 3) CO₂R⁶; and p is an integer from 0 to 3, inclusive;
R¹ and R² are selected independently from: 1) hydrogen; 2) a substituted or an unsubstituted cycloalkyl, haloalkyl, aryl, heterocyclyl, heteroaryl, any of which having up to 12 carbon atoms, wherein any heterocyclyl or heteroaryl comprises at least one heteroatom or heterogroup selected independently from O, N, S, NR¹⁰, SO₂, or CO; 3) CO₂R⁶, COR⁸, SO₂R⁸, SO₂NR⁶R⁷, or CONR⁶R⁷; or 4) (CHR^{x})ₙR⁵ or (CH₂)ₙR^{d}CO₂R^{e}, wherein n, in each occurrence, is 1, 2, or 3; R^{x}, in each occurrence, is selected independently from an alkyl or an alkoxy, either of which having up to 12 carbon atoms, or hydrogen; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, NR¹⁰, SO₂ or CO; and R^{e}, in each occurrence, is selected independently from an alkyl or a cycloalkyl, either of which having up to 12 carbon atoms or hydrogen;
or R¹ and R² together with the diradical Z to which they are attached form a substituted or an unsubstituted monocylic or bicyclic moiety comprising up to 12 carbon atoms, and optionally comprising 1, 2, or 3 heteroatoms or heterogroups in addition to Z, selected independently from O, N, S, NR¹⁰, SO₂, or CO;
R³ is selected from: 1) hydrogen or cyano; 2) a substituted alkyl having up to 12 carbon atoms; 3) a substituted or an unsubstituted aryl, or a substituted or an unsubstituted 5-, 6-, or 7-membered heterocyclyl or heteroaryl, any of which having up to 12 carbon atoms, comprising 1, 2, or 3 heteroatoms or heterogroups selected independently front O, N, S, NR¹⁰, SO₂, or CO; or 4) CO₂R⁶, COR⁸, SO₂R⁸, SO₂NR⁶R⁷, CONR⁶R⁷, C(S)NR⁶R⁷, C(S)NC(O)OR⁸, or C(S)SR⁸; or 5) a substituted or an unsubstituted group selected from 4,5-dihydro-oxazolyl, tetrazolyl, isoxazolyl, pyridyl, pyrimidinyl, oxadiazolyl, thiazolyl, or oxazolyl; wherein any optional substituent is selected independently from: a) an alkyl or haloalkyl, any of which having up to 12 carbon atoms; or b) CO₂R⁹, wherein R⁹ is an alkyl having up to 12 carbon atoms;
wherein when R³ is an aryl, a heterocyclyl, or a heteroaryl, R³ is optionally substituted with up to three substituents selected independently from a halogen, a hydroxyl, a cyano, an alkoxy having up to 12 carbon atoms, or R¹¹;
R⁴, in each occurrence, is selected independently from: 1) halogen, cyano, or hydroxy; 2) an alkyl, a cycloalkyl, a cycloalkoxy, an alkoxy, a haloalkyl, or a haloalkoxy, any of which having up to 12 carbon atoms; 3) a substituted or an unsubstituted aryl, aralkyl, aryloxy, heteroaryl, or heteroaryloxy, any of which having up to 12 carbon atoms, wherein any heteroaryl or heteroaryloxy comprises at least one heteroatom or heterogroup selected independently from O, N, S, or NR¹⁰; or 4) CO₂R⁶, COR⁸, SO₂R⁸, SO₂NR⁶R⁷, CONR⁶R⁷, or (CH₂)_{q}NR⁶R⁷, wherein q is an integer from 0 to 5, inclusive;
m is an integer from 0 to 3, inclusive;
or R⁴ₘ is a fused cyclic moiety comprising from 3 to 5 additional ring carbon atoms, inclusive, and optionally comprising at least one heteroatom or heterogroup selected independently from O, N, S, NR¹⁰, SO₂, or CO;
R⁵, in each occurrence, is selected independently from: 1) an alkoxy, a haloalkoxy, or a cycloalkyl, any of which having up to 12 carbon atoms; 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO; 3) hydroxyl, NR⁶R⁷, CO₂R⁶, COR⁸, or SO₂R⁸; or 4) a substituted or an unsubstituted heterocycloalkyl comprising from 3 to 7 ring carbon atoms, and from 1 to 3 heteroatoms or heterogroups, inclusive, selected independently from O, N, S, NR¹⁰, SO₂, or CO;
R⁶ and R⁷, in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
or R⁶ and R⁷ together with the nitrogen atom to which they are attached form a substituted or an unsubstituted cyclic moiety having from 3 to 7 ring carbon atoms, and optionally comprising 1, 2, or 3 heteroatoms in addition to the nitrogen atom to which R⁶ and R⁷ are bonded, selected independently from O, N, S, or NR¹⁰;
R⁸, in each occurrence, is selected independently from: 1) an alkyl, a cycloalkyl, or a haloalkyl, any of which having up to 12 carbon atoms; or 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
Z is N or CH; or the ZR¹ moiety is S, CO, or SO₂; or the ZR¹R² moiety is —C≡CR²;
R¹¹ is selected independently from:
1) an alkyl, a haloalkyl, a cycloalkyl, or an alkoxycarbonyl, any of which having up to 12 carbon atoms;
2) a substituted or an unsubstituted heteroaryl or heterocyclyl, any of which having up to 12 carbon atoms, comprises at least one heteroatom or heterogroup selected independently from O, N, S, NR¹⁰, SO₂, or CO, wherein any substituted heteroaryl or heterocyclyl is substituted with up to three substituents selected independently from an alkyl having up to 12 carbon atoms or a hydroxyl; or
3) —CO—Z²—R¹³, —CO—R¹², —CO—Z²—(CH₂)ᵣ—CO—Z²—R¹³, —NR¹⁵R¹⁶, —Z²—CO—(CH₂)ᵣ—Z²—R¹³, —Z²—CO—(CH₂)ᵣ—CO—Z²—R¹³, —O—(CH₂)ᵣ—CO—Z²—R¹³, —O—(CH₂)ᵣ—R¹⁴, —O—R¹²—(CH₂)ᵣ—R¹³, —O—R¹⁴—CO—O—R¹³, —O—(CH₂)ᵣ—R¹², —O—(CH₂)ᵣ—NR′R″, —O—(CH₂)ᵣ—CO₂—(CH₂)ᵣ—R¹³, —O—(CH₂)ᵣ—SR⁸, —O—(CH₂)ᵣ—CO₂—R¹³, —O—(CH₂)ᵣ—CONR′R″, —O—(CH₂)ᵣ—CONH—(CH₂)ᵣ—OR¹³, —O—(CH₂)ᵣ—SO₂R⁸, —O—(CH₂)ᵣ—R¹³, —O—(CH₂)ᵣ—OR¹³, —O—(CH₂)ᵣ—O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″, —(CH₂)ᵣ—O—CO—R⁸, —(CH₂)ᵣ—R¹², —(CH₂)ᵣ—R¹³, —(CH₂)ᵣ—CO—Z²—R¹³, —(CH₂)ᵣ—Z²R¹³, or -alkenylene-CO₂—(CH₂)ᵣ—R¹³;
r, in each occurrence, is independently 1, 2, or 3;
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl, any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
Z², in each occurrence, is selected independently from NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹³, —CO—(CH₂)ᵣ—CO—Z²—R¹³, —CO₂—(CH₂)ᵣ—OR¹³, —CO—(CH₂)ᵣ—O—(CH₂)ᵣ—O—(CH₂)ᵣ—R¹³, —CO—(CH₂)ᵣ—O(CH₂)ᵣ—OR¹³, or —CO—NH—(CH₂)ᵣ—OR¹³;
or R¹⁵ and R¹⁶ together with the nitrogen atom to which they are attached form a substituted or an unsubstituted cyclic moiety comprising up to 12 carbon atoms, optionally comprising at least one additional heteroatom or heterogroup selected independently from O, N, S, NR¹⁰, SO₂, or CO; wherein any substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰; or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂R⁸, —SO₂NR′R″, or —NR′R″;
wherein the —(CH₂)ᵣ— linking moiety, in any occurrence, is optionally substituted with at least one group selected independently from hydroxyl, amino, or an alkyl having up to 3 carbon atoms;
when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl, any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
when R¹ and R² together with the diradical Z to which they are attached form a monocyclic or a bicyclic moiety, the cyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl or a heteroaryloxy, any of which having up to 12 carbon atoms, wherein any heteroaryl or heteroaryloxy comprises at least one heteroatom or heterogroup selected independently from O, N, S, or NR¹⁰; or 3) CO₂R⁶, COR⁸, SO₂R⁸, SO₂NR⁶R⁷, or CONR⁶R⁷;
R⁴, R⁶, R⁷, and R⁸ are optionally substituted with at least one substituent, and when substituted, the substituents are selected independently from: 1) halogen, hydroxy, cyano, or NR⁶R⁷; or 2) an alkyl or an alkoxy, any of which having up to 12 carbon atoms;
and R⁵ is optionally substituted with at least one substituent, and when substituted, the substituents are selected independently from: 1) halogen, hydroxy, cyano, or —NR⁶R⁷; or 2) an alkyl having up to 12 carbon atoms; and
(ii) the CETP inhibitor having formula (Ia′) is: 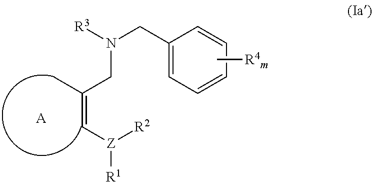
or a stereoisomer thereof or a pharmaceutically acceptable salt thereof, wherein A-ZR¹R² is: 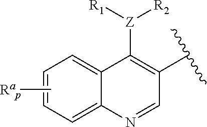
wherein R^{a}, in each occurrence, is selected independently from: 1) a hydrogen, a halogen, a cyano, or a hydroxyl; 2) an alkyl, a haloalkyl, a cycloalkyl, a (cycloalkyl)alkyl, an alkoxy, a cycloalkoxy, a haloalkoxy, an aryl, an aralkyl, a heteroaryl or a heterocyclyl, any of which having up to 12 carbon atoms, wherein any heteroaryl or heterocyclyl, comprises at least one heteroatom or heterogroup selected independently from O, N, S, NR¹⁰, SO₂, or CO; 3) CO₂R⁶, COR⁸, NR⁶R⁷ or SO₂R⁸;
p is an integer from 0 to 3, inclusive;
Z is N or CH; or the ZR¹ moiety is S, SO, CO, or SO₂; or the ZR¹R² moiety is C≡CR² or —C(O)Z³R^{f}, wherein R^{f} is an alkyl, a cycloalkyl, or a (cycloalkyl)alkyl, any of which having up to 12 carbon atoms, or hydrogen; and Z³ is O or NR^{k}, wherein R^{k} is an alkyl, a cycloalkyl, or a (cycloalkyl)alkyl, any of which having up to 12 carbon atoms, or hydrogen;
R¹ and R² are selected independently from: 1) hydrogen; 2) an alkyl having up to 6 carbon atoms; 3) a cycloalkyl having up to 6 carbon atoms; 4) COR⁸; or 5) (CH₂)ₙR⁵ or (CH₂)ₙR^{d}CO₂R^{e}; wherein n, in each occurrence, is 1 or 2; R^{d}, in each occurrence, is selected independently from an alkyl, a cycloalkyl, an aryl, a heterocyclyl, or a heteroaryl, any of which having up to 12 carbon atoms, wherein any heterocyclyl or heteroaryl comprises at least one heteroatom or heterogroup selected independently from O, N, S, NR¹⁰, SO₂, or CO; and R^{e}, in each occurrence, is selected independently from an alkyl or a cycloalkyl, either of which having up to 12 carbon atoms, or hydrogen;
or R¹ and R² together form a substituted or an unsubstituted monocyclic or bicyclic moiety comprising up to 12 carbon atoms, and optionally comprising 1 or 2 heteroatoms or heterogroups selected independently from O, N, or NR¹⁰; wherein any optional substituent on the cyclic moiety selected from: 1) a cycloalkyl having up to 6 carbon atoms; or 2) an alkyl having up to 2 carbon atoms;
R³ is selected from: 1) cyano; 2) a substituted or an unsubstituted alkyl having up to 12 carbon atoms; 3) a substituted or an unsubstituted aryl, or a substituted or an unsubstituted 5-, 6-, or 7-membered heterocyclyl or heteroaryl, comprising 1, 2, or 3 heteroatoms or heterogroups selected independently from O, N, S, NR¹⁰, SO₂, or CO; any of which having up to 12 carbon atoms; or 4) CO₂R⁶, COR⁸, SO₂R⁸, SO₂NR⁶R⁷, CONR⁶R⁷, C(S)NR⁶R⁷, C(═NH)OR⁸, C(S)NHC(O)OR⁸, or C(S)SR⁸; wherein when R³ is an alkyl, an aryl, a heterocyclyl or a heteroaryl, R³ is optionally substituted with up to three substituents selected independently from R¹¹;
R⁴, in each occurrence, is selected independently from: 1) halogen, hydroxy or cyano; or 2) an alkyl, an alkoxy, a haloalkyl, or a haloalkoxy any of which having up to 4 carbon atoms; and m is an integer from 1-3, inclusive;
R⁵, in each occurrence, is selected independently from: 1) a substituted or an unsubstituted cycloalkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, CO;
R⁶ and R⁷, in each occurrence, are selected independently from: 1) hydrogen; 2) an alkyl, a cycloalkyl, or a haloalkyl, any of which having up to 12 carbon atoms; or 3) a substituted or an unsubstituted aryl, aralkyl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R⁸, in each occurrence, is selected independently from: 1) an alkyl, a cycloalkyl, or a haloalkyl, any of which having up to 12 carbon atoms; or 2) a substituted or an unsubstituted aryl, heterocyclyl, or heteroaryl, any of which having up to 12 carbon atoms, wherein any heterocyclyl or heteroaryl comprises at least one heteroatom or heterogroup selected independently from O, N, S, NR¹⁰, SO₂, or CO;
R¹⁰, in each occurrence, is selected independently from: 1) hydrogen; or 2) an alkyl, a cycloalkyl, a haloalkyl, an aryl, or an aralkyl, any of which having up to 12 carbon atoms;
R¹¹ is selected independently from:
1) a halogen, a hydroxyl or a cyano;
2) an alkyl, a haloalkyl, an alkoxy, a cycloalkyl, or an alkoxycarbonyl, any of which having up to 12 carbon atoms;
3) a substituted or an unsubstituted heteroaryl, or heterocyclyl, any of which having up to 12 carbon atoms, comprises at least one heteroatom or heterogroup selected independently from O, N, S, NR¹⁰, SO₂, or CO, wherein any substituted heteroaryl or heterocyclyl is substituted with up to three substituents selected independently from an alkyl having up to 12 carbon atoms or a hydroxyl; or
4) —CO—Z²—R¹³, —CO—R¹², —CO—Z²—(CH₂)ᵣ—CO—Z²—R¹³, —NR¹⁵R¹⁶, —Z²—CO—(CH₂)ᵣ—Z²—R¹³, —Z²—CO—(CH₂)ᵣ—CO—Z²—R¹³, —O—(CH₂)ᵣ—CO—Z²—R¹³, —O—(CH₂)ᵣ—R¹⁴, —O—R¹²—(CH₂)^{r}—R¹³, —O—R¹⁴—CO—O—R¹³, —O—(CH₂)ᵣ—R¹², —O—(CH₂)ᵣ—NR′R″, —O—(CH₂)ᵣ—CO₂—(CH₂)ᵣR¹³, —O—(CH₂)ᵣ—SR⁸, —O—(CH₂)ᵣ—CO₂—R¹³, —O—(CH₂)ᵣ—O—(CH₂)ᵣ—OR¹³, —O—(CH₂)ᵣ—R¹³, —O—(CH₂)ᵣ—OR¹³, —S—(CH₂)ᵣ—CONR′R″, —SO₂—(CH₂)ᵣ—OR¹³, —SO₂—(CH₂)ᵣ—CONR′R″, —(CH₂)ᵣ—O—CO—R⁸, —(CH₂)ᵣ—R¹², —(CH₂)ᵣ—R¹³, —(CH₂)ᵣ—NH—(CH₂)ᵣ—OR¹³, —(CH₂)ᵣ—CO—Z²—R¹³, —(CH₂)ᵣ—Z²—R¹³, —(CH₂)ᵣ—NH—CO—Z²—R¹³, or -alkenylene-CO₂—(CH₂)ᵣ—R¹³;
r, in each occurrence, is independently 1, 2, or 3;
R¹², in each occurrence, is independently selected from a substituted or an unsubstituted heterocyclyl having up to 12 carbon atoms, comprising at least one heteroatom or heterogroup selected independently from O, N, S, NR¹⁰, SO₂, or CO, wherein any substituted heterocyclyl is substituted with up to three substituents selected independently from an acyl, an alkyl, or an alkoxycarbonyl, any of which having up to 12 carbon atoms, or —COOH;
R¹³, in each occurrence, is independently selected from: 1) hydrogen; or 2) a cycloalkyl, an aryl, a haloalkyl, a heterocyclyl, or an alkyl group optionally substituted with at least one hydroxyl, any of which having up to 12 carbon atoms, wherein any heterocyclyl comprises at least one heteroatom or heterogroup selected independently from O, N, S, NR¹⁰, SO₂, or CO;
R¹⁴, in each occurrence, is independently selected from a heterocyclyl, a cycloalkyl, or an aryl, any of which having up to 12 carbon atoms, wherein any heterocyclyl comprises at least one heteroatom or heterogroup selected independently from O, N, S, NR¹⁰, SO₂, or CO;
Z², in each occurrence, is selected independently from —NR¹⁰ or O;
R′ and R″, in each occurrence, are independently selected from hydrogen or an alkyl having up to 12 carbon atoms; and
R¹⁵ and R¹⁶, in each occurrence, are independently selected from: 1) hydrogen; 2) an alkyl having up to 12 carbon atoms; or 3) —(CH₂)ᵣ—O—R¹³, —(CH₂)ᵣ—R¹⁴, —COR¹³, —(CH₂)ᵣ—CO—Z²—R¹³, —CO₂R¹³, —CO₂—(CH₂)ᵣ—R¹³, —CO₂—(CH₂)ᵣ—R¹², —CO₂—(CH₂)ᵣ—CO—Z²—R¹³, —CO₂—(CH₂)ᵣ—OR¹³, —CO—(CH₂)ᵣ—O—(CH₂)ᵣ—O—(CH₂)ᵣ—R¹³, —CO—(CH₂)ᵣ—O(CH₂)ᵣ—OR¹³, or —CO—NH—(CH₂)ᵣ—OR¹³;
or R¹⁵ and R¹⁶ together form a substituted or an unsubstituted cyclic moiety comprising up to 12 carbon atoms, optionally comprising at least one additional heteroatom or heterogroup selected independently from O, N, S, NR¹⁰, SO₂, or CO; wherein an substituted cyclic moiety is substituted with up to three substituents selected independently from: 1) hydroxyl; 2) an alkyl or a heteroaryl, any of which having up to 12 carbon atoms, wherein any heteroaryl comprises at least one heteroatom or heterogroup selected independently from O, N, S, or NR¹⁰; or 3) COOR¹³, —Z²—(CH₂)ᵣ—R¹³, —COR¹³, —CO₂—(CH₂)ᵣ—R¹³, —CO(CH₂)ᵣ—O—R¹³, —(CH₂)ᵣ—CO₂—R¹³, —SO₂R⁸, —SO₂NR′R″, or —NR′R″; and
wherein the —(CH₂)ᵣ— linking moiety, in any occurrence, is optionally substituted with at least one group selected independently from hydroxyl, amino, or an alkyl having up to 3 carbon atoms;
wherein when R¹ and R² do not form a monocyclic or bicyclic moiety, R¹ and R² are optionally substituted with 1 or 2 substituents, and when substituted, the substituents are selected independently from: 1) an alkyl, a cycloalkyl, a haloalkyl, an alkoxy, an aryl, a heteroaryl, or a heterocyclyl any of which having up to 12 carbon atoms, wherein any heteroaryl or heterocyclyl comprises at least one heteroatom or heterogroup selected independently from O, N, S, NR¹⁰, SO₂, or CO; or 2) halogen, cyano, or hydroxyl;
wherein when R¹ and R² together form a monocyclic or a bicyclic moiety, the monocyclic or bicyclic moiety is optionally substituted with at least one substituent selected independently from: 1) halogen, cyano, or hydroxyl; 2) an alkyl, a haloalkyl, a cycloalkyl, an alkoxy, a cycloalkyl-substituted alkyl, an alkoxyalkyl, a cycloalkoxy, a haloalkoxy, an aryl, an aryloxy, an aralkyl, a heteroaryl or a heteroaryloxy, any of which having up to 12 carbon atoms, wherein any heteroaryl or heteroaryloxy comprises at least one heteroatom or heterogroup selected independently from O, N, S, or NR¹⁰; or 3) CO₂R⁶, (CH₂)_{q}COR⁸, SO₂R⁸, SO₂NR⁶R⁷, or CONR⁶R⁷; or 4) (CH₂)_{q}CO₂(CH₂)_{q}CH₃, wherein q is selected independently from an integer from 0 to 3, inclusive; and
R⁴, R⁶, R⁷, and R⁸ are optionally substituted with at least one substituent, and when substituted, the substituents are selected independently from: 1) halogen, hydroxy, cyano, or NR⁶R⁷; or 2) an alkyl or an alkoxy, any of which having up to 12 carbon atoms; and
R⁵ is optionally substituted with at least one substituent selected independently from: 1) halogen, hydroxy, cyano, or NR⁶R⁷; or 2) an alkyl or an alkoxy, any of which having up to 12 carbon atoms; or 3) (CH₂)ₜOR^{j} or (CH₂)ₜCOOR^{j} wherein t is an integer from 1 to 3, inclusive, and R^{j} is hydrogen or alkyl having up to 12 carbon atoms;
(iii) the CETP inhibitor having formula (II) is: 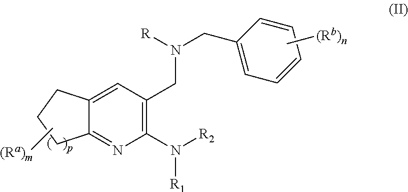
or a stereoisomer thereof or a pharmaceutically acceptable salt thereof; wherein,
R represents 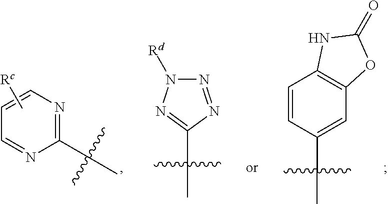
R¹ and R² are independently selected from hydrogen, acyl, haloalkyl, —(CHR^{e})_{q}R³, an optionally substituted group selected from alkyl or cycloalkyl, wherein optional substituent, in each occurrence, is independently selected from halogen, cyano, hydroxyl, an alkyl, a haloalkyl or an alkoxy;
R³ is a group selected from alkoxy, haloalkoxy, cycloalkyl, aryl, heterocyclyl or heteroaryl, wherein R³ is optionally substituted with a group selected from halogen, cyano, hydroxyl, alkyl, haloalkyl or alkoxy;
R^{a}, in each occurrence, is independently selected from halogen, cyano, hydroxy, alkyl, haloalkyl or alkoxy;
R^{b}, in each occurrence, is independently selected from halogen, alkyl, haloalkyl, hydroxy, alkoxy or haloalkoxy;
R^{c} is independently selected from hydrogen, cyano, halogen, —C(═O)—R^{f}, —CONR^{g}R^{h}, —C(═O)—CH≡CH—NRⁱR^{j}, an optionally substituted group selected from cycloalkyl, aryl, heteroaryl, or heterocyclyl ring, wherein the optional substituent, in each occurrence, is selected independently from hydrogen, halogen, cyano, hydroxyl, alkyl, haloalkyl, alkoxy, alkoxyalkyl or haloalkoxy;
R^{d} is hydrogen or alkyl;
R^{e}, in each occurrence, is independently selected from hydrogen, alkyl or alkoxy;
R^{f} is hydrogen or alkyl;
R^{g} and R^{h} independently represent hydrogen or alkyl;
Rⁱ and R^{j} independently represent hydrogen or alkyl;
m in is 0, 1 or 2;
n is 0, 1, 2 or 3;
p is 1 or 2; and
q is 0, 1, 2, 3, 4 or 5; and
(iv) the CETP inhibitor having formula (III) is: 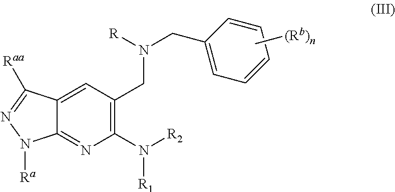
or a stereoisomer thereof or a pharmaceutically acceptable salt thereof; wherein,
R represents hydrogen or 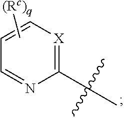
X represents —CH or —N;
R¹ and R² are independently of each other selected from hydrogen, acyl, alkyl or —(CH₂)ₚ-cycloalkyl;
R^{a} and R^{aa} are independently of each other selected from hydrogen or alkyl;
R^{b}, in each occurrence, is independently selected from halogen, alkyl, haloalkyl, hydroxy, alkoxy or haloalkoxy;
R^{c}, in each occurrence, is independently selected from hydrogen, cyano, halogen, alkyl, alkoxy, haloalkoxy, —COOR^{d}, —C(═O)—R^{e}, —CONR^{g}R^{h}, —C(═O)—CH═CH—NRⁱR^{j}, —NHCOR^{t}, an optionally substituted group selected from cycloalkyl, aryl, heteroaryl or heterocycle ring, wherein the optional substituent, in each occurrence, is selected independently from hydrogen, halogen, cyano, hydroxyl, alkyl, haloalkyl, alkoxy, alkoxyalkyl or haloalkoxy;
R^{d}, R^{e}, R^{g}, R^{h}, Rⁱ and R^{j}, in each occurrence, independently of each other represents hydrogen or alkyl;
R^{t} is selected from hydrogen, alkyl or cycloalkyl;
n is 0, 1, 2 or 3;
p is 0, 1, or 2; and
q is 1 or 2.

24.-26.

27. The composition according to claim 1, wherein the CETP inhibitor is selected from a group consisting of: 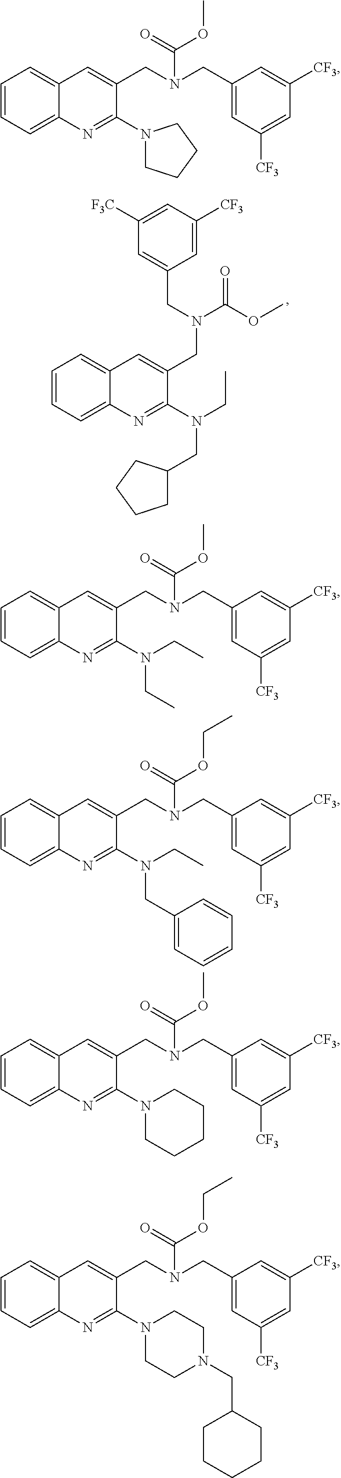 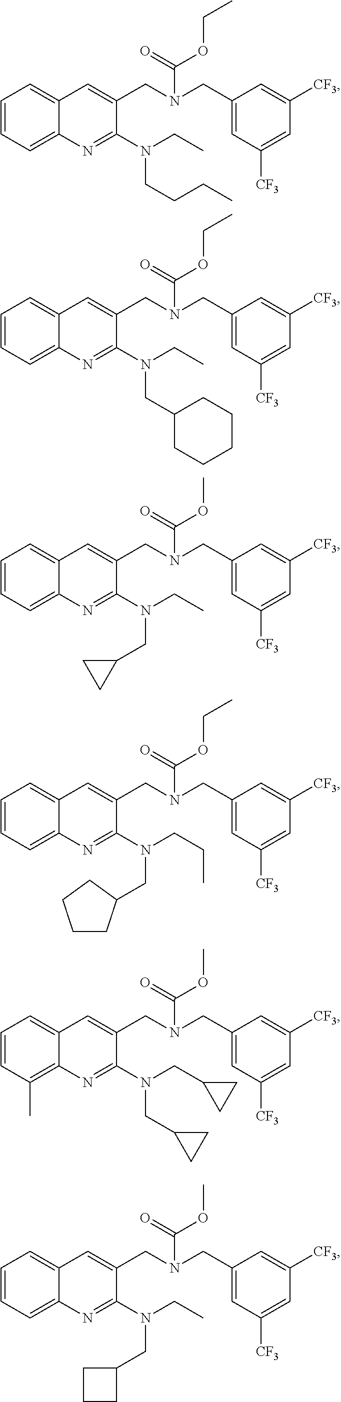 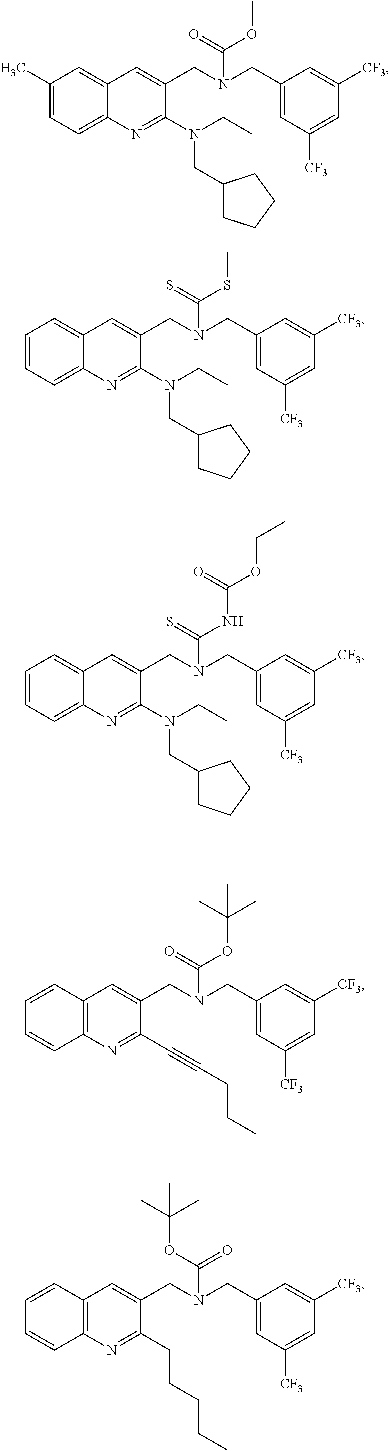 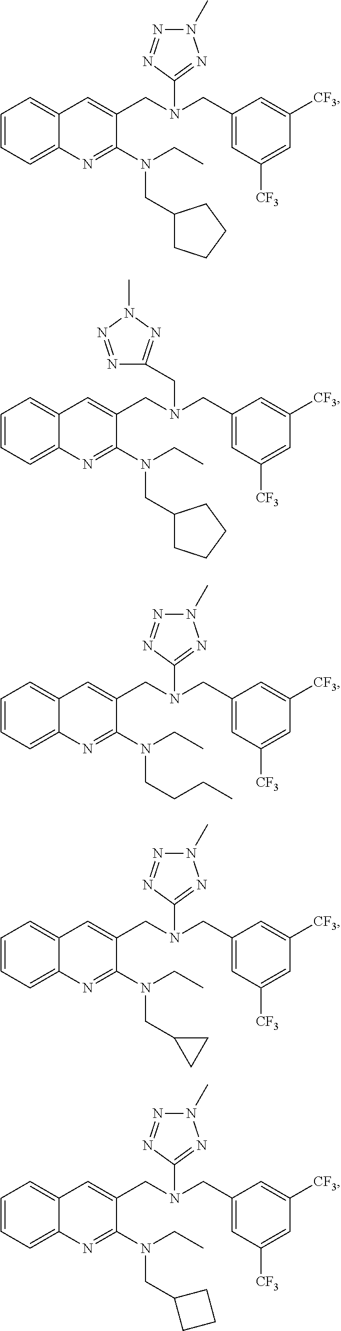 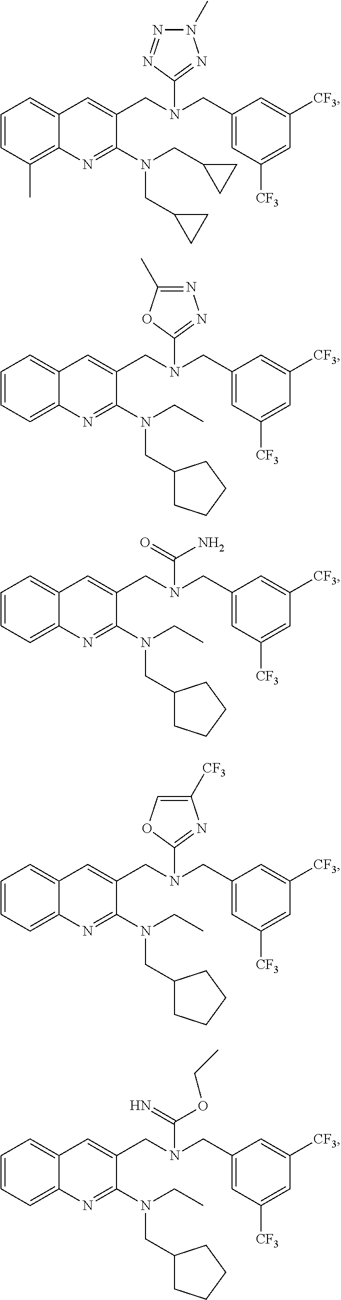 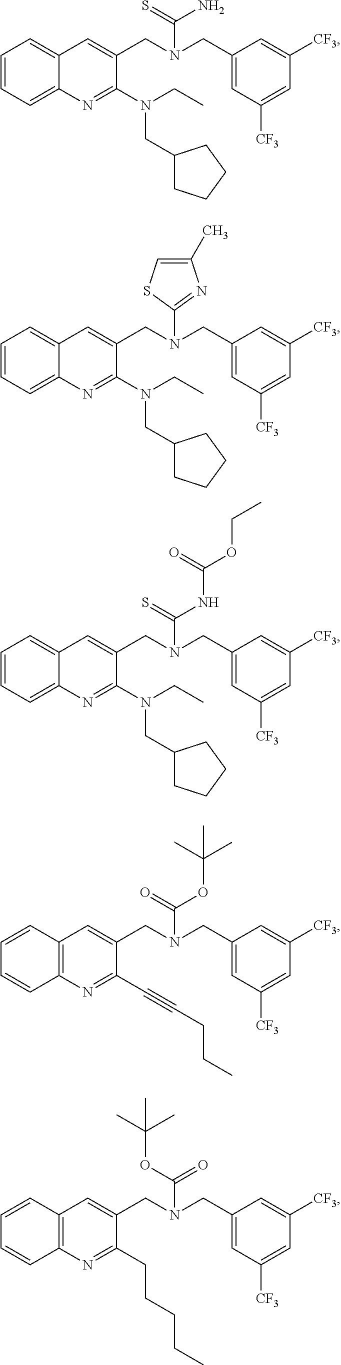 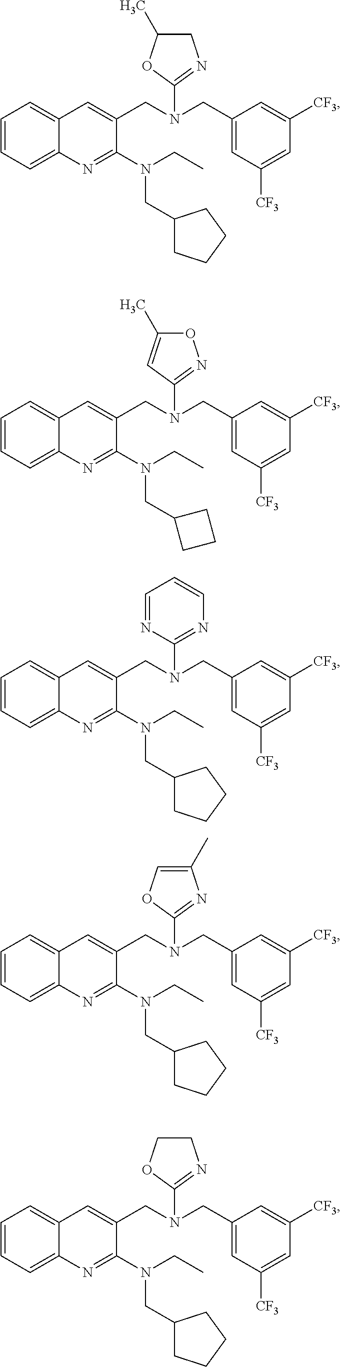 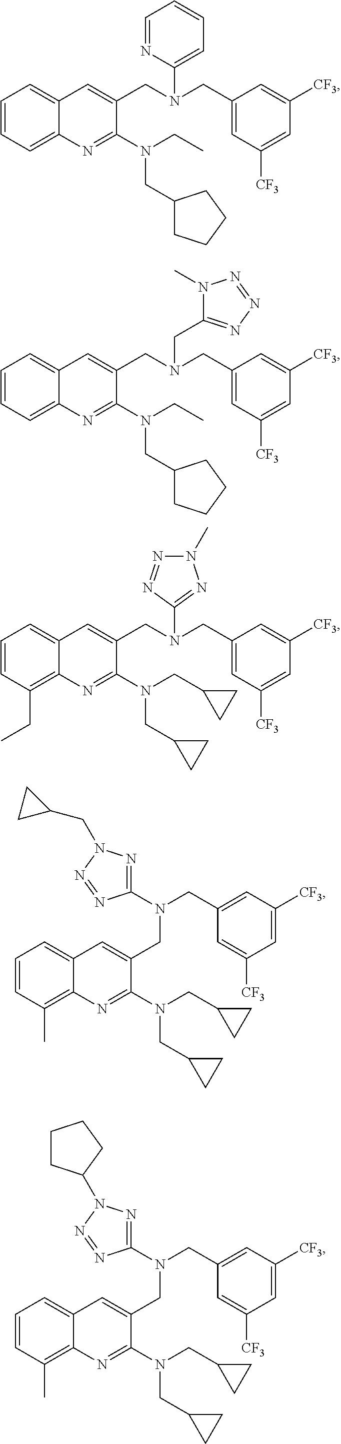 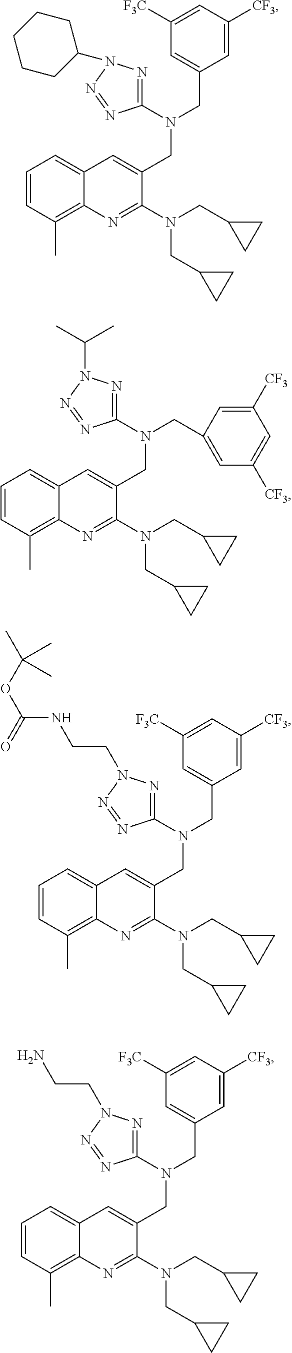 [SMILES: Cc1cccc2cc(CN(Cc3cc(C(F)(F)F)cc(C(F)(F)F)c3)c3nnn(C4CCCCC4)n3)c(N(CC3CC3)CC3CC3)nc12 ; Cc1cccc2cc(CN(Cc3cc(C(F)(F)F)cc(C(F)(F)F)c3)c3nnn(C(C)C)n3)c(N(CC3CC3)CC3CC3)nc12 ; Cc1cccc2cc(CN(Cc3cc(C(F)(F)F)cc(C(F)(F)F)c3)c3nnn(CCNC(=O)OC(C)(C)C)n3)c(N(CC3CC3)CC3CC3)nc12 ; Cc1cccc2cc(CN(Cc3cc(C(F)(F)F)cc(C(F)(F)F)c3)c3nnn(CCN)n3)c(N(CC3CC3)CC3CC3)nc12] 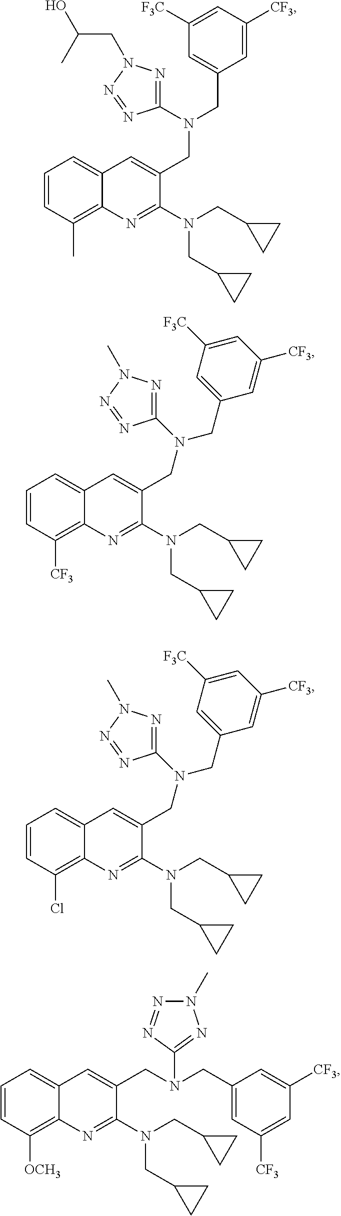 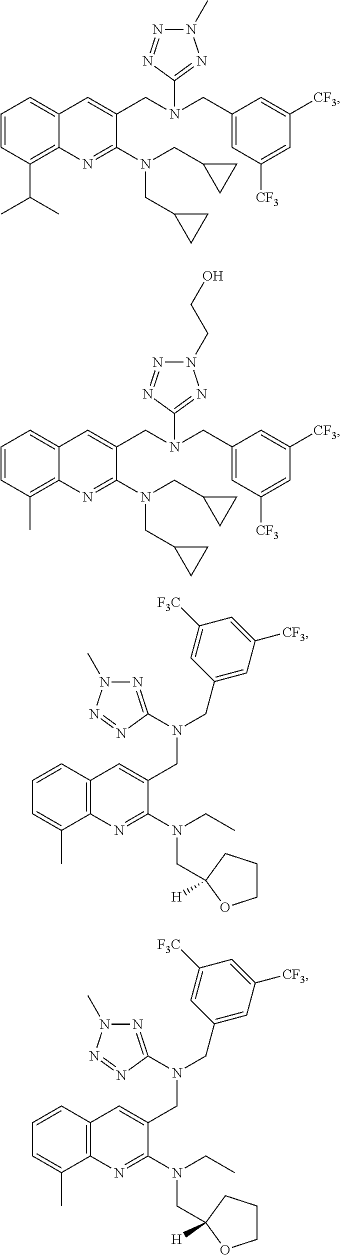 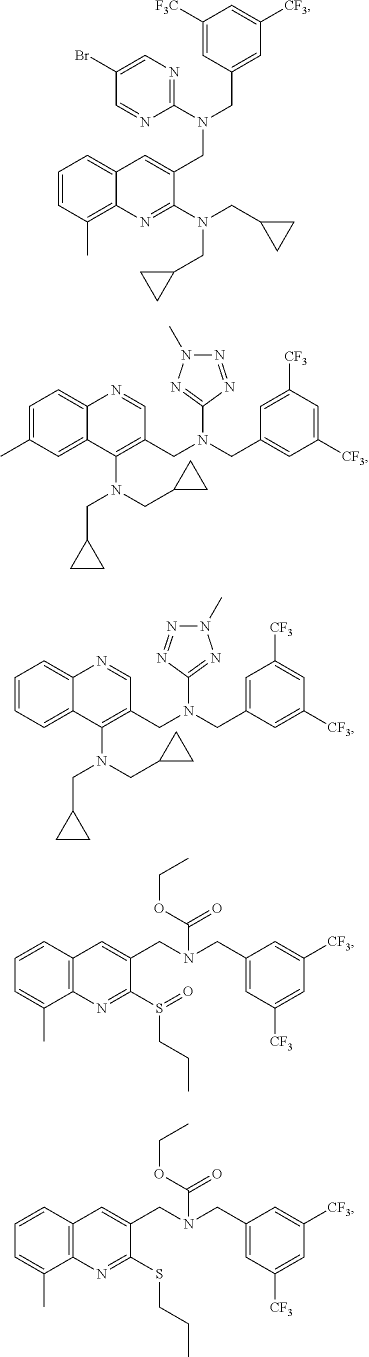 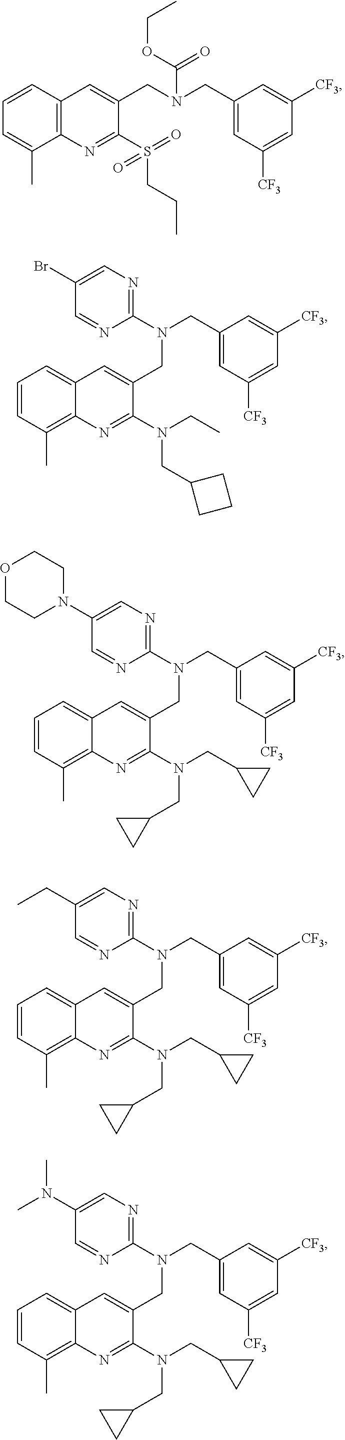 [SMILES: CCCS(=O)(=O)c1nc2c(C)cccc2cc1CN(Cc1cc(C(F)(F)F)cc(C(F)(F)F)c1)C(=O)OCC ; CCN(CC1CCC1)c1nc2c(C)cccc2cc1CN(Cc1cc(C(F)(F)F)cc(C(F)(F)F)c1)c1ncc(Br)cn1 ; Cc1cccc2cc(CN(Cc3cc(C(F)(F)F)cc(C(F)(F)F)c3)c3ncc(N4CCOCC4)cn3)c(N(CC3CC3)CC3CC3)nc12 ; CCc1cnc(N(Cc2cc(C(F)(F)F)cc(C(F)(F)F)c2)Cc2cc3cccc(C)c3nc2N(CC2CC2)CC2CC2)nc1 ; Cc1cccc2cc(CN(Cc3cc(C(F)(F)F)cc(C(F)(F)F)c3)c3ncc(N(C)C)cn3)c(N(CC3CC3)CC3CC3)nc12] 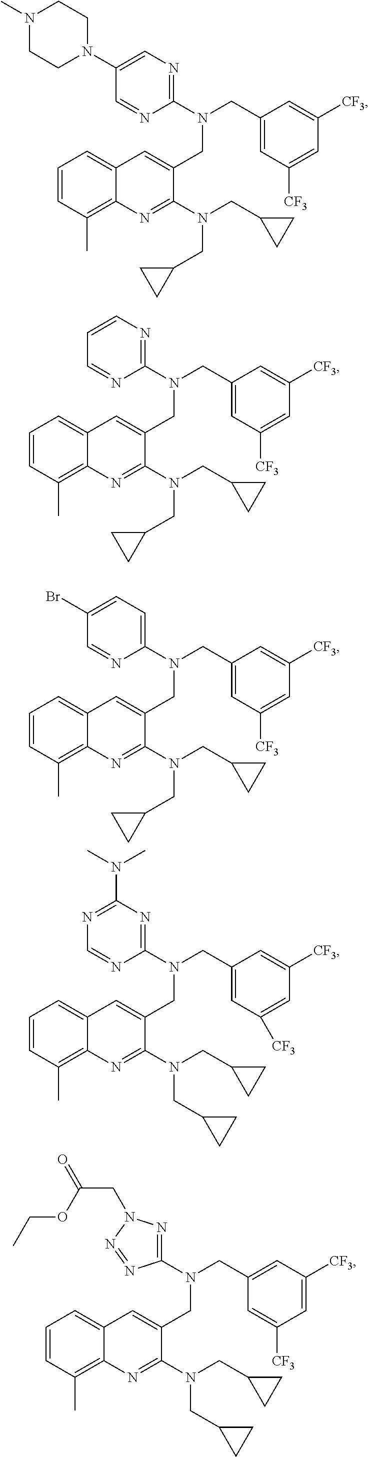 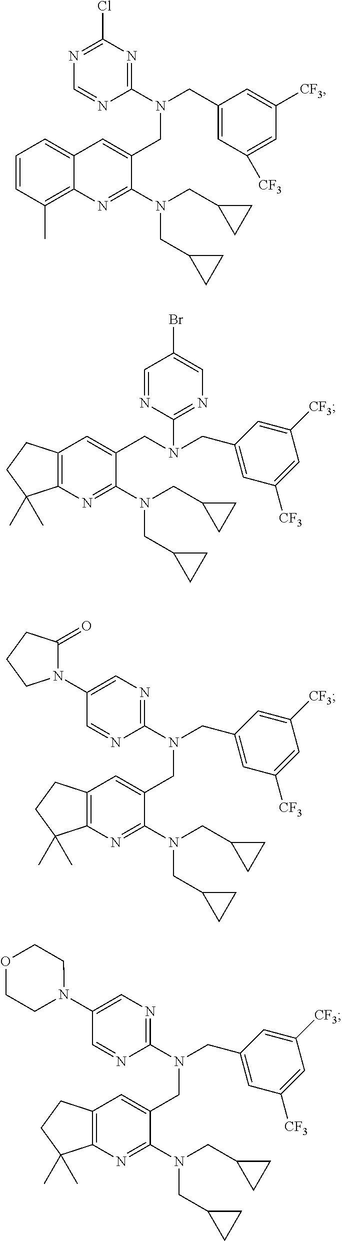 [SMILES: Cc1cccc2cc(CN(Cc3cc(C(F)(F)F)cc(C(F)(F)F)c3)c3ncnc(Cl)n3)c(N(CC3CC3)CC3CC3)nc12 ; CC1(C)CCc2cc(CN(Cc3cc(C(F)(F)F)cc(C(F)(F)F)c3)c3ncc(Br)cn3)c(N(CC3CC3)CC3CC3)nc21 ; CC1(C)CCc2cc(CN(Cc3cc(C(F)(F)F)cc(C(F)(F)F)c3)c3ncc(N4CCCC4=O)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21] 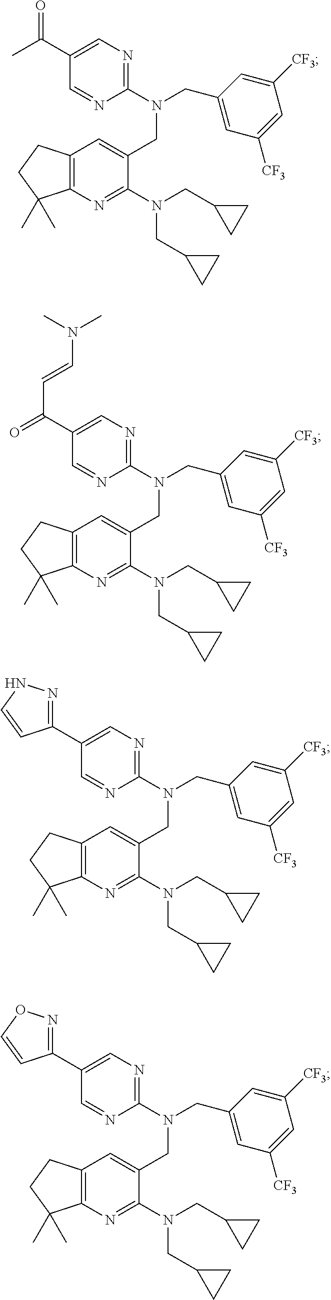 [SMILES: CC(=O)c1cnc(N(Cc2cc(C(F)(F)F)cc(C(F)(F)F)c2)Cc2cc3c(nc2N(CC2CC2)CC2CC2)C(C)(C)CC3)nc1 ; CN(C)/C=C/C(=O)c1cnc(N(Cc2cc(C(F)(F)F)cc(C(F)(F)F)c2)Cc2cc3c(nc2N(CC2CC2)CC2CC2)C(C)(C)CC3)nc1 ; CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(-c4ccon4)cn3)c(N(CC3CC3)CC3CC3)nc21] 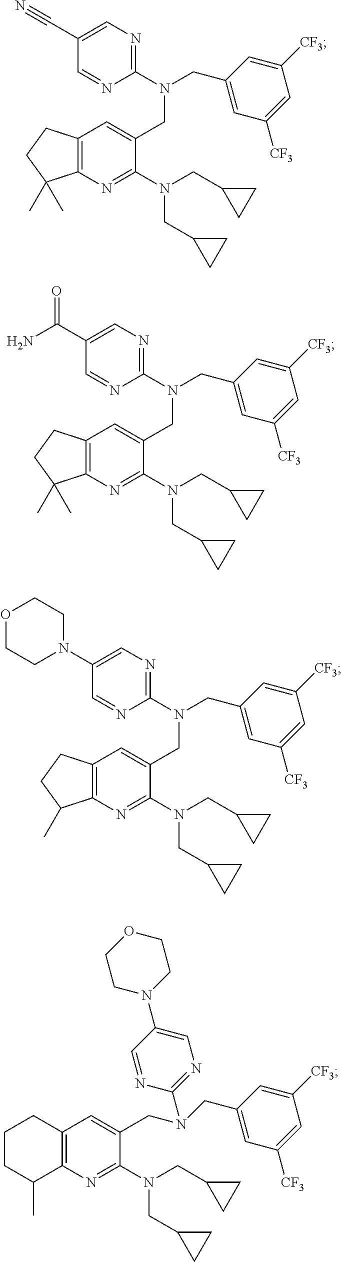 [SMILES: CC1(C)CCc2cc(CN(Cc3cc(C(F)(F)F)cc(C(F)(F)F)c3)c3ncc(C#N)cn3)c(N(CC3CC3)CC3CC3)nc21 ; CC1(C)CCc2cc(CN(Cc3cc(C(F)(F)F)cc(C(F)(F)F)c3)c3ncc(C(N)=O)cn3)c(N(CC3CC3)CC3CC3)nc21 ; CC1CCc2cc(CN(Cc3cc(C(F)(F)F)cc(C(F)(F)F)c3)c3ncc(N4CCOCC4)cn3)c(N(CC3CC3)CC3CC3)nc21 ; CC1CCCc2cc(CN(Cc3cc(C(F)(F)F)cc(C(F)(F)F)c3)c3ncc(N4CCOCC4)cn3)c(N(CC3CC3)CC3CC3)nc21] 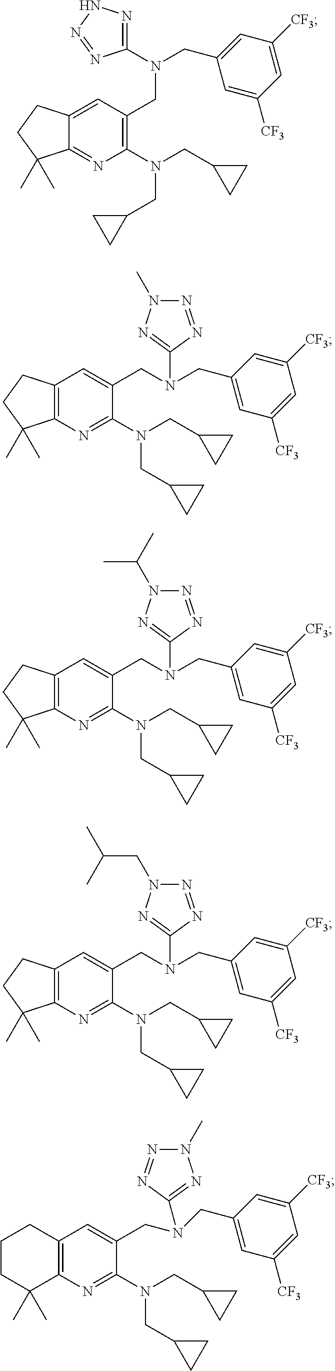 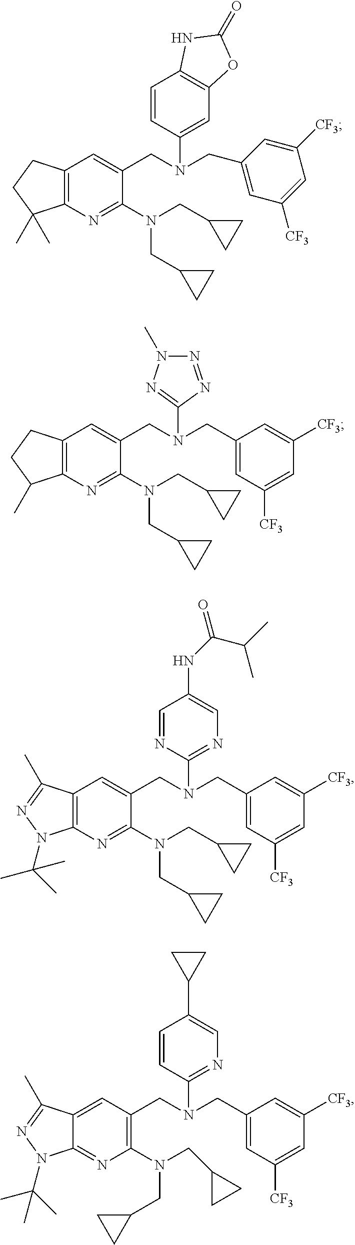 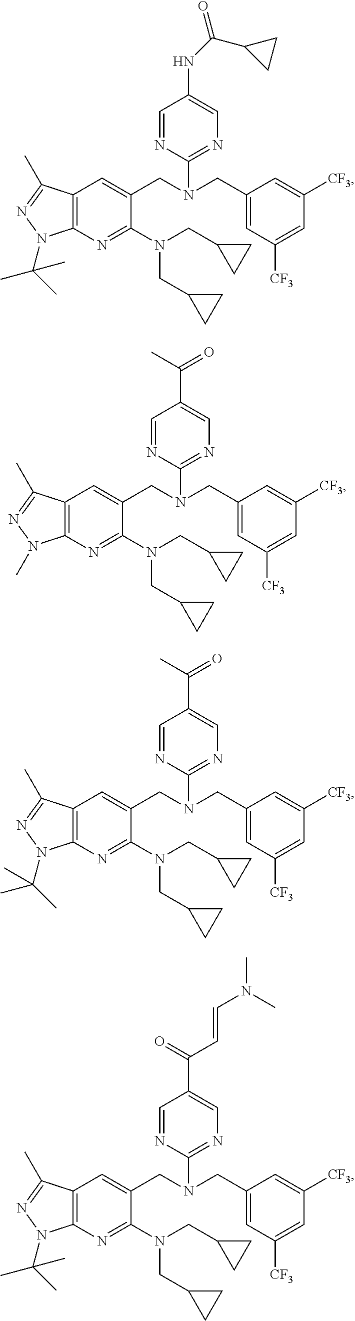 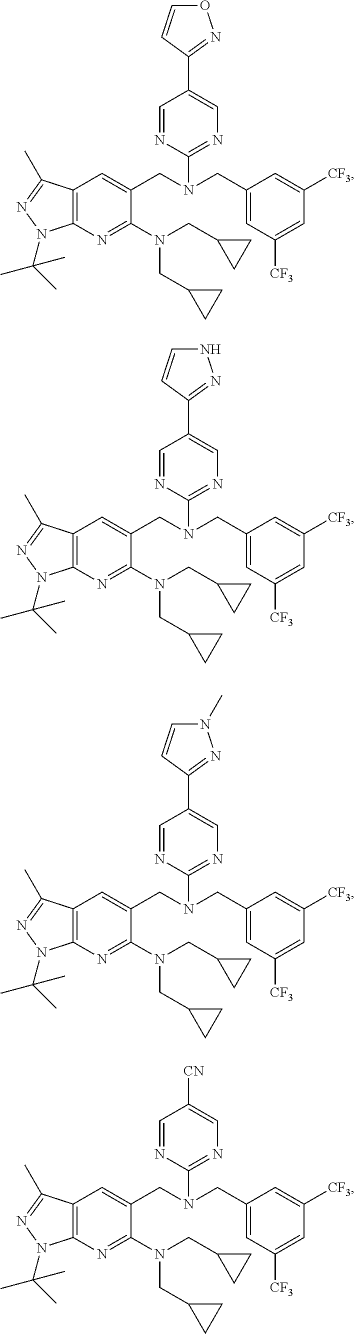 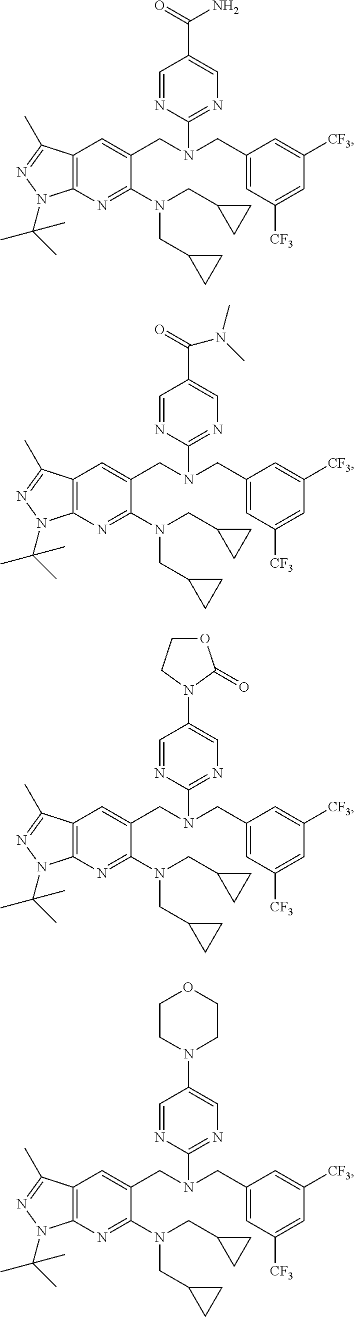 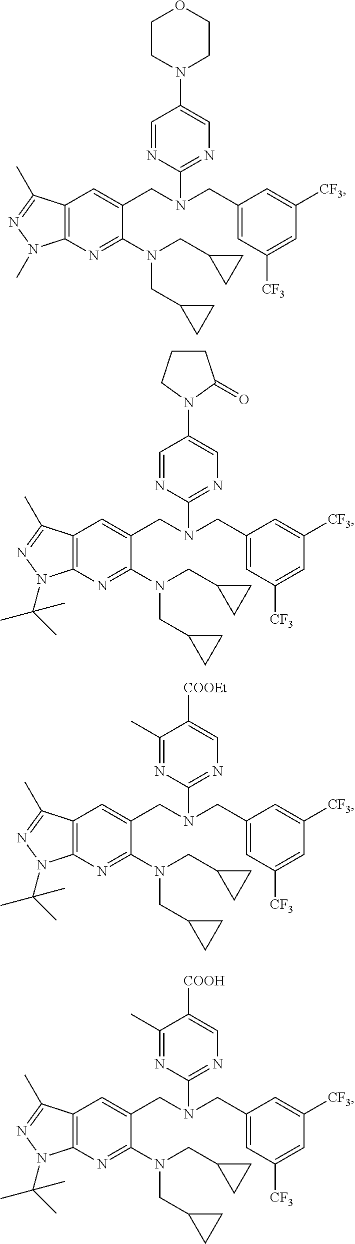 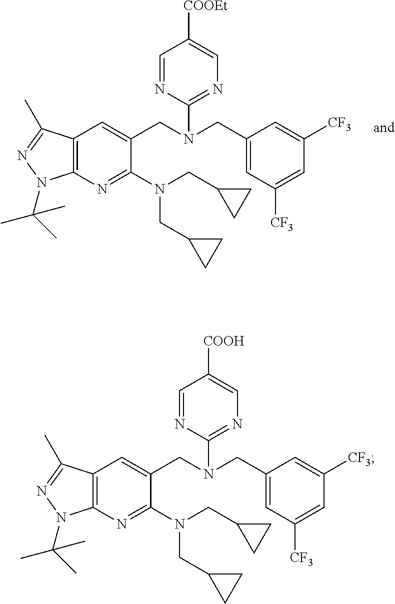 [SMILES: CCOC(=O)c1cnc(N(Cc2cc(C(F)(F)F)cc(C(F)(F)F)c2)Cc2cc3c(C)nn(C(C)(C)C)c3nc2N(CC2CC2)CC2CC2)nc1 ; Cc1nn(C(C)(C)C)c2nc(N(CC3CC3)CC3CC3)c(CN(Cc3cc(C(F)(F)F)cc(C(F)(F)F)c3)c3ncc(C(=O)O)cn3)cc12]
or a stereoisomer thereof or a pharmaceutically acceptable salt thereof.

28. The composition according to claim 1, wherein the CETP inhibitor is selected from a group consisting of: 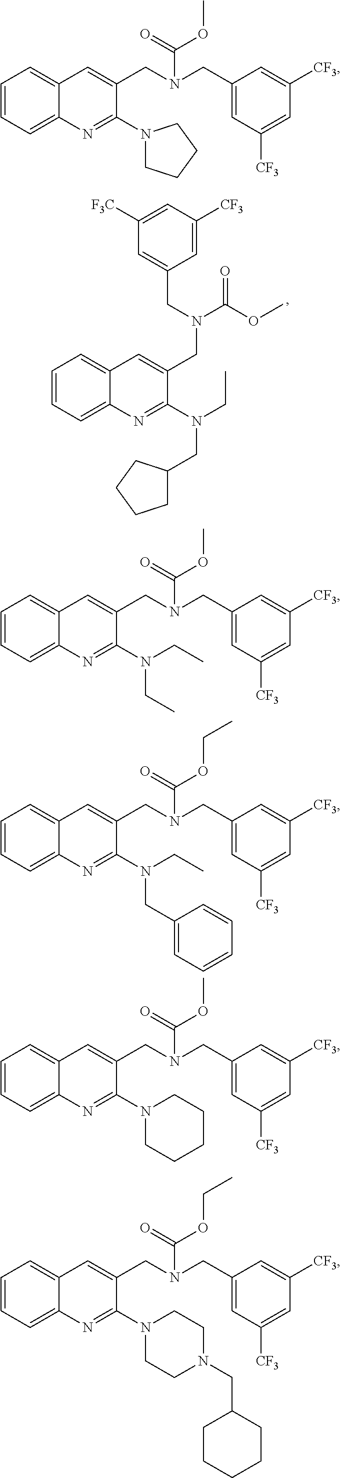 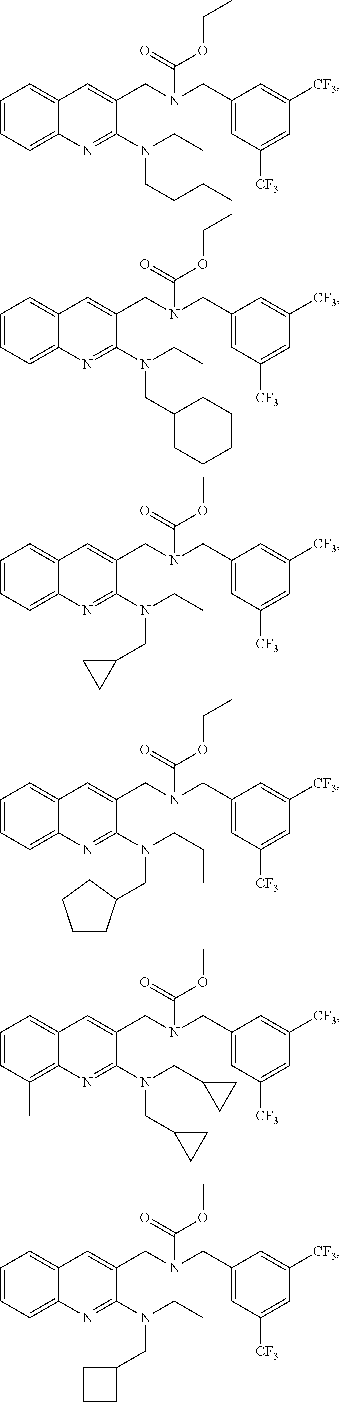 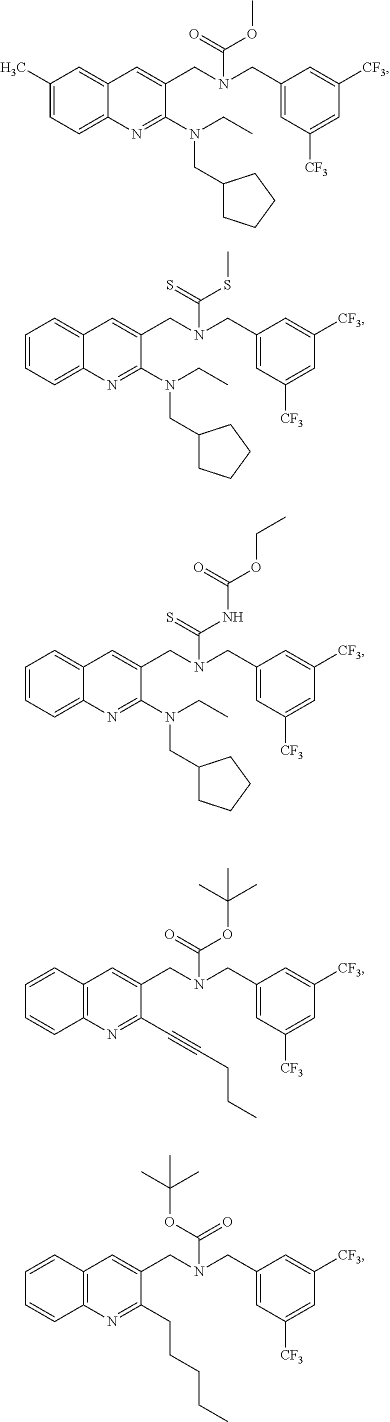 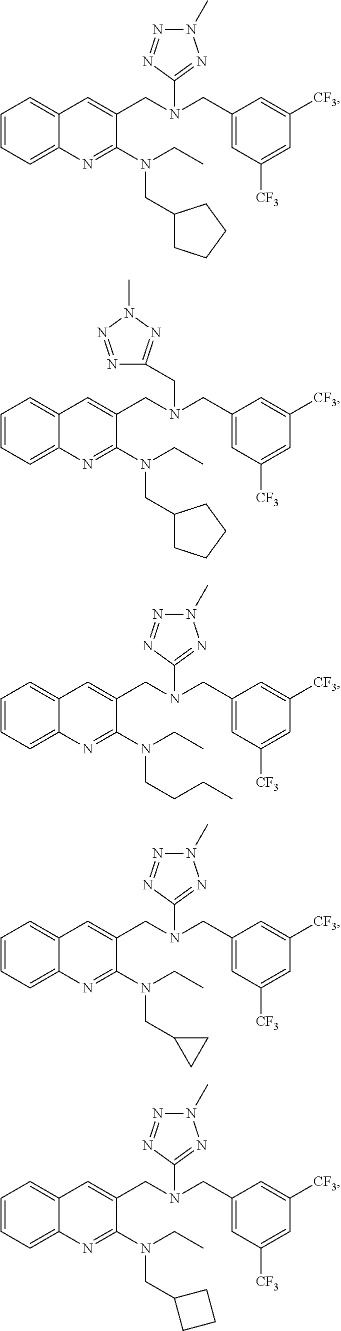 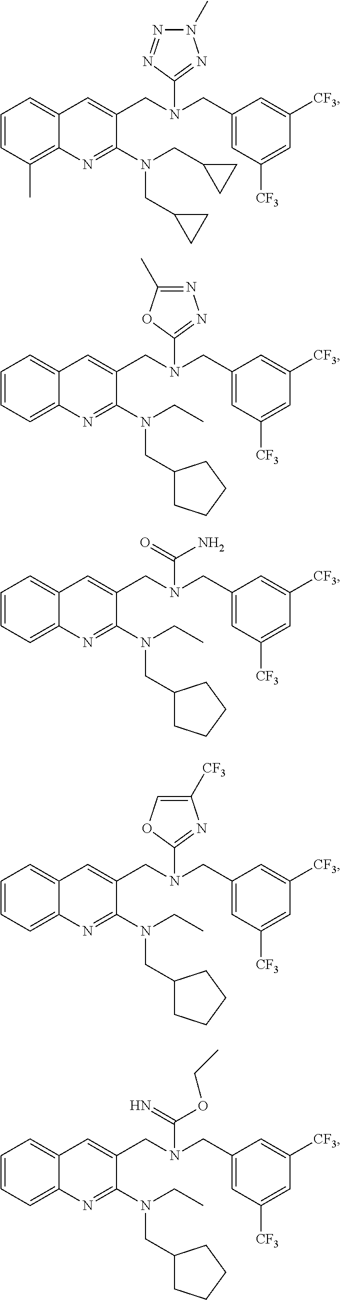 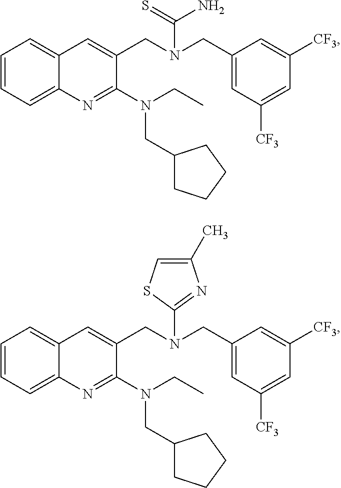 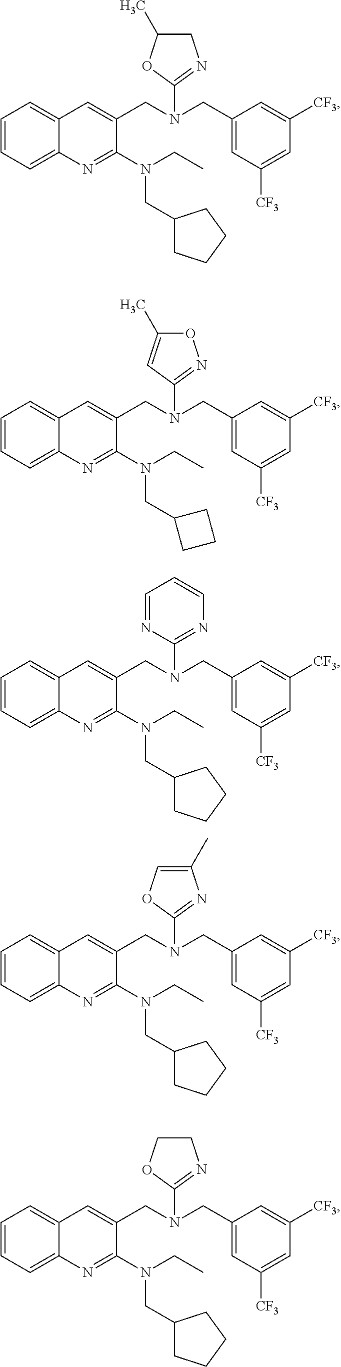 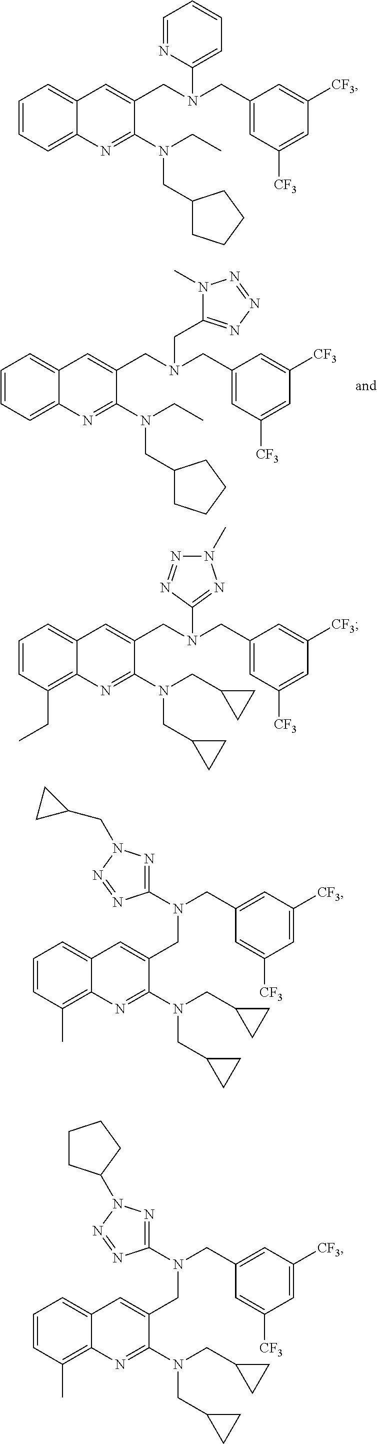 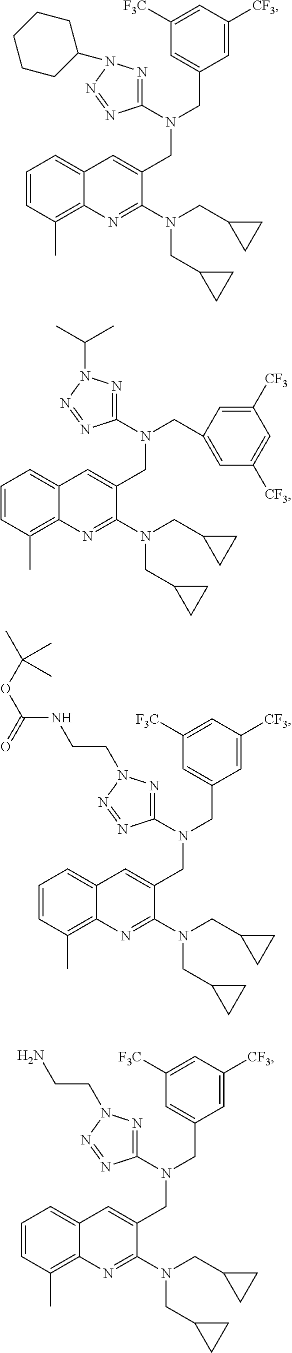 [SMILES: Cc1cccc2cc(CN(Cc3cc(C(F)(F)F)cc(C(F)(F)F)c3)c3nnn(C4CCCCC4)n3)c(N(CC3CC3)CC3CC3)nc12 ; Cc1cccc2cc(CN(Cc3cc(C(F)(F)F)cc(C(F)(F)F)c3)c3nnn(C(C)C)n3)c(N(CC3CC3)CC3CC3)nc12 ; Cc1cccc2cc(CN(Cc3cc(C(F)(F)F)cc(C(F)(F)F)c3)c3nnn(CCNC(=O)OC(C)(C)C)n3)c(N(CC3CC3)CC3CC3)nc12 ; Cc1cccc2cc(CN(Cc3cc(C(F)(F)F)cc(C(F)(F)F)c3)c3nnn(CCN)n3)c(N(CC3CC3)CC3CC3)nc12] 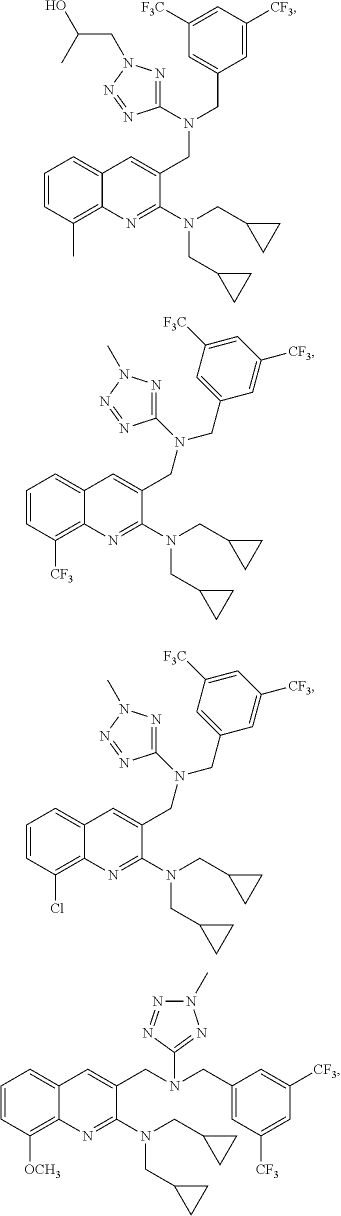 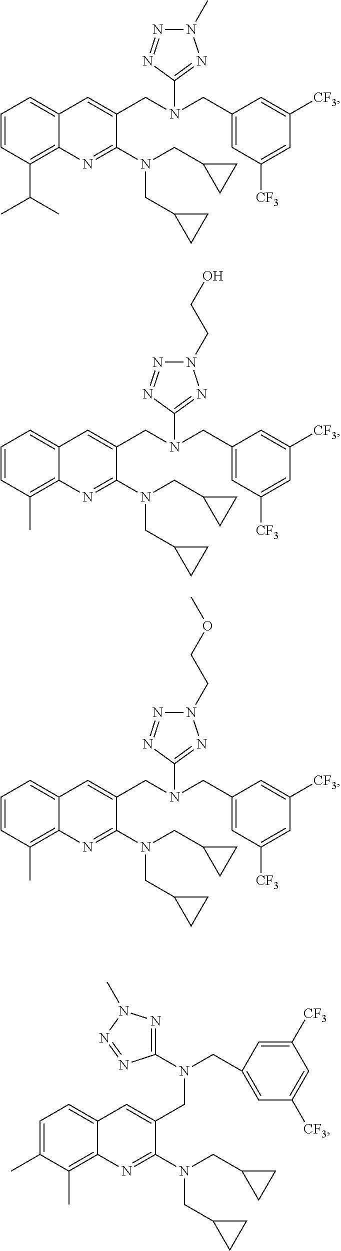 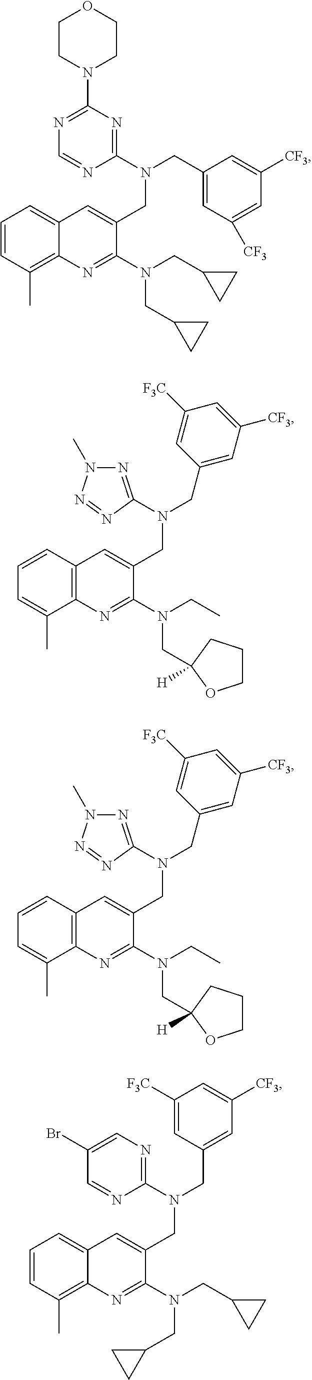 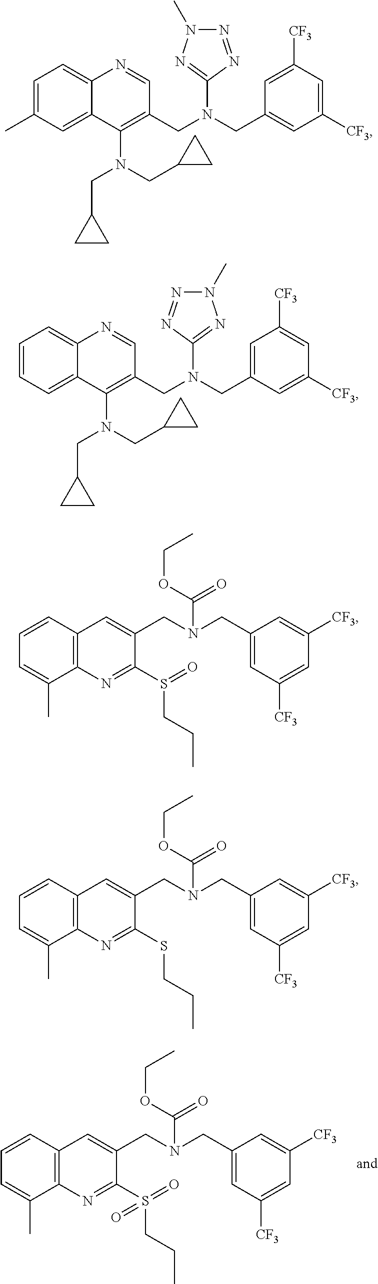 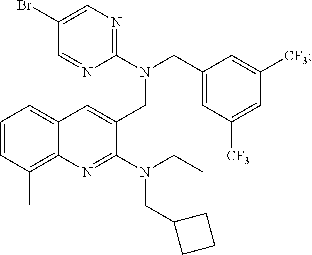 [SMILES: CCN(CC1CCC1)c1nc2c(C)cccc2cc1CN(Cc1cc(C(F)(F)F)cc(C(F)(F)F)c1)c1ncc(Br)cn1]
or a stereoisomer thereof or a pharmaceutically acceptable salt thereof.

29. The composition according to claim 1, wherein the CETP inhibitor is selected from a group consisting of: 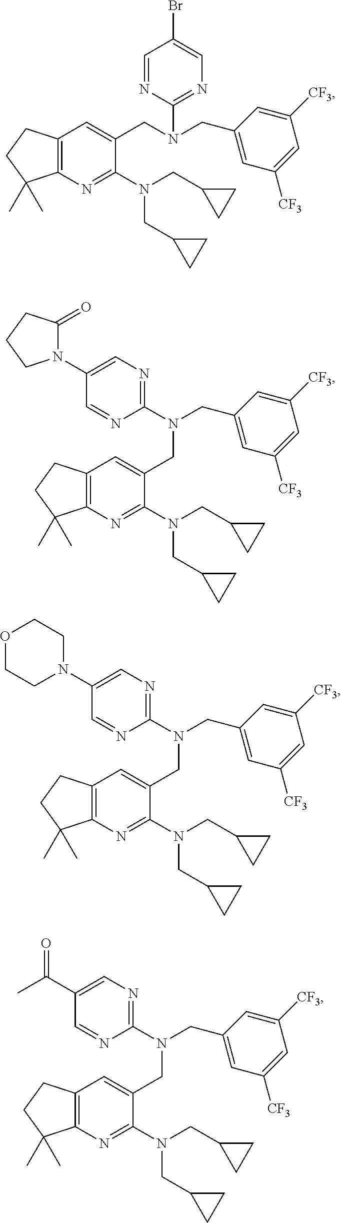 [SMILES: CC1(C)CCc2cc(CN(Cc3cc(C(F)(F)F)cc(C(F)(F)F)c3)c3ncc(Br)cn3)c(N(CC3CC3)CC3CC3)nc21 ; CC1(C)CCc2cc(CN(Cc3cc(C(F)(F)F)cc(C(F)(F)F)c3)c3ncc(N4CCCC4=O)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; CC(=O)c1cnc(N(Cc2cc(C(F)(F)F)cc(C(F)(F)F)c2)Cc2cc3c(nc2N(CC2CC2)CC2CC2)C(C)(C)CC3)nc1] 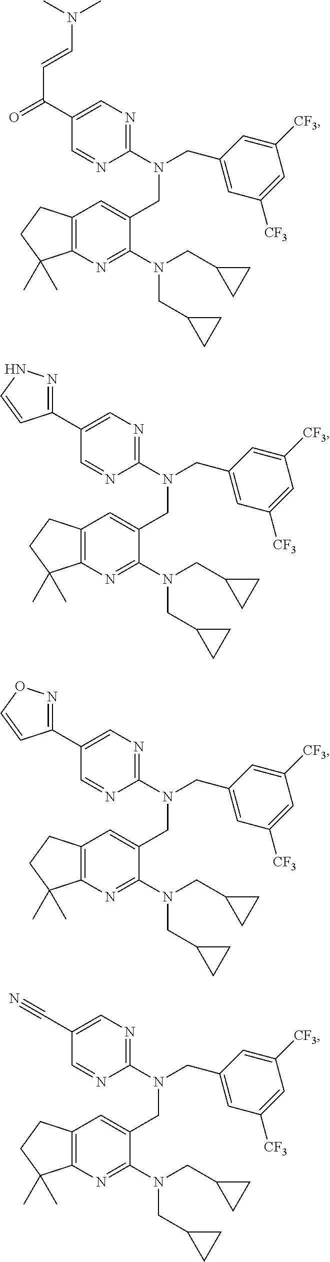 [SMILES: CN(C)/C=C/C(=O)c1cnc(N(Cc2cc(C(F)(F)F)cc(C(F)(F)F)c2)Cc2cc3c(nc2N(CC2CC2)CC2CC2)C(C)(C)CC3)nc1 ; CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(-c4ccon4)cn3)c(N(CC3CC3)CC3CC3)nc21 ; CC1(C)CCc2cc(CN(Cc3cc(C(F)(F)F)cc(C(F)(F)F)c3)c3ncc(C#N)cn3)c(N(CC3CC3)CC3CC3)nc21] 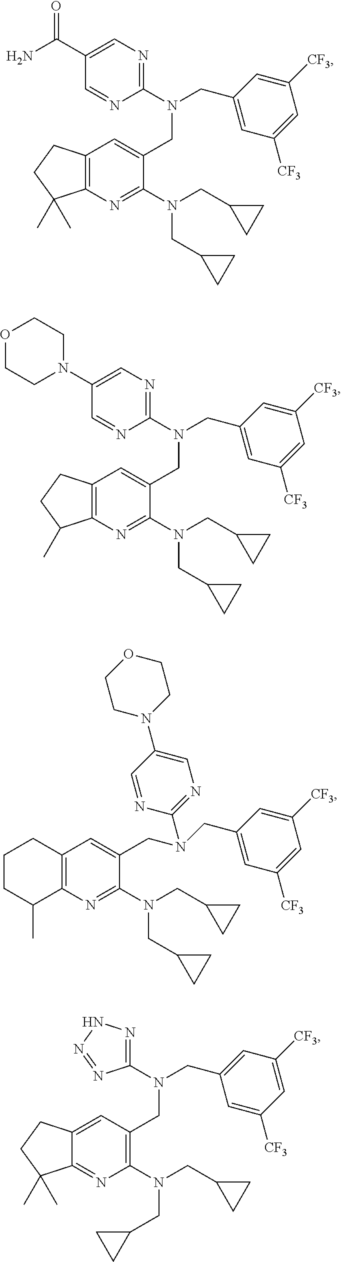 [SMILES: CC1(C)CCc2cc(CN(Cc3cc(C(F)(F)F)cc(C(F)(F)F)c3)c3ncc(C(N)=O)cn3)c(N(CC3CC3)CC3CC3)nc21 ; CC1CCc2cc(CN(Cc3cc(C(F)(F)F)cc(C(F)(F)F)c3)c3ncc(N4CCOCC4)cn3)c(N(CC3CC3)CC3CC3)nc21 ; CC1CCCc2cc(CN(Cc3cc(C(F)(F)F)cc(C(F)(F)F)c3)c3ncc(N4CCOCC4)cn3)c(N(CC3CC3)CC3CC3)nc21 ; CC1(C)CCc2cc(CN(Cc3cc(C(F)(F)F)cc(C(F)(F)F)c3)c3nn[nH]n3)c(N(CC3CC3)CC3CC3)nc21] 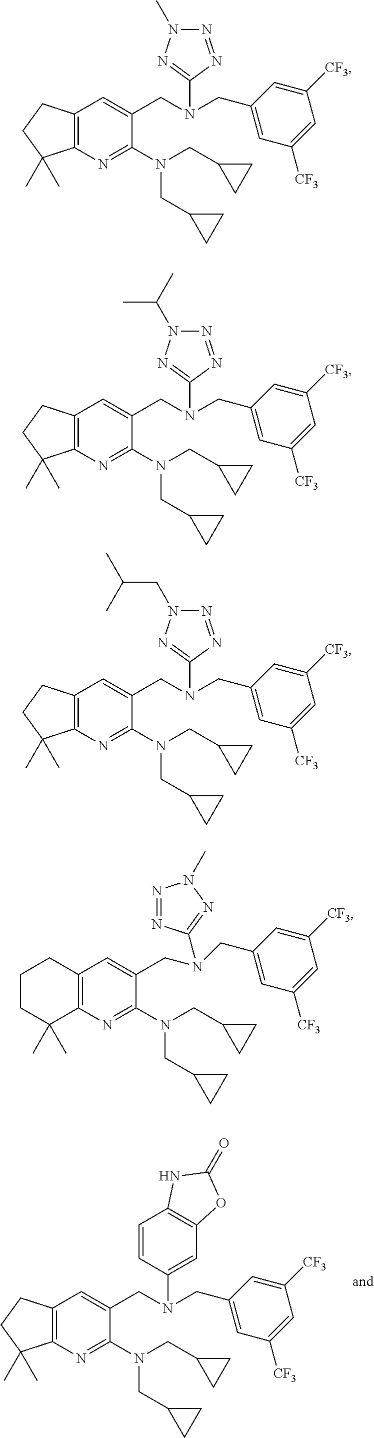 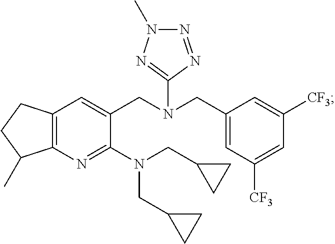 [SMILES: CC1CCc2cc(CN(Cc3cc(C(F)(F)F)cc(C(F)(F)F)c3)c3nnn(C)n3)c(N(CC3CC3)CC3CC3)nc21]
or a stereoisomer thereof or a pharmaceutically acceptable salt thereof.

30. The composition according to claim 1, wherein the CETP inhibitor is selected from a group consisting of: 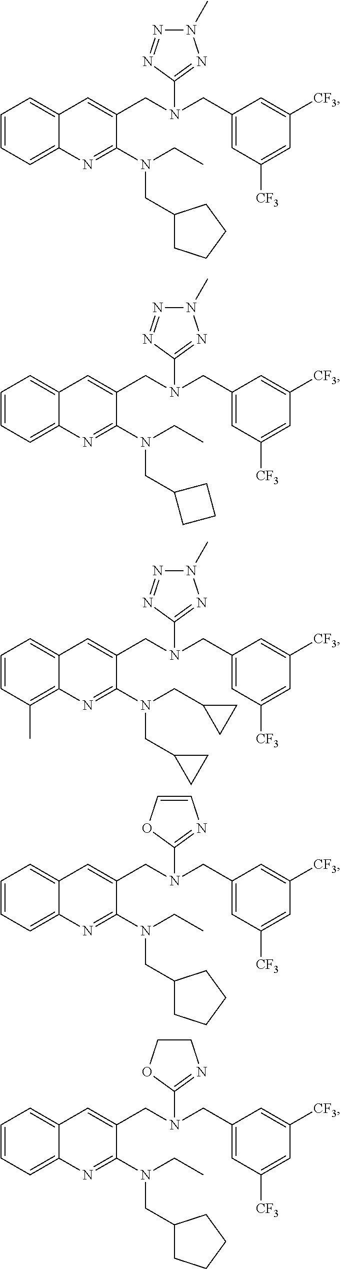 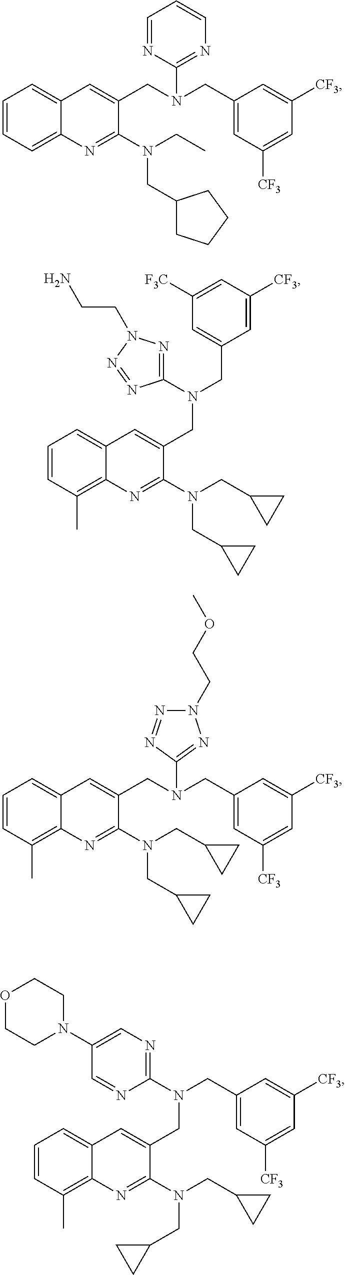 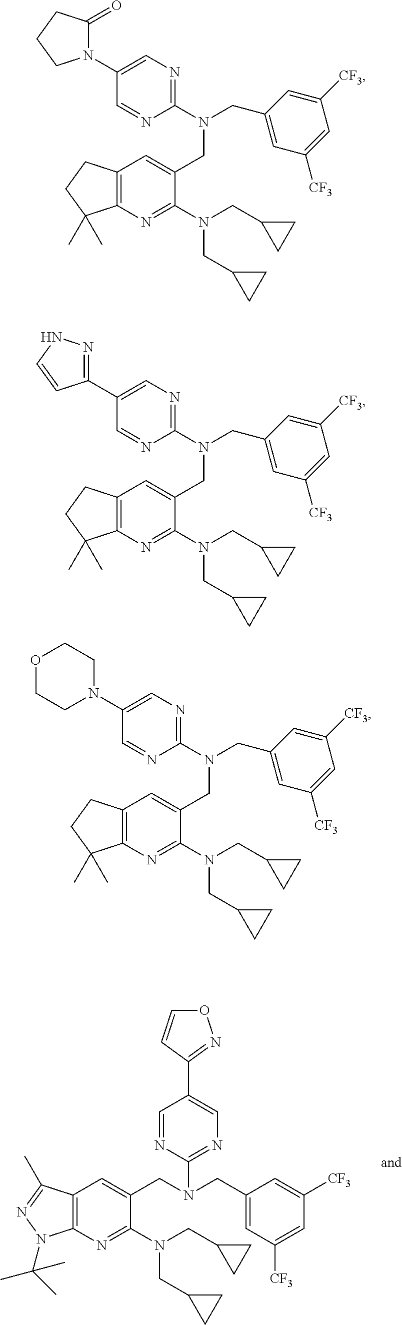 [SMILES: CC1(C)CCc2cc(CN(Cc3cc(C(F)(F)F)cc(C(F)(F)F)c3)c3ncc(N4CCCC4=O)cn3)c(N(CC3CC3)CC3CC3)nc21 ; CC1(C)CCc2cc(CN(Cc3cc(C(F)(F)F)cc(C(F)(F)F)c3)c3ncc(-c4cc[nH]n4)cn3)c(N(CC3CC3)CC3CC3)nc21 ; CC1(C)CCc2cc(CN(Cc3cc(C(F)(F)F)cc(C(F)(F)F)c3)c3ncc(N4CCOCC4)cn3)c(N(CC3CC3)CC3CC3)nc21 ; Cc1nn(C(C)(C)C)c2nc(N(CC3CC3)CC3CC3)c(CN(Cc3cc(C(F)(F)F)cc(C(F)(F)F)c3)c3ncc(-c4ccon4)cn3)cc12] 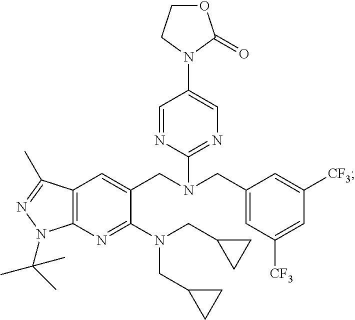 [SMILES: Cc1nn(C(C)(C)C)c2nc(N(CC3CC3)CC3CC3)c(CN(Cc3cc(C(F)(F)F)cc(C(F)(F)F)c3)c3ncc(N4CCOC4=O)cn3)cc12]
or a stereoisomer thereof or a pharmaceutically acceptable salt thereof.

31. The composition according to claim 9, wherein said composition releases not more than 50% at a period of 30 minutes in 900 ml of simplified simulated intestinal fluid having a pH of 6.5, when tested in a USP Type 2 apparatus at 25 rpm and 37° C.
